(19) United States
(12) Patent Application Publication
(10) Pub. No.: US 2016/0369340 A1
(43) Pub. Date: Dec. 22, 2016

(54) ASSAY TO MEASURE THE LEVELS OF CIRCULATING DEMETHYLATED DNA

(71) Applicant: Winthrop-University Hospital, Mineola, NY (US)

(72) Inventor: Eitan Moshe Akirav, Plainview, NY (US)

(21) Appl. No.: 14/966,526

(22) Filed: Dec. 11, 2015

Related U.S. Application Data
(60) Provisional application No. 62/090,611, filed on Dec. 11, 2014.
(60) Provisional application No. 62/090,618, filed on Dec. 11, 2014.
(60) Provisional application No. 62/109,340, filed on Jan. 29, 2015.

(51) Int. Cl. C12Q 1/68

(52) U.S. Cl. CPC C12Q 1/6883; C12Q 2600/154

(57) ABSTRACT

A method for measuring blood levels of DNA that is released upon death from specialized cells in the body, by using PCR or a quantitative probe technology to detect amplified methylated and demethylated forms of cell-specific gene DNA, representing normal tissue and cell specific origin, respectively. Using probes permits the sensitive and specific identification of demethylated cell-specific DNA patterns that are present only in the dying cells. The method offers a bioassay for detecting β cell loss in diabetes based on circulating demethylated insulin gene DNA, and circulating demethylated myelin oligodendrocyte protein (MOG) genes of oligodendrocytes in multiple sclerosis, for example, and may be useful for screening, monitoring of disease progression, and selection and monitoring of therapies.

### CROSS REFERENCE TO RELATED APPLICATIONS

The present application is a non-provisional of, and claims benefit of priority from U.S. Provisional Patent Application No. 62/090,611, filed Dec. 11, 2014, and from U.S. Provisional Patent Application No. 62/090,618, filed Dec. 11, 2014, and from U.S. Provisional Application No. 62/109,340, filed Jan. 2015, each of which is expressly incorporated herein by reference in its entirety.

### BACKGROUND OF THE INVENTION

Field

The present application relates to compositions and methods for assessing particular cell loss by quantitating DNA derived from that particular cell type, with methylation status-specific oligonucleotide probes that target Polymerase Chain Reaction (PCR)-amplified DNA sequences, or PCR primers themselves, of genes that have unique gene methylation patterns expressed by those cells.

Description of the Art

There are a number of diseases which are characterized by selective cell loss of particular cells. For example, type I diabetes can result from an autoimmune process which targets pancreatic c β cells, resulting in loss of this cell type and the insulin they produce. During cell loss, DNA from these cells is released, and some finds its way into the circulating body fluids. Because pancreatic β cells are the only significant cell type which produces significant amounts of insulin, only these cells have demethylated insulin gene DNA. Similarly in multiple sclerosis, an autoimmune process can lead to loss of oligodendrocytes, which selectively produce myelin oligodendrocyte glycoprotein. The death of oligodendrocytes is therefore associated with an increase in the level of demethylated myelin oligodendrocyte glycoprotein DNA. Other autoimmune diseases include (see, www.womenshealth.gov/publications/our-publications/fact-sheet/autoimmune-diseases.html#b):

Addison's disease: adrenal hormone insufficiency;

Alopecia areata: The immune system attacks hair follicles (the structures from which hair grows). It usually does not threaten health, but it can greatly affect the way a person looks.

Antiphospholipid antibody syndrome (aPL): A disease that causes problems in the inner lining of blood vessels resulting in blood clots in arteries or veins.

Autoimmune hepatitis: The immune system attacks and destroys the liver cells. This can lead to scarring and hardening of the liver, and possibly liver failure.

Celiac disease: A disease in which people can't tolerate gluten, a substance found in wheat, rye, and barley, and also some medicines. When people with celiac disease eat foods or use products that have gluten, the immune system responds by damaging the lining of the small intestines.

Diabetes type 1: A disease in which your immune system attacks the cells that make insulin, a hormone needed to control blood sugar levels. As a result, your body cannot make insulin. Without insulin, too much sugar stays in your blood. Too high blood sugar can hurt the eyes, kidneys, nerves, and gums and teeth. But the most serious problem caused by diabetes is heart disease.

Graves' disease (overactive thyroid): A disease that causes the thyroid to make too much thyroid hormone.

Guillain-Barre syndrome: The immune system attacks the nerves that connect your brain and spinal cord with the rest of your body. Damage to the nerves makes it hard for them to transmit signals. As a result, the muscles have trouble responding to the brain.

Hashimoto's disease (underactive thyroid): A disease that causes the thyroid to not make enough thyroid hormone.

Hemolytic anemia: The immune system destroys the red blood cells. Yet the body can't make new red blood cells fast enough to meet the body's needs. As a result, your body does not get the oxygen it needs to function well, and your heart must work harder to move oxygen-rich blood throughout the body.

Idiopathic thrombocytopenic purpura (ITP): A disease in which the immune system destroys blood platelets, which are needed for blood to clot.

Inflammatory bowel disease (IBD): A disease that causes chronic inflammation of the digestive tract. Crohn's disease and ulcerative colitis are the most common forms of IBD.

Inflammatory myopathies: A group of diseases that involve muscle inflammation and muscle weakness. Polymyositis and dermatomyositis are 2 types more common in women than men.

Multiple sclerosis (MS): A disease in which the immune system attacks the protective coating around the nerves. The damage affects the brain and spinal cord.

Myasthenia gravis (MG): A disease in which the immune system attacks the nerves and muscles throughout the body.

Primary biliary cirrhosis: The immune system slowly destroys the liver's bile ducts. Bile is a substance made in the liver. It travels through the bile ducts to help with digestion. When the ducts are destroyed, the bile builds up in the liver and hurts it. The damage causes the liver to harden and scar, and eventually stop working.

Psoriasis: A disease that causes new skin cells that grow deep in your skin to rise too fast and pile up on the skin surface.

Reactive Arthritis: Inflammation of joints, urethra, and eyes; may cause sores on the skin and mucus membranes

Rheumatoid arthritis: A disease in which the immune system attacks the lining of the joints throughout the body.

Scleroderma: A disease causing abnormal growth of connective tissue in the skin and blood vessels.

Sjögren's syndrome: A disease in which the immune system targets the glands that make moisture, such as tears and saliva.

Systemic lupus erythematosus: A disease that can damage the joints, skin, kidneys, heart, lungs, and other parts of the body. Also called SLE or lupus.

Vitiligo: The immune system destroys the cells that give your skin its color. It also can affect the tissue inside your mouth and nose.

Epigenetic modifications of DNA are used by various cell types to control tissue-specific gene expression. These modifications include histone acetylation/deacetylation and DNA methylation (Klose et al., 2006, Trends Biochem. Sci. 31 :89-97; Bartke et al., 2010, Cell 143:470-484; Wang et al., 2007, Trends Mol. Med. 13 :373-380). Methylation of DNA sequences occurs in CpG dinucleotide sites to maintain a transcriptionally repressive chromatin configuration, whereas demethylation results in a transcriptionally permissive configuration (Miranda et al., 2007, J. Cell Physiol. 213:384-390). Beta cells express insulin, and thus, maintain a transcriptionally-permissive hypomethylated regulatory region for the insulin gene (INS). Indeed, Genomic DNA sequences near the insulin gene are methylated in non-βcell, cell types. Ley, Timothy J., et al. “DNA methylation and regulation of the human beta-globin-like genes in mouse erythroleukemia cells containing human chromosome 11.” Proceedings of the National Academy of Sciences 81.21 (1984): 6618-6622.) Therefore, the presence of hypomethylated insulin gene DNA outside of the pancreas of a subject correlated with the release of hypomethylated insulin gene DNA from dead and dying (e.g., apoptotic) β cells. Id. and Kuroda A, Rauch T A, Todorov I, Ku H T, Al-Abdullah I H, et al. (2009) Insulin Gene Expression Is Regulated by DNA Methylation. PLoS ONE 4(9): e6953. doi:10.1371/journal.pone.0006953.

Differential methylation of oncogenes has been used to identify microsatellite instability in patients with colon cancer, detection of differentially methylated DNA in the serum of cancer patients has been used as a biomarker for cancer diagnosis, and beta cell pathology in type I diabetes (Grady et al., 2001, Cancer Res. 61:900-902; Wallner et al., 2006, Clin Cancer Res. 12:7347-7352; Muller et al., 2003, Cancer Res. 63:7641 -7645; Akirav, E. M., J. Lebastchi, E. M. Galvan, O. Henegariu, M. Akirav, V. Ablamunits, P. M. Lizardi, and K. C. Herold. 2011. Detection of beta cell death in diabetes using differentially methylated circulating DNA. Proc Natl Acad Sci U S A 108:19018-19023). Previous studies have relied on the detection of serum-derived tissue-specific epigenetic modifications to identify DNA released from those cells when they die.

The loss of insulin producing β cells results in glucose intolerance and the development of Type 1(T1D) and Type 2 (T2D) diabetes. Eitan Akirav, Jake A. Kushner and Kevan C. Herold, β-Cell Mass and Type 1 Diabetes Going, Going, Gone?“, doi: 10.2337/db07-1817 Diabetes November 2008 vol. 57 no. 11 2883-2888. Currently, evaluation of β cell mass is carried out by measuring β cell products such as c-peptide. While useful, these measures do not provide real time information about active β cell loss. Beta cells express insulin, and thus, maintain a transcriptionally-permissive hypomethylated regulatory region for the insulin gene (INS). Indeed, Genomic DNA sequences near the insulin gene are methylated in non-βcell types. Ley, Timothy J., et al. “DNA and regulation of the human beta-globin-like genes in mouse erythroleukemia cells containing human chromosome 11.” Proceedings of the National Academy of Sciences 81.21 (1984): 6618-6622.). Therefore, the presence of hypomethylated insulin gene DNA outside of the pancreas of a subject correlated with the release of hypomethylated insulin gene sequencing of a cytogenetically normal acute myeloid leukemia genome.” Nature 456.7218 (2008): 66-72.

Multiple sclerosis (MS) is an autoimmune disease of the central nervous system (CNS) characterized by impaired physical and mental functions. MS can be divided into different disease subtypes all of which display injury of the grey and white matter of the brain, as well as, the spinal cord (1-3). Current biomarkers of MS include magnetic resonance imagining and immunological markers, which are used together with clinical symptoms to diagnose the disease. Despite these advancements, recent studies report a relatively high rate of MS misdiagnosis, which may lead to inadequate care.

The McDonald Criteria is used to diagnose MS (4). Recent advancements in magnetic resonance imaging (MRI) have increased our ability to identify brain lesions (5, 6). Despite these advancements, recent studies report a relatively high rate of MS misdiagnosis (7-9), which may lead to inadequate care. The use of brain lesions as a biomarker of disease progression is limited by the fact that these lesions may form well after disease onset; thereby, limiting early disease diagnosis and clinical intervention.

Several cell types affected by MS include neurons, microglia, and oligodendrocytes (ODCs) (3, 10). ODCs, which form the myelin sheath, are targeted directly by immune cells that lead to cell loss (1, 11). This loss is associated with decrease myelination and impaired nerve cell conductivity and function, while remyelination is often associated with ODC recovery. ODC loss is observed in nearly all MS disease subtypes (3) ability to detect ODC loss may provide a novel biomarker for MS development, progression, and clinical response to therapy. In this disclosure we described a method for measuring ODC can be detected in the blood of patients with MS and in animal models of the disease.

DNA methylation is a basic mechanism by which cells regulate gene expression, and while all cells share an identical DNA sequence, DNA methylation varies considerably according to cell function. In general, DNA hypermethylation is association with reduced gene expression, while DNA demethylation is association with increased gene expression. ODCs are myelin producing cells that serve a primary target of the immune system in the CNS (11). Myelin oligodendrocyte glycoprotein (MOG), a key component of the myelin sheath, is produced by ODCs and has long been studied as a primary antigen in MS. MOG is predominantly expressed by ODC, making it a good biomarker of ODC loss.

See, U.S. Patent, Published Patent Application and PCT Publication Nos. 6,492,144; 6,835,541; 6,929,938; 6,989,245; 7,037,652; 7,098,015; 7,192,736; 7,301,016; 7,442,506; 7,449,297; 7,459,274; 7,572,886; 7,611,870; 7,618,936; 7,696,316; 7,718,364; 7,727,720; 7,749,702; 7,763,242; 7,781,161; 7,781,413; 7,794,929; 7,829,283; 7,858,317; 7,879,989; 7,951,563; 7,981,603; 8,034,792; 8,088,581; 8,150,626; 8,150,627; 8,163,279; 8,163,896; 8,207,316; 8,211,700; 8,268,549; 8,314,068; 8,338,416; 8,361,731; 8,367,362; 8,367,806; 8,377,888; 8,404,640; 8,440,404; 8,445,211; 8,450,061; 8,455,221; 8,460,872; 8,470,307; 8,497,066; 8,513,401; 8,568,984; 8,586,313; 8,598,135; 8,614,063; 8,664,194; 8,680,069; 8,688,388; 8,691,507; 8,700,338; 8,703,425; 8,710,200; 8,715,926; 8,728,815; 8,735,078; 8,754,062; 8,756,020; 8,771,948; 8,784,772; 8,822,663; 8,841,256; 8,859,502; 8,895,245; 8,900,565; 8,900,829; 8,927,209; 8,962,236; 8,962,247; 8,969,313; 8,980,864; 8,992,910; 8,999,380; 9,050,297; 9,061,059; 9,086,403; 9,089,604; 9,090,944; 9,095,552; 9,107,886; 9,114,113; 9,127,312; 9,127,317; 9,141,756; 9,145,438; 9,149506; 9,173,961; 9,175,331; 9,181,317; 9,181,319; 9,186,372; 9,187,780; 9,192,651; RE39920; 20020006653; 20020009777; 20020037538; 20020039736; 20020115090; 20020119466; 20020119478; 20030068616; 20030069199; 20030087382; 20030092095; 20030104358; 20030138783; 20030186337; 20030224376; 20040002082; 20040006016; 20040022764; 20040023206; 20040023207; 20040033509; 20040058355; 20040081976; 20040219559; 20050003341; 20050020527; 20050026183; 20050164233; 20050164246; 20050191640; 20050202490; 20050221314; 20050255458; 20050266470; 20050266552; 20050272065; 20050288226; 20060003359; 20060009632; 20060051768; 20060088907; 20060121452; 20060160105; 20060211752; 20060257905; 20060286576; 20070037203; 20070042365; 20070042384; 20070141582; 20070178458; 20070178478; 20070196337; 20070231797; 20070238115; 20070281934; 20080108093; 20080113399; 20080138313; 20080175822; 20080213781; 20080242638; 20080254474; 20080261217; 20080299551; 20080305473; 20090005268; 20090047212; 20090047656; 20090068657; 20090075260; 20090105145; 20090136944; 20090142302; 20090155786; 20090162329; 20090162836; 20090186359; 20090186360; 20090191548; 20090215066; 20090220980; 20090232767; 20090252742; 20090263860; 20090264306; 20090298910; 20090317810; 20090326209; 20100004304; 20100021482; 20100035246; 20100062447; 20100068720; 20100129828; 20100129844; 20100130375; 20100130418; 20100135970; 20100136010; 20100136539; 20100136571; 20100136572; 20100143902; 20100143929; 20100151456; 20100151468; 20100172880; 20100184027; 20100191040; 20100227319; 20100233703; 20100261180; 20100304372; 20100311683; 20100317000; 20100330567; 20110003753; 20110003885; 20110014204; 20110028333; 20110039719; 20110053882; 20110076726; 20110091970; 20110097727; 20110104695; 20110104701; 20110117111; 20110158975; 20110159509; 20110160091; 20110160216; 20110165565; 20110165581; 20110171637; 20110190161; 20110195021; 20110207129; 20110212444; 20110229876; 20110244458; 20110294699; 20120009601; 20120053073; 20120071418; 20120094844; 20120094925; 20120100997; 20120108445; 20120142546; 20120148561; 20120178918; 20120184449; 20120189540; 20120196827; 20120202202; 20120238610; 20120264618; 20120276542; 20120322072; 20120329712; 20130011380; 20130023430; 20130040906; 20130065836; 20130084286; 20130084287; 20130084328; 20130085115; 20130085681; 20130102758; 20130103320; 20130129668; 20130130923; 20130143969; 20130150253; 20130156727; 20130156814; 20130157879; 20130164279; 20130189684; 20130230850; 20130230858; 20130237445; 20130261983; 20130288241; 20130288244; 20130288918; 20130296183; 20130303555; 20130304392; 20130309666; 20130309668; 20130310260; 20130310334; 20130315860; 20130316931; 20130324543; 20130325360; 20130338020; 20130338933; 20140031257; 20140037576; 20140039154; 20140080715; 20140093873; 20140100792; 20140128283; 20140155279; 20140178339; 20140179805; 20140180594; 20140186827; 20140187537; 20140194317; 20140206014; 20140206015; 20140222443; 20140235474; 20140242588; 20140255418; 20140271657; 20140272967; 20140272968; 20140274753; 20140274908; 20140275081; 20140302042; 20140315203; 20140322354; 20140322719; 20140328863; 20140349291; 20140363812; 20140371188; 20150004158; 20150005176; 20150011403; 20150024952; 20150025123; 20150044288; 20150086543; 20150086989; 20150087529; 20150100244; 20150104440; 20150126374; 20150141362; 20150152505; 20150191796; 20150202261; 20150203850; 20150233933; 20150275216; 20150275298; 20150284783; 20150299119; 20150299322; 20150299810; 20150307947; 20150309026; 20150322511; 20150322512; 20150322513, WO1995010271; WO1996033703; WO1998039429; WO2000073509; WO2001021836; WO2001030972; WO2001055449; WO2001062927; WO2001071027; WO2001090304; WO2002020754; WO2002026936; WO2002055705; WO2002079473; WO2002097031; WO2003042357; WO2003060069; WO2003062376; WO2003066877; WO2004016750; WO2004023973; WO2004048555; WO2004050858; WO2004050860; WO2004050900; WO2004053106; WO2004081535; WO2004089301; WO2004092338; WO2005018669; WO2005027713; WO2005040219; WO2005081943; WO2005110474; WO2005115452; WO2005118641; WO2006063356; WO2006066078; WO2007001399; WO2007109857; WO2007112330; WO2007126876; WO2008002933; WO2008019199; WO2008025069; WO2008030539; WO2008042407; WO2008062105; WO2008105886; WO2008109142; WO2008127735; WO2008130516; WO2008134729; WO2009007852; WO2009032145; WO2009032782; WO2009092133; WO2009095436; WO2009126304; WO2009126380; WO2009131712; WO2009155477; WO2010018731; WO2010033658; WO2010037001; WO2010102267; WO2010108126; WO2010141801; WO2010144358; WO2011025852; WO2011028531; WO2011091048; WO2011097218; WO2011113819; WO2011126806; WO2011130402; WO2011130624; WO2011135088; WO2012019041; WO2012024543; WO2012033537; WO2012049211; WO2012092379; WO2012115885; WO2012120374; WO2012162660; WO2012164936; WO2012178007; WO2013019623; WO2013026015; WO2013052523; WO2013055985; WO2013067451; WO2013078372; WO2013084002; WO2013089819; WO2013090186; WO2013090648; WO2013119326; WO2013126813; WO2013130161; WO2013131083; WO2013132044; WO2013151638; WO2014008426; WO2014020124; WO2014036314; WO2014043763; WO2014066848; WO2014093701; WO2014093924; WO2014130770; WO2014131845; WO2014134084; WO2014138133; WO2014144932; WO2014152211; WO2014153063; WO2014153111; WO2014173814; WO2014189787; WO2014191364; WO2014193611; WO2014204726; WO2014204728; WO2014204729; WO2015006811; WO2015018805; WO2015038924; WO2015042708; WO2015051173; WO2015051302; WO2015051304; WO2015078929; WO2015089277; WO2015089419; WO2015089465; WO2015089486; WO2015089511; WO2015138870; WO2015159292; WO2015159293; WO2015164228; WO2015168587; WO2015168621; WO2015184326; each of which is expressly incorporated herein by reference in its entirety.

### SUMMARY OF THE INVENTION

Nearly all the cells within higher organisms contain an identical DNA sequence. However, different cells which reside in different tissues may modify the DNA according to their function. Accordingly, DNA in the brain bares a different signature than DNA in the liver, for example.

Cell loss in the central nervous system (CNS) due to insult can result in chronic conditions such as in the case of multiple sclerosis (MS). Current methods for detection of cell loss in the CNS include a combination of brain imagining together with clinical symptoms. While useful, these tools may result in to the misdiagnosis of the disease, thereby affecting its treatment.

Multiple sclerosis (MS) is an autoimmune disease in which an impairment of the myelin sheath leads to reduced axonal conductivity and impaired neuronal function. Oligodendrocytes (ODCs) are the primary cells responsible for the production and deposition of myelin in the central nervous system (CNS). Both animal and human studies suggest that ODCs are the primary target for the immune attack in MS. DNA methylation is used by all cells to regulate the expression of tissue specific genes. In ODCs, the myelin oligodendrocyte glycoprotein (MOG) gene is demethylated (deMeth) while other cells maintain a methylated (Meth) form of the gene.

A unique methylation signature is found in myelin producing cells. These cells are known as oligodendrocytes (ODCs). DNA released from ODCs into the blood during CNS injury is detected using methylation specific primers and probes. Abnormal levels of ODC DNA serve as an indication of an ongoing destruction of ODCs in patients with CNS injury. DNA released from ODCs into the blood or cerebrospinal fluid during CNS injury is detected using methylation specific primers and probes.

The use of blood DNA to detect brain damage in MS has not been tested previously. This approach provides several important advancements to people with MS, as well as, those who are engaged in finding a cure for the disease. For example, by measuring active cell loss in the brain, physicians may be able to better diagnose this disease. In addition, “real time” measurement of brain damage may improve the ability to choose the right therapy option which best fits a given patient. Finally, organizations and companies that are involved in MS research would be able to better assess the efficacy of their drugs. This approach thus represents an affordable and minimally invasive tool for evaluating disease progression and testing both current and future therapies of the disease.

MOG DNA was measured in the blood of mice with experimental autoimmune encephalomyelitis (EAE) and in serum samples from relapsing remitting MS (RRMS) in human patients and healthy controls.

Methylation specific primers showed a high degree of specificity for demethylated MOG DNA. Blood from mice with EAE showed high levels of demethylated MOG DNA following MOG immunization. Analysis of sera from RRMS patients showed elevated levels of MOG DNA when compared healthy controls.

These data demonstrate, for the first time, the use of demethylated circulating DNA as a biomarker of ODC loss in MS. This approach may be used for diagnosing and improving MS health outcomes.

The experimental data show MOG DNA is differentially methylated in ODCs when compared with other tissues. ODC MOG DNA levels can be detected by methylation specific primers. Artificially deMeth DNA can be detected in the blood of DNA injected mice. EAE symptom development is associated with elevated ODC loss. Human MOG DNA is demethylated in the brain when compared with other tissues. Methylation specific primers show elevated ODC DNA in the blood of patients with RRMS. These findings demonstrate the utility of deMeth MOG DNA as a biomarker of ODC cell loss in MS. This biomarker may be used for early detection of MS, as well as for monitoring disease status and progression.

A method is provided for the detection of extrapancreatic circulating β cell, or β cell-derived DNA that is indicative of acute and chronic β cell or oligodendrocyte destruction, and thus provides an early biomarker for β cell or oligodendrocyte death in human tissues, serum and other bodily fluids, such as plasma, lymph, saliva, urine, cerebrospinal fluid, tears, and perhaps sweat. The method can identify death before the onset of symptoms of an associated disease, e.g., hyperglycemia and diabetes in case of loss of β cells, and multiple sclerosis in case of loss of oligodendrocytes. This strategy may prove useful for monitoring β cell or oligodendrocyte destruction in individuals at risk for the development of diabetes or multiple sclerosis, monitoring the progression of β cell or oligodendrocyte destruction in individuals with diabetes or multiple sclerosis, and use as a marker to guide therapy in patients with diabetes with possible ongoing β cell or oligodendrocyte destruction.

The present technology is not limited to detection, prognosis and treatment of multiple sclerosis or diabetes, and in fact is applicable to other pathology that causes apoptosis of specific cell types, such as oligodendrocytes or β cells, and resulting neuropathology or diabetes. Therefore, when considering test results, such other conditions would generally be included in the differential diagnosis. However, when a patient is tested after revealing a constellation of symptoms that clinically correlate with loss of a particular cell type, that diagnosis is likely. However, when other symptoms present, a differential diagnosis for other possible conditions is warranted, such as in the case of demethylated MOD DNA, oligodendrocytoma, Schwannoma, adrenoleukodystrophy, vanishing white matter disease, and Rubella-induced mental retardation, and in the case of demethylated insulin DNA, in the case of insulinoma, neurofibromatosis, carcinoid syndrome, multiple endocrine neoplasia, etc. For example, after ODC DNA is detected, a diagnosis of MS may be supported by determining the presence of anti-MOG antibodies or anti myelin basic protein (MBP) antibodies.

The technology relates the discovery that the presence of hypomethylated oligodendrocyte DNA in body fluids of a subject is indicative of oligodendrocyte death. Thus, in one embodiment, the invention is a method of detecting hypomethylated oligodendrocyte MOG DNA in a biological sample of a subject including the steps of: obtaining a biological sample from the subject, where the biological sample is obtained from other than myelinated tissues of the subject's central nervous system, and where the biological sample contains oligodendrocyte MOG DNA; determining the methylation status of at least one of the CpG dinucleotides in the oligodendrocyte MOG DNA, where when at least one of the CpG dinucleotides in the oligodendrocyte MOG DNA is determined to be unmethylated, the hypomethylated oligodendrocyte MOG DNA is detected.

As used herein, “hypomethylated” means that the extent of methylation of a target nucleic acid (such as genomic DNA) is lower than it could be (i.e., a DNA or DNA fragment in which many or most of the CpG dinucleotides are not methylated). By way of a non-limiting example, a hypomethylated nucleic acid is a nucleic acid that is less methylated than it could be, because less than all of the potential methylation sites of the nucleic acid are methylated. By way of another non-limiting example, a hypomethylated nucleic acid, such as in the MOG gene, is a nucleic acid that is less methylated in a cell type that expresses the nucleic acid (e.g., oligodendrocytes), as compared with a cell type that does not express the nucleic acid (e.g., liver cell). Thus, by way of one non-limiting example, a hypomethylated oligodendrocyte MOG DNA has less than all of the potential methylation sites methylated and is less methylated as compared with a liver cell MOG DNA.

In another embodiment, a method is provided for detecting oligodendrocyte death by detecting hypomethylated oligodendrocyte MOG DNA in a subject, where when at least one of the CpG dinucleotides in the oligodendrocyte MOG DNA is determined to be unmethylated, oligodendrocyte death is detected. In a further embodiment, a method is provided for measuring the level of oligodendrocyte death by detecting hypomethylated oligodendrocyte MOG DNA in a subject, where the amount of hypomethylated oligodendrocyte MOG DNA is quantified, and where a higher amount of hypomethylated oligodendrocyte MOG DNA indicates a higher level of oligodendrocyte death.

In one embodiment, a method is provided for diagnosing a subject with a disease or disorder associated with oligodendrocyte death by detecting hypomethylated oligodendrocyte MOG DNA, where when hypomethylated oligodendrocyte MOG DNA is detected, a disease or disorder associated with oligodendrocyte death in the subject is diagnosed. In various embodiments, the disease or disorder diagnosable by the methods of the invention includes multiple sclerosis, oligodendroglioma, Schwannoma, and other neurodegenerative diseases.

In another embodiment, a method of assessing the severity of a disease or disorder associated with oligodendrocyte death in a subject is provided by detecting hypomethylated oligodendrocyte MOG DNA, where the amount of hypomethylated oligodendrocyte MOG DNA is quantified, and where a higher quantity of hypomethylated oligodendrocyte MOG DNA indicates a greater severity of the disease or disorder in the subject. In various embodiments.

In a further embodiment, a method is provided for monitoring the progression of a disease or disorder associated with oligodendrocyte death in a subject by detecting hypomethylated oligodendrocyte MOG DNA in the subject, where when the amount of hypomethylated oligodendrocyte MOG DNA detected at a first time point is different than the amount of hypomethylated oligodendrocyte MOG DNA detected at a second time point, the difference in the amount of hypomethylated oligodendrocyte MOG DNA is an indicator of the progression of the disease or disorder associated with oligodendrocyte death in the subject.

In one embodiment, a method of monitoring the effect of a therapeutic regimen on a disease or disorder associated with oligodendrocyte death in a subject is provided by detecting hypomethylated oligodendrocyte MOG DNA in the subject, where when the amount of hypomethylated oligodendrocyte MOG DNA detected before therapeutic regimen is applied is different than the amount of hypomethylated oligodendrocyte MOG DNA detected during or after the therapeutic regimen is applied, the difference in the amount of hypomethylated oligodendrocyte MOG DNA is an indicator of the effect of the therapeutic regimen on the disease or disorder associated with oligodendrocyte death in the subject.

In one embodiment, a kit is provided for detecting hypomethylated oligodendrocyte MOG DNA in a biological sample, comprising asset of primers for selectively amplifying bisulfite-treated methylated and hypomethylated MOG DNA and probes for quantifying an amount of amplified methylated and hypomethylated MOG DNA. Primers and probes are also provided for detecting and/or quantifying hypomethylated oligodendrocyte MOG DNA.

In another embodiment, a composition comprising a biomarker is provided, where the biomarker comprises an isolated hypomethylated oligodendrocyte MOG DNA, or fragment thereof, where the isolated hypomethylated oligodendrocyte MOG DNA was isolated from a biological sample.

In a further embodiment, a composition is provided comprising an amplicon, where the amplicon was produced by PCR using at least one primer that hybridizes to a template comprising an isolated hypomethylated oligodendrocyte MOG DNA, or fragment thereof, where the isolated hypomethylated oligodendrocyte MOG DNA was isolated from a biological sample.

The presence of hypomethylated oligodendrocyte DNA, and preferably DNA corresponding to the MOG gene from those cells, outside of the tissues which normally contained myelinated neurons is indicative of oligodendrocyte death. For example, cerebrospinal fluid, plasma, serum, urine, saliva, and lymphatic fluid typically do not contain DNA corresponding to the MOG gene, and therefore these fluids may be collected and tested, with a very low threshold for normal individuals, and a higher level in patients with certain kinds of neuropathology. Thus, compositions and methods are provided that may be useful for assessing the extent of methylation of oligodendrocyte DNA, for detecting the presence of hypomethylated oligodendrocyte DNA as an indicator of oligodendrocyte death, for assessing the level of hypomethylated oligodendrocyte DNA as a measure of oligodendrocyte death, for diagnosing a disease or disorder associated with oligodendrocyte death, for monitoring the progression of a disease or disorder associated with oligodendrocyte death, for assessing the severity of a disease or disorder associated with oligodendrocyte death, for selecting a treatment regimen to treat a disease or disorder associated with oligodendrocyte death, and for monitoring the effect of a treatment of a disease or disorder associated with oligodendrocyte death.

It is an advantage that oligodendrocyte death can be detected non-invasively and earlier in the pathological process than other available methods for detecting diseases and disorders associated with oligodendrocyte death, thereby allowing for earlier diagnosis and therapeutic intervention of the pathologic process.

In one embodiment, the presence of hypomethylated oligodendrocyte specific DNA subject is detected in a biological sample obtained from the subject. In some embodiments, the biological sample is a bodily fluid. In certain embodiments, the biological sample is blood, serum, or plasma. Cerebrospinal fluid (CSF) is a privileged composition, and will likely contain higher levels of MOG gene DNA, and different proportions of hypometheylated and methylated corresponding DNA. However, as available, CSF this may be a preferred source of sample. Urine and saliva are also possible sources of the DNA sample.

In one embodiment, the hypomethylated oligodendrocyte DNA is at least some portion of the MOG gene DNA. In various embodiments, the hypomethylated MOG DNA is hypomethylated within at least one of a regulatory region, an intron, an exon, a non-coding region, or a coding region.

In various embodiments, the extent of methylation is assessed using methylation-specific PCR, a methylation-specific DNA microarray, bisulfite sequencing, pyrosequencing of bisulfite treated DNA, or combinations thereof. Information obtained (e.g., methylation status) can be used alone, or in combination with other information (e.g., disease status, disease history, vital signs, blood chemistry, etc.) from the subject or from the biological sample obtained from the subject.

In one embodiment, the detected hypomethylated oligodendrocyte DNA is at least some fragment of the MOG gene. In various embodiments, the detected hypomethylated MOG DNA is hypomethylated within at least one of a regulatory region, an intron, an exon, a non-coding region, or a coding region. In some embodiments, the extent of methylation of the detected hypomethylated oligodendrocyte MOG gene DNA is compared with the extent of methylation of the MOG gene DNA of a comparator cell type which does not express MOG. Non-limiting examples of comparator cell types useful in the methods of the invention include liver cells and kidney cells. In various embodiments, the hypomethylated oligodendrocyte DNA is detected using methylation-specific PCR, a methylation-specific DNA microarray, bisulfite sequencing, pyrosequencing of bisulfite treated DNA, or combinations thereof. In one embodiment, the biological sample is a bodily fluid. In various embodiments, the biological sample is at least one of plasma, serum or blood. In some embodiments, the amount of hypomethylated oligodendrocyte DNA detected is compared with a comparator, such as a negative control, a positive control, an expected normal background value of the subject, a historical normal background value of the subject, an expected normal background value of a population that the subject is a member of, or a historical normal background value of a population that the subject is a member of. Information obtained from the methods of the invention described herein (e.g., methylation status) can be used alone, or in combination with other information (e.g., disease status, disease history, vital signs, blood chemistry, etc.) from the subject or from the biological sample obtained from the subject.

Information obtained from the various methods can be stored in a computerized database associated with an automated processor (microprocessor) run in accordance with computer readable instructions stored on a non-transitory computer readable medium, that can be used for the analysis, diagnosis, prognosis, monitoring, assessment, treatment planning, treatment selection and treatment modification of diseases and disorders associated with oligodendrocyte death. Thus, such databases and their methods of use are encompassed, as well as non-transitory computer readable media containing instructions for controlling an automated processor to perform the various methods of the invention, data analysis, and produce outputs.

A biological sample can be obtained by appropriate methods, such as, by way of example, biopsy or fluid draw. In certain embodiments, a biological sample containing genomic DNA is used. The biological sample can be used as the test sample; alternatively, the biological sample can be processed to enhance access to nucleic acids (e.g., nucleic acids comprising methylated or unmethylated nucleotides), or copies of nucleic acids (e.g., copies of nucleic acids comprising methylated or unmethylated nucleotides), and the processed biological sample can then be used as the test sample. For example, in various embodiments, nucleic acid is prepared from a biological sample. Alternatively, or in addition, an amplification method can be used to amplify nucleic acids comprising all or a fragment of the nucleic acid in a biological sample, for use as the test sample in the assessment for the presence or absence of methylation.

There are many methods known in the art for the determination of the methylation status of a target nucleic acid and new methods are continually reported. In some embodiments, hybridization methods, such as Southern analysis, can be used (see Current Protocols in Molecular Biology, 2012, Ausubel, F. et al., eds., John Wiley & Sons, including all supplements). For example, methylation-specific restriction enzymes can be used to digest DNA, cleaving at specific sites depending upon methylation status, followed by hybridization with a nucleic acid probe. A “nucleic acid probe,” as used herein, can be a DNA probe or an RNA probe; the nucleic acid probe can contain at least one polymorphism of interest, as described herein. The probe can be, for example, the gene, a gene fragment (e.g., one or more exons), a vector comprising the gene, a probe or primer, etc. For representative examples of use of nucleic acid probes, see, for example, U.S. Pat. Nos. 5,288,611 and 4,851,330.

A preferred probe for detecting DNA is a labeled nucleic acid probe capable of hybridizing to target DNA. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target DNA. The hybridization sample is maintained under conditions which are sufficient to allow specific hybridization of the nucleic acid probe to DNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. More than one nucleic acid probe can also be used concurrently in this method. Specific hybridization of any one of the nucleic acid probes is indicative of the presence of the target DNA of interest.

In another embodiment, analysis by methylation sensitive restriction enzymes can be used to detect the methylation status of a target nucleic acid, if the methylation status results in the creation or elimination of a restriction site. A sample containing nucleic acid from the subject is used. RFLP analysis is conducted as described (see Current Protocols in Molecular Biology, supra). The digestion pattern of the relevant fragments indicates the presence or absence of methylation.

Various methods are available for determining the methylation status of a target nucleic acid. (See, for example, Rapley and Harbron, 2011, Molecular Analysis and Genome Discovery, John Wiley & Sons; Tollefsbol, 2010, Handbook of Epigenetics: The New Molecular and Medical Genetics, Academic Press). For example, direct sequence analysis can be used in the methods of the invention to detect the methylation status of a target nucleic acid. For example, bisulfite-treated DNA utilizing PCR and standard dideoxynucleotide DNA sequencing can directly determine nucleotides that are resistant to bisulfite conversion. (See, for example, Frommer et al., 1992, PNAS 89:1827-1831). Briefly, in an example direct sequencing method, primers are designed that are strand-specific as well as bisulfite-specific (e.g., primers containing non-CpG cytosines so that they are not complementary to non-bisulfite-treated DNA), flanking the potential methylation site. Such primers will amplify both methylated and unmethylated sequences. Pyrosequencing can also be used in the methods of the invention to detect the methylation status of a target nucleic acid. Briefly, in an example pyrosequencing method, following PCR amplification of the region of interest, pyrosequencing is used to determine the bisulfite-converted sequence of specific CpG dinucleotide sites in the target nucleic. (See, for example, Tost et al., 2003, BioTechniques 35:152-156; Wong et al., 2006, 41:734-739).

A microarray methylation assay can also be used to detect the methylation status of a target nucleic acid. Briefly, target nucleic acids are treated with bisulfite, amplified, hybridized to probes, labeled and detected. (See, for example, Wang and Petronis, 2008, DNA Methylation Microarrays: Experimental Design and Statistical Analysis; Weisenberger et al., 2008, Comprehensive DNA Methylation Analysis on the Illumina Infinium Assay Platform). For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854, WO 90/15070, and WO 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., and U.S. Pat. No. 5,424,186. Techniques for the synthesis of these arrays using mechanical synthesis methods are described in, e.g., U.S. Pat. No. 5,384,261.

Methylation specific PCR can also be used to detect the methylation status of a target nucleic acid. Briefly, sets of PCR primers are designed that will hybridize specifically to either methylated nucleotides or unmethylated nucleotides, after their modification by bisulfite treatment. (See, for example, Yuryev, 2007, PCR Primer Design, Volume 402, Chapter 19, Humana Press; Esteller, 2005, DNA Methylation: Approaches, Methods, and Applications, CRC Press). Non-limiting examples of primers useful in the methods of the invention included the primers exemplified by SEQ ID NOS: 003, 006 or 009, 012 for human, and SEQ ID NOS: 015, 018 or 021, 024 for mouse. The PCR process is well known in the art (U.S. Pat. No. 4,683,195, No. 4,683,202, and No. 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured from the target sequence by heating, and the process is repeated. Successive cycling of this procedure on both DNA strands provides exponential amplification of the region flanked by the primers.

Amplification may then be performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tm) for the amplification step using the set of primers, which is in the range of about 55° C. to about 70° C. Preferably, the Tm for the amplification step is in the range of about 59° C. to about 72° C. Most preferably, the Tm for the amplification step is about 60° C.

Typical hybridization and washing stringency conditions depend in part on the size (i.e., number of nucleotides in length) of the DNA or the oligonucleotide probe, the base composition and monovalent and divalent cation concentrations (Ausubel et al., 1994, eds Current Protocols in Molecular Biology).

The process for determining the quantitative and qualitative profile may provide real-time DNA amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes is the Tm, which is in the range of about 65° C. to 75° C. Preferably, the Tm for any one of the hydrolysis-probes is in the range of about 67° C. to about 70° C. Most preferably, the Tm applied for any one of the hydrolysis-probes of the present invention is about 67° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

Preferably, a primer nucleotide sequence is sufficiently complementary to hybridize to a nucleic acid sequence of about 12 to 25 nucleotides. More preferably, the primer differs by no more than 1 , 2, or 3 nucleotides from the target flanking nucleotide sequence In another aspect, the length of the primer can vary in length, preferably about 15 to 28 nucleotides in length (e.g., 15, 16, 17, 18, 19, 20, 21 , 22, 23, 24, 25, 26, 27, or 28 nucleotides in length).

A target nucleic acid, and PCR or other appropriate methods can be used to amplify all or a fragment of the nucleic acid, and/or its flanking sequences, if desired. The methylation status of the nucleic acid, or a fragment thereof (e.g., one or more exons, one or more introns, one or more intragenic regions, one or more regulatory regions, etc.), is determined, using methods elsewhere described herein or otherwise known in the art. The technique used to determine the methylation status of the target nucleic acid can vary in the methods of the invention, so long as the methylation status of the target nucleic acid is determined. In various embodiments of the invention, the methylation status of a target nucleic acid is compared with the methylation status of a comparator nucleic acid.

The probes and primers can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of P, P, S or H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the biological sample using known techniques. The nucleic acid can be double-stranded or single-stranded (i.e., a sense or an antisense single strand) and can be complementary to a nucleic acid encoding a polypeptide. The nucleic acid content may also be a DNA extraction performed on a fresh or fixed biological sample.

Routine methods also can be used to extract genomic DNA from a biological sample, including, for example, phenol extraction. Alternatively, genomic DNA can be extracted with kits such as the QIAamp™. Tissue Kit (Qiagen, Chatsworth, Calif.), the Wizard™ Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

The invention also includes compositions comprising amplicons produced by the methods described elsewhere herein using as a template the hypomethylated oligodendrocyte DNA comprising at least some portion of MOG gene DNA, which was isolated from a biological sample. In some embodiments, the hypomethylated oligodendrocyte DNA used as a template to produce the amplicons is treated with bisulfite. In some embodiments, the hypomethylated oligodendrocyte MOG DNA used as template to produce the amplicons is unmethylated on at least one of the CpG dinucleotides at oligodendrocyte-specific nucleotide positions of the human MOG gene. In some embodiments, the amplicons of the invention are produced in PCR reaction using at least one of the primers exemplified by SEQ ID NOS: 001, 002 and 004, 005; or 007, 008 and 010, 011 for human and SEQ ID NOS: 013, 014 and 016, 017; or 019, 020 and 022, 023 for mouse.

The present invention also pertains to kits useful in the methods of the invention described elsewhere herein. Such kits comprise components useful in any of the methods described herein, including for example, hybridization probes or primers (e.g., labeled probes or primers), reagents for detection of labeled molecules, restriction enzymes, allele-specific oligonucleotides, means for amplification of a subject's nucleic acid (as described elsewhere herein), means for analyzing a subject's nucleic acid (as described elsewhere herein), negative comparator standards, positive comparator standards, and instructional materials. For example, in one embodiment, the kit comprises components useful for analysis of the methylation status of nucleic acids in a biological sample obtained from a subject. A kit may also include instructional materials describing the use of the reagents and devices. A kit may also be associated with computer readable instructions for controlling an automated apparatus to perform the methods and analysis according to various teachings hereof.

A variety of kits having different components are contemplated. Generally, the kit comprises a component for detecting or quantifying methylation status of a nucleic acid obtained from the subject. In another embodiment, the kit comprises a component for collecting a biological sample, such as bodily fluid, from the subject. In another embodiment, the kit comprises instructions for use of the kit contents.

In one embodiment, the kit comprises a means to detect the methylation status of a hypomethylated oligodendrocyte DNA. In another embodiment, the kit comprises a means to quantify the level of hypomethylated oligodendrocyte DNA present in the subject (as described elsewhere herein).

In various embodiments, methods of the invention assesses the presence of β cell or oligodendrocyte-derived DNA that is released upon β cell or oligodendrocyte death by using a quantitative probe technology in a traditional PCR assay. The expression of insulin is epigenetically controlled by DNA methylation. By using probes, the method permits one to identify demethylated insulin or MOG DNA patterns that are uniquely or quasi-uniquely present only in β cells, distinguished from methylated insulin patterns as are present in other body cells.oligodendrocytes. Therefore, the method provides a bioassay for detecting β cell loss in diabetes or oligodendrocyte loss in neurodegenerative disease such as multiple sclerosis, to provide a method capable of improving disease diagnosis, allowing for disease staging, and providing a better evaluation of clinical treatment efficacy. In various embodiments of the invention detects β cell loss associated with T1D, T2D, or gestational diabetes, or any combination thereof.

The method as disclosed herein according to a first embodiment uses a stepwise detection and analysis of oligodendrocyte and non-oligodendrocyte derived MOG DNA. The key principle behind the method is the existence of unique DNA methylation patterns in the oligodendrocytes that are absent from other cells in the body. That is, the oligodendrocyte DNA methylation pattern associated with the MOG gene is reasonably unique, and the level of oligodendrocyte-origin MOG gene DNA in the serum and other body fluids is altered by oligodendrocyte death or pathology.

The method as disclosed herein according to an embodiment uses a stepwise detection and analysis of β cell and non-β cell derived insulin DNA. The key principle behind the method is the existence of unique DNA methylation patterns in the β cells that are absent from other cells in the body. That is, the islet β cell DNA methylation pattern associated with the insulin gene is reasonably unique, and the level of islet β cell-origin insulin gene DNA in the serum and other body fluids is altered by islet β cell death or pathology.

By first conducting a bisulfate conversion of DNA extracted from a bodily fluid of an individual, it becomes possible to quantify the relative abundance of β cell insulin or oligodendrocyte MOG DNA, respectively, in the circulation or other body fluids, and hence whether that individual is experiencing β cell or oligodendrocyte loss. Of course, loss of other cells which have distinctive gene methylation patterns may also be determined in a similar manner.

According to another embodiment, a pattern of gene methylation from a particular cell, even absent uniqueness of demethylation of any particular gene, may be analyzed, by looking for quantitative correlations of demethylated gene DNA in the body fluid. For example, if one considers that certain demethylated genes may be rare in the organism as a whole, but not unique for any particular cell type, when taken as a group, concentrations of a set of rare demethylated gene DNA may provide a reliable indication of a particular cell type of origin, using statistical methods such as principal component analysis. See, US Patent Application Nos. 20090047269; 20090123374; 20090234202; 20090264306; 20100009905; 20100086523; 20100273258; 20110003707; 20110053164; 20110166059; 20120232016; 20130035374; 20130035864; 20130052238; 20130136722; 20130218474; 20130260390; 20130261009; 20130305415; 20140031308; 20140127716; 20140137274; 20140141986; 20140147932; 20140148350; 20140170663; 20140189903; 20140199273; 20140271455; 20140274767; 20140315301; 20150052630; 20150099811; 20150119350; 20150164952; 20150285802; 20150292029; 20150298091; 20150299791; 20150301055; and PCT Publication Nos. WO2007050706; WO2007112330; WO2008095050; WO2009026152; WO2009126380; WO2010020787; WO2010029167; WO2010123354; WO2010124207; WO2010144358; WO2011133288; WO2011133935; WO2011141711; WO2011157995; WO2012024543; WO2012033537; WO2012047899; WO2012071469; WO2012115885; WO2012122236; WO2013017701; WO2013091074; WO2013148147; WO2013159103; WO2014071281; WO2014081987; WO2014082067; WO2014094043; WO2014133194; WO2014174470; WO2014183122; WO2014184199; WO2014186394; WO2015006590; WO2015006645; WO2015006811; WO2015020929; WO2015048852; WO2015120382; WO2015138870; WO2015153679; WO2015164212, each of which is expressly incorporated herein by reference in its entirety. After the demethylated genes are selected that provide the highest correlation with a particular disease or disorder, these may be typically be used without revalidation across a population. Likewise, absence or low levels of demethylated genes may be indicative of absence of cell death of the particular cell type of interest. When screening a large number of genes, a gene array “chip” or digital PCR or digital droplet PCR technologies may be used. See, US Patent Application Nos. 20090155791; 20110124518; 20110165567; 20130210011; 20130323728; 20140031257; 20140080715; 20140113290; 20140178348; 20150004602; 20150004610; 20150011403; 20150307946; and PCT Publication Nos. WO2009092035; WO2012052844; WO2012054730; WO2012120374; WO2012162660; WO2013090588; WO2013135454; WO2014043763; WO2014080017; WO2014184684; WO2014189787; WO2014207170; WO2015048665; 20150307919; 20150080235, each of which is expressly incorporated herein by reference in its entirety.

A method is developed for detecting β cell or oligodendrocyte death in vivo by amplifying regions of genes that: i) are expressed in β cells or oligodendrocytes (e.g., INS or MOG); and ii) contain CpG methylation sites, and then measuring the proportion of β cell or oligodendrocyte-derived DNA in the serum or other body fluids. Generally, by using probes that are specific for DNA methylation patterns in β cells or oligodendrocytes, circulating copies of β cell or oligodendrocyte-derived demethylated DNA are detected after bisulfite treatment and PCR amplification. See, Darst R P, Pardo C E, Ai L, Brown K D, Kladde M P; “Bisulfite sequencing of DNA”, Curr Protoc Mol Biol. 2010 July; Chapter 7: Unit 7.9.1-17. doi: 10.1002/0471142727.mb0709s91; “Methylation Analysis by Bisulfite Sequencing: Chemistry, Products and Protocols from Applied Biosystems”, tools.invitrogen.com/content/sfs/manuals/cms_039258.pdf, www.methods.info/Methods/DNA_methylation/Bisulphite_sequencing.html, each of which is expressly incorporated herein by reference. The method provides a noninvasive approach for detecting β cell or oligodendrocyte death in vivo that may be used to track the progression of diabetes and guide its treatment.

It is likewise understood that specific other tissues and cell types may have distinct methylation patterns from other tissues, and therefore that a corresponding technique, using appropriate PCR primers and optionally detection probes, may be used to detect apoptosis or other DNA release from these specific tissues or cell types into body fluids.

As an alternate to serum, saliva may also contain sufficient DNA containing epigenetic DNA modifications to provide a basis for diagnosis. During cell death most of the nuclear DNA is converted into nucleosomes and oligomers (Umansky, S. R., et al. [1982], “In vivo DNA degradation of thymocytes of gamma-irradiated or hydrocortisone-treated rats”; Biochim. Biophys. Acta 655:9-17), which are finally digested by macrophages or neighboring cells. However, a portion of this degraded DNA escapes phagocytic metabolism, and can be found in the bloodstream (Lichtenstein, A. V., et al. [2001], “Circulating nucleic acids and apoptosis”; Ann NY Acad Sci, 945:239-249), and also in bodily fluids. The present invention addresses the detection of β cell or oligodendrocyte-specific epigenetic modifications that are detectable in bodily fluids such as plasma and saliva following the destruction of β cells or oligodendrocytes.

A method is provided for the sensitive and specific detection of β cell death in vivo in models of autoimmune and chemically induced diabetes in mice, in human tissues, and in serum from patients with T1D and T2D. This assay identifies a specific methylation pattern in the β cell insulin DNA. This method provides a biomarker for detecting β cell loss in prediabetic mammals during progression of diabetes.

One embodiment of the method comprises the following steps:

1) Serum/plasma, or other body fluid is collected and DNA is extracted and substantially purified. Serum is reasonably available and usable, but collection of saliva or urine may be deemed less invasive. CSF may also be examined as a source of the biological sample.

2) Purified DNA is treated with bisulfite, whereupon the bisulfite converts demethylated cytosines to uracil while sparing the methylated cytosines (see en.wikipedia.org/wiki/Bisulfite_sequencing and “Methylation Analysis by Bisulfite Sequencing: Chemistry, Products and Protocols from Applied Biosystems”, Invitrogen Corp. (2007) tools.invitrogen.com/content/sfs/manuals/cros_039258.pdf, expressly incorporated herein by reference in their entirety; see also en.wikipedia.org/wiki/DNA_methylation, expressly incorporated herein by reference) (other methylation-sensitive distinctions may be exploited to distinguished between methylated and demethylated DNA, as known in the art).

3) Circulating DNA exists in relatively low abundance. Therefore, bisulfite treated DNA is subject to a first step polymerase chain reaction (PCR). This reaction is methylation insensitive and is designed to increase the availability of DNA template. PCR products are run on a standard gel electrophoresis and purified. Since the DNA is previously bisulfate treated, there will be distinct DNA subpopulations corresponding to methylated and demethylated insulin or MOG gene DNA, for both the sense and antisense strands.

4) Purified DNA is used for a methylation sensitive reaction, that is, the reaction distinguishes between amplified DNA corresponding to methylated insulin or MOG gene DNA and demethylated insulin or MOG gene DNA (i.e., from β cells or oligodendrocytes). The reaction uses, for example, methylation sensitive probes to detect and differentiate demethylated insulin or MOG DNA from β cell or oligodendrocyte origin from methylated insulin or MOG DNA of non-β cell or oligodendrocyte origin.

Optionally, relative numbers of β cell or oligodendrocyte derived DNA are presented as “methylation index” or 2^{(methylated DNA-demethylated DNA)} or the difference between methylated DNA and demethylated DNA. Other quantitative analysis of the results, as well as historical trend analysis is possible. Further, the amount of β cell or oligodendrocyte derived DNA may be normalized on a different basis than non-β cell or oligodendrocyte derived DNA representing the insulin or MOG gene. For example, a tracer similar in characteristics to the β cell or oligodendrocyte derived DNA (but unique with respect to endogenous DNA) may be quantitatively injected into a patient.

5) Provide a quantitative reference for the amount of β cell or oligodendrocyte derived DNA normalized for dilution, degradation, secretion/excretion factors, etc.

It is therefore an object to provide a method for monitoring β cell or oligodendrocyte pathology, comprising: extracting and purifying DNA from a body fluid of an animal; treating the extracted purified DNA with bisulfite to convert demethylated cytosine to uracil while sparing the methylated cytosines; amplifying the bisulfite-treated DNA using polymerase chain reaction; purifying the amplified bisulfite-treated DNA; performing a methylation sensitive reaction on the purified bisulfite-treated DNA using at least two different methylation specific probes which quantitatively distinguish between demethylated insulin or MOG DNA of β cell or oligodendrocyte origin and methylated insulin or MOG DNA of non-β cell or oligodendrocyte origin; and computing a quantitative relationship between methylated insulin or MOG DNA and demethylated insulin or MOG DNA.

It is a further object to provide a method for monitoring cell death of a cell type having at least one DNA portion that has a unique DNA CpG methylation pattern as compared to other cells, which is released into body fluids upon cell death of cells of the cell type, comprising: extracting and purifying DNA that comprises the DNA portion; treating the extracted purified DNA with bisulfite to convert cytosine to uracil while sparing the CpG methylated cytosines; amplifying a region of the bisulfite-treated DNA that comprises the DNA portion by polymerase chain reaction using DNA CpG methylation pattern independent primers; determining a quantitative relationship between the DNA portion having the unique DNA CpG methylation pattern to the DNA portion lacking the unique DNA CpG methylation pattern, by employing the DNA CpG methylation pattern-specific probes; computing a difference between the DNA portion having the unique DNA CpG methylation pattern and the DNA portion lacking the unique DNA CpG methylation pattern.

Another object provides a method for monitoring β cell or oligodendrocyte death, comprising: extracting and purifying genomic DNA from a body fluid of an animal, wherein the genomic DNA comprises at least a portion of a gene that is predominantly expressed by β cells or oligodendrocytes and that contains a CpG methylation site; treating the genomic DNA with bisulfite; performing a polymerase chain reaction (PCR) with primers that flank a region of the genomic DNA that comprises the CpG methylation site; purifying the PCR products; melting the PCR products into single strands; hybridizing the single-stranded PCR products with a first oligonucleotide probe capable of hybridizing with a target sequence that comprises a site corresponding to a bisulfite-converted CpG site and a second oligonucleotide probe capable of hybridizing with a target sequence that comprises a site corresponding to a bisulfite-nonconverted CpG site, and wherein the probes each comprise a non-FRET label pair consisting of a fluorophore and a quencher, and wherein interaction of the first oligopeptide probe or second oligopeptide probe with a respective target causes the first oligopeptide probe or second oligopeptide probe to change from a first conformation to a second conformation, thereby changing the distance between the fluorophore and quencher of said label pair, and wherein in only one conformation do the fluorophore and quencher interact sufficiently to quench the fluorescence of the fluorophore by a predetermined amount; quantitatively measuring fluorescent signals emitted by the first oligopeptide probe and the second oligopeptide probe; and reporting a quantitative relationship of the fluorescent signal emitted by the first oligopeptide probe and the second oligopeptide probe, indicative of the relative amount of β cell or oligodendrocyte-derived DNA versus non-β cell or oligodendrocyte-derived DNA.

It is also an object to provide a kit for detecting β cell or oligodendrocyte-derived demethylated genomic DNA in a biological sample, wherein the kit comprises: PCR primers that flank a portion of a gene that is predominantly expressed by β cells or oligodendrocytes and contains a CpG methylation site; a first oligonucleotide probe capable of hybridizing with a first target sequence on a PCR product made using the PCR primers, wherein the first target sequence corresponds to at least one bisulfite-converted CpG site of the portion of the gene; and a second oligonucleotide probe capable of hybridizing with a target sequence on a PCR product made using the PCR primers of the kit, wherein the target sequence corresponds to at least one bisulfite-nonconverted CpG site of the portion of the gene, wherein the first oligopeptide probe and the first oligopeptide probe each comprise label that allows selective quantitation of the first oligopeptide probe and the second oligopeptide probe. Each probe may comprise a label pair consisting of a fluorophore and a quencher, and wherein a binding interaction of the first oligopeptide probe with the first target sequence, and the second oligopeptide probe with the second target sequence, causes a change from a first conformation to a second conformation, thereby changing an interaction between the fluorophore and quencher of said label pair, and wherein in only one conformation of the first and second conformations do the labels interact sufficiently to quench the fluorescence of the fluorophore by, e.g., at least 25 percent, for example at least 50 percent.

The probes may be conjugated to a fluorophore and/or a quencher. The fluorophore may be at least one of 6-carboxy fluorescein and tetrachlorofluorescein. The quencher may be tetramethylrhodamine. The probe may employ a fluorescent resonant energy transfer (FRET) interaction between the fluorophore and quencher, wherein the fluorophore and quencher are selectively separated in dependence on a binding of the probe to a respective target. The probe may also employ a non-FRET interaction between the fluorophore and quencher, wherein the fluorophore and quencher have an interaction based on a conformation of the probe, and in which the conformation is selectively dependent on a binding of the probe to a respective target.

The methylation sensitive reaction may comprises quantitatively determining a release of a fluorophore from a probe bound to the purified bisulfite-treated DNA.

The DNA portion having the unique DNA CpG methylation pattern may comprise an insulin or MOG gene from a pancreatic β cell or an oligodendrocyte. The body fluid may be, for example, blood, blood plasma, blood serum, urine, saliva, cerebrospinal fluid, or tears.

### BRIEF DESCRIPTION OF THE FIGURES

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings, in which

FIG. 1 sets forth the overall procedure for detecting circulating β cell DNA. Following isolation, the DNA is bisulfite treated. A first step PCR reaction is designed to increase available template and improve DNA detection. Products are tested with hypermethylation and hypomethylation specific probes;

FIG. 2A shows the results of testing logarithmic serial dilutions of synthetic hypomethylated and hypermethylated DNA;

FIG. 2B shows that Log₁₀ transformation of demethylation index measurements show a non-linear fit;

FIG. 2C shows the increase in specificity and sensitivity of the assay used in the present method;

FIG. 3A demonstrates improved glucose levels in patients with long-standing Type 2 diabetes;

FIG. 3B shows that the probes according to the present technology reveal a significant increase in demethylated β cell DNA in the serum of the patients with long-standing Type 2 diabetes;

FIG. 3C shows that nested PCR using primers generally according to Akirav (2011) fail to reveal a significant increase in demethylated β cell DNA in the serum of the patients with long-standing Type 2 diabetes;

FIG. 4A shows the ability of the assay used in the present method to detect elevated demethylated DNA levels in the ob/ob leptin deficient mouse model of Type 2 diabetes;

FIG. 4B correlates the levels shown in FIG. 4A with elevated body weight;

FIG. 4C correlates the levels shown in FIG. 4A with increased glucose levels;

FIG. 5 shows a schematic drawing demonstrating epigenetic gene regulation by methylation; methylated genes have suppressed gene expression while demethylated genes can be expressed;

FIG. 6 shows the schematic for the use of differentially methylated DNA as a biomarker for cell loss;

FIG. 7 shows a laboratory workflow schematic for conducting the process;

FIG. 8 shows fluorescent micrographs of a spontaneous model of human type 1 diabetes in Non-Obese Diabetic (NOD) mouse, stating for DAPI, Insulin and CD31;

FIGS. 9A and 9B show graphs of blood glucose vs. time and demethylation index (DMI) vs. time for prediabetic NOD mice, showing a progressive loss of glucose tolerance;

FIG. 10A shows that methylation patterns are highly conserved in human and mouse insulin;

FIGS. 10B and 10C show that human kidney and human islet beta cells show differential methylation patterns;

FIG. 11A shows that the demethylation index of insulin DNA from islet cells is high, and corresponds to authentic unmethylated insulin DNA;

FIG. 11B shows that β insulin DNA is enriched in human islets and in the sera of recent onset type 1 diabetic patients vs. controls;

FIG. 12 shows a graph demonstrating that β cell derived DNA is significantly higher in the sera very-recent onset T1D Patients;

FIG. 13 shows a graph of DMI levels in normal controls with BMI>25 and a patient, demonstrating use of the technology as a diagnostic or prognostic tool;

FIG. 14A shows a schematic depiction of the assay using PCR primers. Following isolation, the DNA is bisulfite treated. A first step PCR reaction is designed to increase available template and improve DNA detection. Products are gel purified and analyzed using quantitative real time PCR;

FIG. 14B shows a schematic depiction of the assay using probes for detection. Following isolation, the DNA is bisulfite treated. A first step PCR reaction is designed to increase available template and improve DNA detection. Products are gel purified and analyzed using quantitative real time PCR products are tested with hypermethylation and hypomethylation specific probes;

FIG. 15A shows Mouse MOG-DNA from various tissues. Red arrow-methylated cytosine. Green arrow-demethylated cytosine converted to a thymine. Note a mixed population of DNA in brain tissue (red and green arrows). ODC+ fraction shows a single peak of thymine indicating demethylation of the gene;

FIG. 15B shows that human tissues containing ODCs, e.g., brain, show a marked increase in MOG DMI values as compared to other tissues, e.g., liver, based on real time PCR;

FIG. 16 shows detection of synthetic ODC MOG-DNA in the blood of mice. Mice were injected with synthetic MOG DNA and serum collected 2-4 minutes post injection. Data shows the ability of the assay to detect ODC MOG-DNA in the blood of mice which received synthetic DNA injection;

FIG. 17 shows a schematic representation of assay methods according to the present invention, with use of hypermethylation-specific and hypomethylation-specific PCR primers;

FIG. 18 shows that a first step PCR detects MOG DNA in different mouse tissues;

FIGS. 19A-19C show methylation specific primers detect high levels of deMeth MOG DNA in the brain (FIG. 19A) and ODC positive fraction (FIG. 19B) of CNS of mice, and low levels of deMeth MOG DNA in liver tissue (FIG. 19C);

FIG. 20 shows EAE scores in rMOG35-55 immunized mice;

FIG. 21 shows DMI scores in rMOG35-55 immunized mice;

FIG. 22 shows a relative abundance of deMeth MOG DNA in human tissues;

FIG. 23 shows an evaluation of oligodendrocyte MOG-DNA in the blood of MS patients and controls.

FIG. 24 shows that human patients with RMMS show higher levels of ODC MOG

DNA than human controls;

FIGS. 25A and 25B show a schematic depiction of ODC cell loss in MS, with healthy tissue shown in FIG. 25A, and pathological tissue in FIG. 25B;

FIGS. 26A-26D show Sanger sequencing results of bisulfite treated DNA from murine tissues. The arrows point toward CpG sites where cytosines (C) are preserved in methylated samples (Liver, Kidney), or converted to thymines (T) in samples containing demethylated CpGs, leading to a mixed population of C's and T's (Brain, Spinal Cord);

FIGS. 27A and 27B show separation of O4⁺ and O4⁻ cells from digested murine brain tissue by magnetic beads;

FIG. 28 shows DNA from murine O4⁺ cells is differentially methylated in the MOG gene compared to DNA from O4⁻ cells, the SW10 Schwann cell line, and liver;

FIG. 29 shows a depiction of MOG gene region utilized for mouse methylation-specific qPCR analysis; the cytosine at by +2,553 from MOG transcription start site is incorporated into the reverse primer sequence;

FIG. 30 shows the demethylation index determined using methylation-specific primers tested using plasmids containing methylated and demethylated murine MOG DNA inserts over a wide range of serial dilutions (R²=0.987, p<0.0001);

FIG. 31 shows that methylation-specific primers were used in qPCR with bislufite treated DNA from murine liver, kidney, brain, and spinal cord;

FIG. 32 shows the demethylation index determined using methylation-specific primers in qPCR with murine O4⁺ cells, O4⁻ cells, and SW10 Schwann cells;

FIG. 33 shows a bar graph comparing results of methylation-specific primers to detect DeMeth MOG DNA in bisulfite-treated DNA from serum of mice injected with DeMeth MOG plasmid and cuprizone-treated non-injected mice, run on qPCR with methylation-specific primers; Tx vs. Ctrl p<0.012;

FIG. 34 shows a graph of bisulfite-treated DNA from serum of cuprizone-fed mice (n=12), run on qPCR with methylation-specific primers; MOG DMIs peak at Day 14 and remain elevated over baseline until Day 35;

FIGS. 35A and 35B show brain sections from cuprizone-fed and control mice stained for myelin using Luxol fast-blue; arrows indicate region of native myelination;

FIG. 36 depicts the MOG gene region utilized for human methylation-specific qPCR analysis; cytosines at bps +2,156 and +2,181 from MOG transcription start site incorporated into the reverse primer sequence;

FIGS. 37A and 37B show Sanger sequencing results of bisulfite treated DNA from liver and brain human tissues, respectively, in which the -most arrows point toward CpG sites where cytosines (C) are preserved in methylated sample (Liver), or converted to thymines (T) in sample containing demethylated CpGs, leading to a mixed population of C's and T's (Brain); red arrows (left and middle) indicate CpGs incorporated into reverse primers;

FIG. 38 shows a logarithmic graph of methylation-specific primers tested using plasmids containing methylated and demethylated human MOG DNA inserts over a wide range of serial dilutions (R²=0.992, p<0.0001);

FIG. 39 shows a bar graph of methylation-specific primers used in qPCR with bisulfite treated DNA from human liver, brain, and Meth and Demeth MOG plasmids;

FIGS. 40A and 40B provide data plots showing that methylation-specific primers can detect elevated levels of demethylated MOG cfDNA in patients with RRMS. FIG. 40A shows

Age at diagnosis of relapsing-remitting multiple sclerosis for both disease active and inactive groups. FIG. 40B shows duration of disease of relapsing-remitting multiple sclerosis for both active and inactive groups;

FIG. 41 shows a bargraph comparing demethylation index from methylation-specific primers were used in qPCR with bisulfite treated DNA extracted from sera from Healthy Controls, Inactive and Active RRMS patients; ANOVA p<0.029, Inactive vs. Active p<0.05;

FIG. 42 shows an ROC analysis of samples showed an AUC of 0.7475 with 95% confidence interval of 0.59-0.9 (statistical significance p<0.007);

FIG. 43 shows a graph, generated using a total insulin probe and hypomethylated specific insulin probes in a ddPCR reaction using RainDance Technologies. Neg: Non-demethylated DNA originated from non-beta cells. Pos: DeMethylated DNA from beta cells. Template used are primary islet preparations from humans; and

FIGS. 44A and 44B how ddPCR analysis of two patients with recent onset T1D; Both patients were positive for C-peptide indicated the presence of residual beta cells.

### DETAILED DESCRIPTION

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

In various embodiments, the present technology substantially isolates nucleic acids from a sample of body fluid, for example blood plasma, urine, saliva, cerebrospinal fluid, lymph fluid, synovial fluid, mucous, sweat, or tears, for example.

Various DNA extraction, isolation and purification technologies can be used, for example as taught in U.S. Pat. Nos. 4,935,342, 5,990,301, 6,020,124, 7,241,596, 6,485,903, 6,214,979, Re. 39,920 each of which is expressly incorporated herein by reference in its entirety.

An anion exchange material may be selected and employed which effectively adsorbs the target nucleic acids or protein complexes thereof. For example, commercially available anion exchange materials may be employed. Either strong or weak anion exchangers may be employed. A preferred weak exchanger can be one in which primary, secondary, or tertiary amine groups (i.e., protonatable amines) provide the exchange sites. The strong base anion exchanger has quaternary ammonium groups (i.e., not protonatable and always positively charged) as the exchange sites. Both exchangers can be selected in relation to their respective absorption and elution ionic strengths and/or pH for the nucleic acid being separated. Purification by anion exchange chromatography is described in U.S. Pat. No. 5,057,426 (see also EP 0 268 946 B1), expressly incorporated by reference herein in its entirety.

The material which is commercially available under the designation Q-Sepharose™ (GE Healthcare) is a particularly suitable. Q-Sepharose™, can be a strong anion exchanger based on a highly cross-linked, bead formed 6% agarose matrix, with a mean particle size of 90 μm. The Q-Sepharose™ can be stable in all commonly used aqueous buffers with the recommended pH of 2-12 and recommended working flow rate of 300-500 cm/h. In other preferred embodiments, the anion-exchange medium can be selected from sepharose-based quaternary ammonium anion exchange medium such as Q-filters or Q-resin.

The chromatographic support material for the anion charge used in the instant methods can be a modified porous inorganic material. As inorganic support materials, there may be used materials such as silica gel, diatomaceous earth, glass, aluminum oxides, titanium oxides, zirconium oxides, hydroxyapatite, and as organic support materials, such as dextran, agarose, acrylic amide, polystyrene resins, or copolymers of the monomeric building blocks of the polymers mentioned.

The nucleic acids can also be purified by anion exchange materials based on polystyrene/DVB, such as Poros 20 for medium pressure chromatography, PorosT™ 50 HQ, of the firm of BioPerseptive, Cambridge, U.S.A., or over DEAE Sepharose™, DEAE Sephadex™ of the firm of Pharmacia, Sweden; DEAE Spherodex™, DEAE Spherosil™, of the firm of Biosepra, France.

In various embodiments, the present technology substantially isolates nucleic acids from a sample of body fluid, for example blood plasma, saliva, cerebrospinal fluid, lymph fluid, synovial fluid, urine, or tears, for example.

Various DNA extraction, isolation and purification technologies can be used, for example as taught in U.S. Pat. Nos. 4,935,342, 5,990,301, 6,020,124, 7,241,596, 6,485,903, 6,214,979, Re. 39,920 each of which is expressly incorporated herein by reference in its entirety.

An anion exchange material may be selected and employed which effectively adsorbs the target nucleic acids or protein complexes thereof. For example, commercially available anion exchange materials may be employed. Either strong or weak anion exchangers may be employed. A preferred weak exchanger can be one in which primary, secondary, or tertiary amine groups (i.e., protonatable amines) provide the exchange sites. The strong base anion exchanger has quaternary ammonium groups (i.e., not protonatable and always positively charged) as the exchange sites. Both exchangers can be selected in relation to their respective absorption and elution ionic strengths and/or pH for the nucleic acid being separated. Purification by anion exchange chromatography is described in U.S. Pat. No. 5,057,426 (see also EP 0 268 946 B 1), expressly incorporated by reference herein in its entirety.

The material which is commercially available under the designation Q-Sepharose™ (GE Healthcare) is a particularly suitable. Q-Sepharose™, can be a strong anion exchanger based on a highly cross-linked, bead formed 6% agarose matrix, with a mean particle size of 90 μm. The Q-Sepharose™ can be stable in all commonly used aqueous buffers with the recommended pH of 2-12 and recommended working flow rate of 300-500 cm/h. In other preferred embodiments, the anion-exchange medium can be selected from sepharose-based quaternary ammonium anion exchange medium such as Q-filters or Q-resin.

The chromatographic support material for the anion charge used in the instant methods can be a modified porous inorganic material. As inorganic support materials, there may be used materials such as silica gel, diatomaceous earth, glass, aluminum oxides, titanium oxides, zirconium oxides, hydroxyapatite, and as organic support materials, such as dextran, agarose, acrylic amide, polystyrene resins, or copolymers of the monomeric building blocks of the polymers mentioned.

The nucleic acids can also be purified by anion exchange materials based on polystyrene/DVB, such as Poros 20 for medium pressure chromatography, Poros™ 50 HQ, of the firm of BioPerseptive, Cambridge, U.S.A., or over DEAE Sepharose™ DEAE Sephadex™ of the firm of Pharmacia, Sweden; DEAE Spherodex™, DEAE Spherosil™, of the firm of Biosepra, France.

A body fluid sample, such as blood plasma, cerebrospinal fluid, urine or saliva, containing nucleic acids or their proteinous complexes, is applied to the selected anion exchange material, and the nucleic acids or their complexes become adsorbed to the column material.

The contact and subsequent adsorption onto the resin can take place by simple mixing of the anion exchange media with the body fluid, with the optional addition of a solvent, buffer or other diluent, in a suitable sample container such as a glass or plastic tube, or vessel commonly used for handling biological specimens. This simple mixing referred to as batch processing, can be allowed to take place for a period of time sufficiently long enough to allow for binding of the nucleoprotein to the media, preferably 10 to 40 min. The media/complex can then be separated from the remainder of the sample/liquid by decanting, centrifugation, filtration or other mechanical means.

The anion exchange material can optionally be washed with an aqueous solution of a salt at which the nucleic acids remain bound to the anion exchange material, the washing being of sufficient volume and ionic strength to wash the non-binding or weakly binding components through the anion-exchange material. In some embodiments, the resin can be washed with 2×SSC (300 mM NaCl/30 mM sodium citrate (pH 7.0). Preferred ranges of the salt solutions are 300-600 nM NaCl/30 mM sodium citrate (pH 7.0). The resin may alternately be washed with 300-600 mM LiCl/10 mM NaOAc (pH 5.2).

The bound nucleic acids may then be eluted by passing an aqueous solution through the anion exchange material of increasing ionic strength to remove in succession proteins that are not bound or are weakly bound to the anion-exchange material and the nucleic acids of increasing molecular weight from the column. Both proteins and high and low molecular weight nucleic acids (as low as 10 base pairs) can be selectively eluted from the resin stepwise with the salt solution of concentrations from 300 mM to 2.0 M of NaCl and finally with 2.0 M guanidine isothiocyanate. LiCl solutions in the concentration range of 300 mM to 2.0 M of LiCl may also be used for stepwise elution.

The nucleic acids isolated may be in double-stranded or single-stranded form.

The body fluid can be pre-filtered through a membrane and supplemented with 10 mM EDTA (pH 8.0) and 10 mM Tris-HCL (pH 8.0) prior to adsorption onto the anion-exchange medium. Commercial sources for filtration devices include Pall-Filtron (Northborough, Mass.), Millipore (Bedford, Mass.), and Amicon (Danvers, Mass.). Filtration devices which may be used are, for example, a flat plate device, spiral wound cartridge, hollow fiber, tubular or single sheet device, open-channel device, etc.

The surface area of the filtration membrane used can depend on the amount of nucleic acid to be purified. The membrane may be of a low-binding material to minimize adsorptive losses and is preferably durable, cleanable, and chemically compatible with the buffers to be used. A number of suitable membranes are commercially available, including, e.g., cellulose acetate, polysulfone, polyethersulfone, and polyvinylidene difluoride. Preferably, the membrane material is polysulfone or polyethersulfone.

The body fluid, for example cerebrospinal fluid, blood plasma, urine or saliva, can be supplemented with EDTA and Tris-HCL buffer (pH 8.0) and digested with proteinases, such as for example Proteinase K, prior to adsorption onto the anion exchange medium.

The anion-exchange medium can be immobilized on an individualized carrier such as a column, cartridge or portable filtering system which can be used for transport or storage of the medium/nucleoprotein bound complex. The nucleic acid/anion exchange may be maintained in storage for up to 3 weeks.

A kit may be provided with a solid carrier capable of adsorbing the nucleic acids containing in a sample of a body fluid, for example blood plasma or saliva. The kit may also contain other components for example, reagents, in concentrated or final dilution form, chromatographic materials for the separation of the nucleic acids, aqueous solutions (buffers, optionally also in concentrated form for final adjusting by the user) or chromatographic materials for desalting nucleic acids which have been eluted with sodium chloride.

The kit may also contain additional materials for purifying nucleic acids, for example, inorganic and/or organic carriers and optionally solutions, excipients and/or accessories. Such agents are known and are commercially available. For solid phase nucleic acid isolation methods, many solid supports have been used including membrane filters, magnetic beads, metal oxides, and latex particles. Widely used solid supports include silica-based particles (see, e.g., U.S. Pub. Pat. App. 2007/0043216 (Bair Jr., et al.); U.S. Pat. No. 5,234,809 (Boom et al.); WO 95/01359 (Colpan et al.); U.S. Pat. No. 5,405,951 (Woodard); WO 95/02049 (Jones); WO 92/07863 (Qiagen GmbH), each of which is expressly incorporated herein by reference). Inorganic components of carriers may be, for example, porous or non-porous metal oxides or mixed metal oxides, e.g. aluminum oxide, titanium dioxide, iron oxide or zirconium dioxide, silica gels, materials based on glass, e.g. modified or unmodified glass particles or ground glass, quartz, zeolite or mixtures of one or more of the above-mentioned substances. On the other hand, the carrier may also contain organic ingredients which may be selected, for example, from latex particles optionally modified with functional groups, synthetic polymers such as polyethylene, polypropylene, polyvinylidene fluoride, particularly ultra-high molecular polyethylene or HD-polyethylene, or mixtures of one or more of the above-mentioned substances.

In addition, the reagent kit may also contain excipients such as, for example, a protease such as proteinase K, or enzymes and other agents for manipulating nucleic acids, e.g. at least one amplification primer, and enzymes suitable for amplifying nucleic acids, e.g. DNase, a nucleic acid polymerase and/or at least one restriction endonuclease. Alternately, a commercial polymerase chain reaction kit may be used to amplify the DNA samples, as discussed below. DNA is subject to degradation by DNases present in bodily fluids, such as saliva. Thus, in certain embodiments, it is advantageous to inhibit DNase activity to prevent or reduce the degradation of DNA so that sufficiently large sequences are available for detection.

After collection, the sample may be treated using one or more methods of inhibiting DNase activity, such as use of ethylenediaminetetraacetic acid (EDTA), guanidine-HCl, GITC (Guanidine isothiocyanate), N-lauroylsarcosine, Na-dodecylsulphate (SDS), high salt concentration and heat inactivation of DNase.

After collection, the sample may be treated with an adsorbent that traps DNA, after which the adsorbent is removed from the sample, rinsed and treated to release the trapped DNA for detection and analysis. This not only isolates DNA from the sample, but, some adsorbents, such as Hybond™.TM. N membranes (Amersham Pharmacia Biotech Ltd., Piscataway, N.J.) protect the DNA from degradation by DNase activity.

In some cases, the amount of DNA in a sample is limited. Therefore, for certain applications, sensitivity of detection may be increased by known methods.

Where DNA is present in minute amounts, larger samples can be collected and thereafter concentrated such as by butanol concentration or concentration using Sephadex™ G-25 (Pharmacia Biotech, Inc., Piscataway N.J.).

Once obtained, the bodily fluid derived DNA may be used as an alternate to serum-derived DNA as discussed below. Since the technology is ratiometric, it is dependent not on the absolute quantity of DNA available, but the proportional relationships of the methylated and unmethylated portions. In general, the disposition of these types in the various body fluids is not believed to be highly dependent on the fluid type, and calibration techniques can be used to account for persistent and predictable differences in the fluid methylated/unmethylated ratios.

In various embodiments, methylation status-specific probes are conjugated with 6-carboxyfluorescein, abbreviated as FAM, thus permitting quantitative detection. See, en.wikipedia.org/wiki/TaqMan, expressly incorporated herein by reference. Other technologies may be used in conjunction with the present method; see, U.S. Pat. Nos. 6,103,476, 8,247,171, 8,211,644, 8,133,984, 8,093,003, 8,071,734, 7,972,786, 7,968,289, 7,892,741, 7,847,076, 7,842,811 7,803,528, 7,776,529, 7,662,550, 7,632,642, 7,619,059, 7,598,390, 7,422,852, 7,413,708, 7,399,591, 7,271,265, 7,241,596, 7,183,052, 7,153,654, 7,081,336, 7,070,933, 7,015,317, 7,005,265, 6,811,973, 6,680,377, 6,649,349, 6,548,254, 6,485,903, 6,485,901, each of which is expressly incorporated in its entirety. Probes may be Fluorescent Resonance Energy Transfer (FRET) or non-FRET type. See, U.S. Pat. No. 6,150,097, expressly incorporated herein by reference.

### Example 1

### β Cell Loss in Diabetes

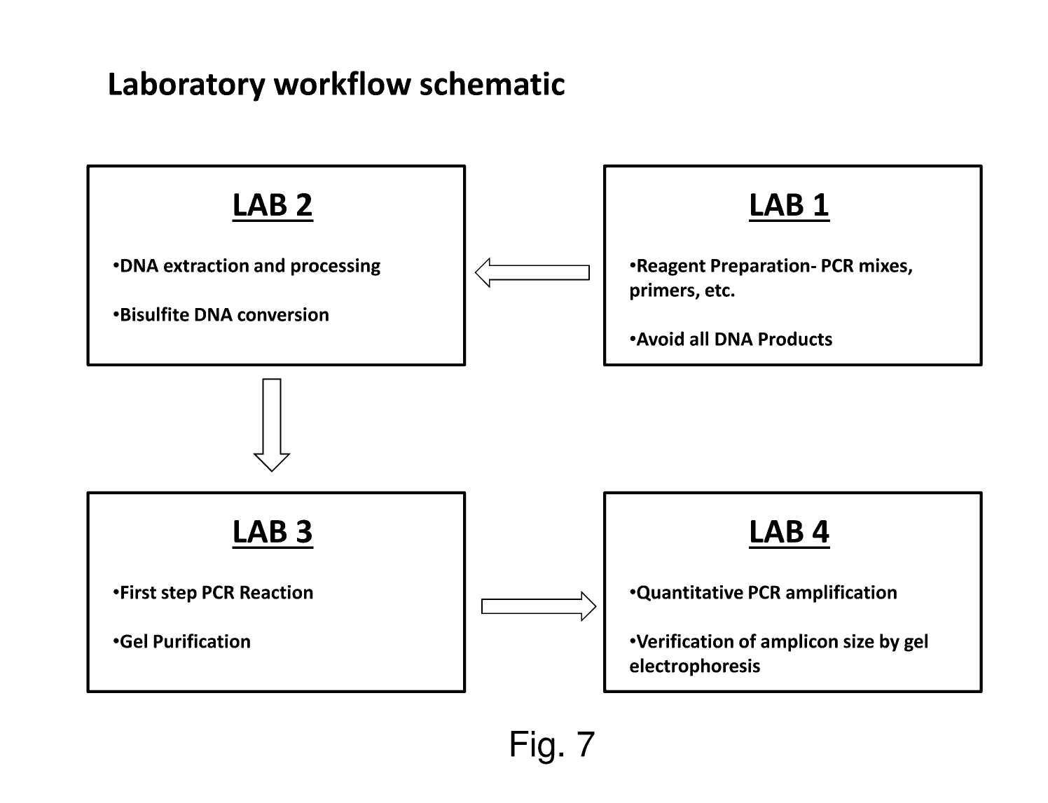

A laboratory workflow diagram is shown in FIG. 7.

Bisulfite Treatment

DNA from serum samples was purified using the Qiagen QIAamp DNA Blood Kit following the manufacturer-recommended protocol. Synthetic unmethylated and methylated DNA was purchased from Zymo research. DNA was then subjected to bisulfite treatment and purified on a DNA binding column to remove excessive bisulfite reagent using the Zymo EZ DNA Methylation Kit.

First-Step PCR and Gel Extraction.

A methylation-independent reaction was carried out to increase the DNA template for PCR analysis.

For the reaction, bisulfite-treated DNA template was added to Zymo Taq Premix. The amplification proceeded for, e.g., 50 cycles. The PCR products were excised from a 3% agarose gel. Negative controls without DNA did not yield products in the first-step reaction.

PCR products obtained using methylation-independent primers were purified using a Qiagen PCR Purification Kit.

Methylation-specific Analysis

Methylation-specific DNA probes are used for the detection of β cell derived DNA. These probes are able to quantitatively and sensitively detect circulation demethylated and methylated DNA from a β cell and a non-βcell origin, respectively. The new probes replace the previously published methylation specific primers (see Akirav E M, Lebastchi J, Galvan E M, Henegariu O, Akirav M, Ablamunits V, Lizardi P M, and Herold K C. Detection of β cell death in diabetes using differentially methylated circulating DNA. PNAS, 2011, Proceedings of the National Academy of Sciences, 2011, November:108(19018-23) hereinafter Akirav et al. (2011), and Herald et al., WO2012/178007 and US 2014/0256574, each of which is expressly incorporated herein by reference in its entirety. See also Husseiny M I, Kuroda A, Kaye A N, Nair I, Kandeel F, et al. (2012) Development of a Quantitative Methylation-Specific Polymerase Chain Reaction Method for Monitoring Beta Cell Death in Type 1 Diabetes. PLoS ONE 7(10): e47942. doi:10.1371/journal.pone.0047942; Husseiny M I, Kaye A, Zebadua E, Kandeel F, Ferreri K (2014) Tissue-Specific Methylation of Human Insulin Gene and PCR Assay for Monitoring Beta Cell Death. PLoS ONE 9(4): e94591, doi:10.1371/journal.pone.0094591; Husseiny M I, Kuroda A, Kaye A N, Nair I, Kandeel F, et al. (2012) Development of a quantitative methylation-specific polymerase chain reaction method for monitoring beta cell death in type 1 diabetes. PLoS One 7: e47942. doi: 10.1371/journal.pone.0047942; Akirav E M, Lebastchi J, Galvan E M, Henegariu O, Akirav M, et al. (2011) Detection of beta cell death in diabetes using differentially methylated circulating DNA. Proc Natl Acad Sci U S A 108: 19018-19023. doi: 10.1073/pnas.1111008108; Fisher M M, Perez Chumbiauca C N, Mather K J, Mirmira R G, Tersey S A (2013) Detection of Islet beta Cell Death in vivo by Multiplex PCR Analysis of Differentially Methylated DNA. Endocrinology; Kuroda A, Rauch T A, Todorov I, Ku H T, Al-Abdullah I H, et al. (2009) Insulin gene expression is regulated by DNA methylation. PLoS One 4: e6953. doi: 10.1371/journal.pone.0006953, each of which is expressly incorporated herein by reference), which presented with a relatively low specificity (i.e. demethylated primers detected methylated DNA and vice versa). Low specificity negatively impacts assay sensitivity by decrease detection limits of β cell derived demethylated DNA. Low DNA levels are presumably present during early β cell loss, such as prediabetes. See, U.S. Pat. No. 6,150,097, expressly incorporated herein by reference.

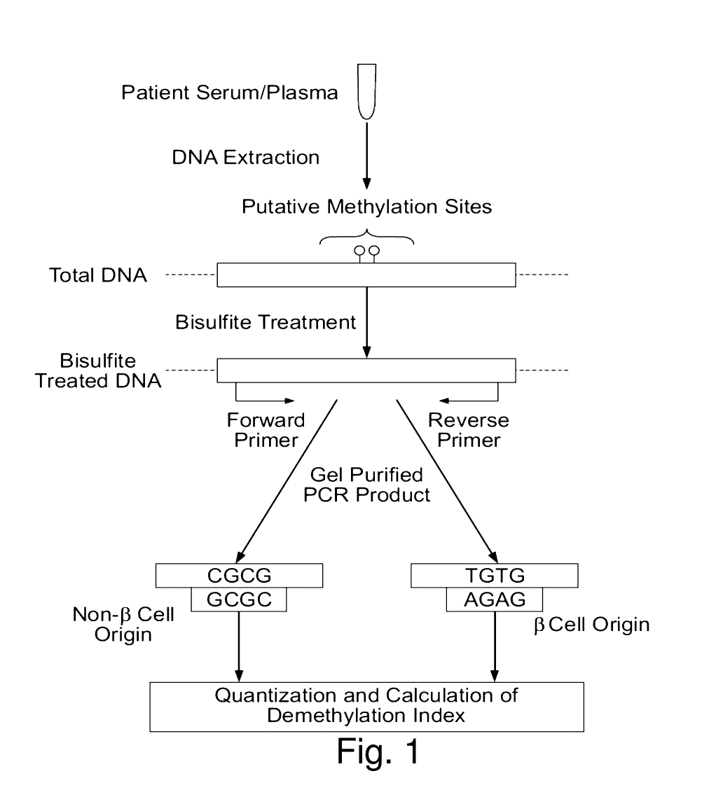

The overall procedure for the detection of circulating β cell DNA is depicted in FIG. 1. The steps leading to the use of probes are identical with those described in Akirav et al. (2011), which discloses the use of methylation-specific primers (and not probes) to detect β cell derived DNA. The primers were able to detect demethylated and methylated DNA from a β cell and a non-β cell origin, respectively. While useful, these primers had a relatively low specificity whereby demethylated primers detected methylated DNA and vice versa. Low specificity reduced assay sensitivity as it impaired the ability to detect very low levels of β cell-derived DNA, such as in the condition of early β cell loss and pre-diabetes.

DNA from serum samples was purified using the Qiagen QIAamp DNA Blood Kit following the manufacturer-recommended protocol. Synthetic unmethylated and methylated DNA was purchased from Millipore. Purified DNA was quantitated using a NanoDrop 2000 spectrophotometer. DNA was then subjected to bisulfite treatment and purified on a DNA binding column to remove excessive bisulfite reagent using the Zymo EZ DNA Methylation Kit.

The present method, in contrast, uses probe DNA that offers a significant improvement in sensitivity over the primers used in the prior art discussed above. That is, probe DNA allows for a highly specific recognition of two demethylated sites in the insulin gene. This tends to eliminate false positive readings and thus provides increased assay specificity and sensitivity. The following is used as probe for the detection of circulating DNA in the assay according to the present method:

A methylation-independent reaction was carried out to increase the DNA template for PCR analysis. For the reaction, bisulfite-treated DNA template was added to ZymoTaq™ Premix (see, www.zymoresearch.com/protein/enzymes/zymotaq-dna-polymerase, expressly incorporated herein by reference.) The following PCR primers are used to amplify the human insulin position 2122220-2121985 on chromosome 11, GRCh37.p10, October. 2012):

Forward primer:
SEQ ID NO: 001
GTGCGGTTTATATTTGGTGGAAGTT
Reverse primer:
SEQ ID NO: 002
ACAACAATAAACAATTAACTCACCCTACAA

Using the forward and reverse primers, PCR was conducted The PCR products were excised from a 3% agarose gel.

The PCR product (or amplicon) is detect by methylation status specific probes as follows:

a) Probes for the detection of methylated insulin DNA (i.e., DNA not derived from a β cell)(alternates):

SEQ ID NO: 003
ACCTCCCGACGAATCT
SEQ ID NO: 004
TACCTCTCGTCGAATCT

b) Probes for the detection of demethylated insulin DNA (i.e., DNA derived from a β cell)(alternates):

SEQ ID NO: 005
ACCTCCCAACAAATCT
SEQ ID NO: 006
TACCTCCCATCAAATCT

The methylation status-specific probes are typically conjugated with 6-carboxyfluorescein (FAM) permitting quantitative detection. Probes may be Fluorescent Resonance Energy Transfer (FRET) or non-FRET type.

c)PCR is done with an annealing temperature of 60° C. for 50 cycles and quantified using a Real Time

d)Values generated by demethylated probes are subtracted from values of methylated probes and a de

[symbol]

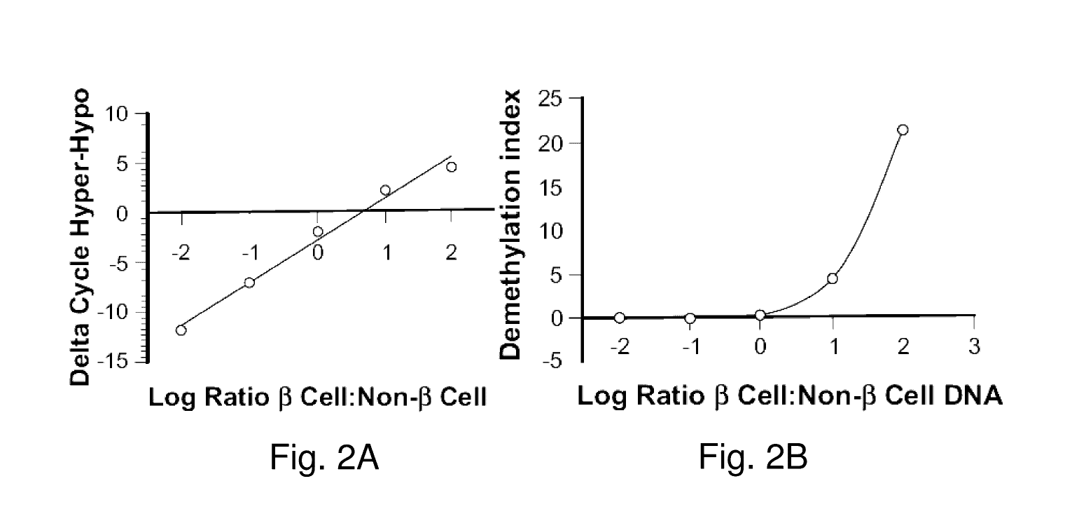

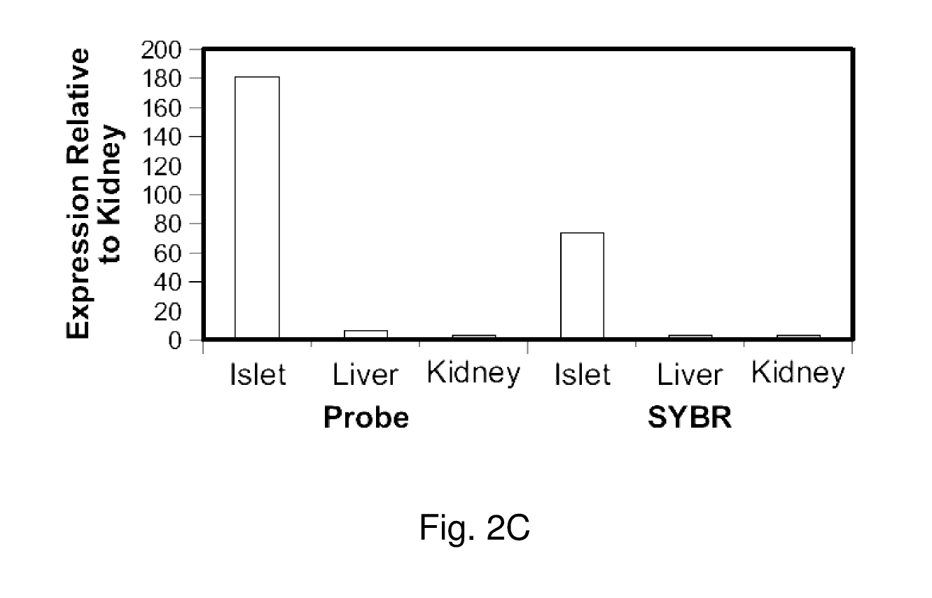

Probe testing of logarithmic serial dilutions of synthetic hypomethylated and hypermethylated DNA has shown a linear behavior (R2=0.98) of the delta between hypermethylated DNA and hypomethylated DNA (delta=hypermethylated DNA-hypomethylated DNA) over a wide range of DNA dilution (range is 4 log scale) see FIG. 2A. Log10 transformation of demethylation index measures show a nonlinear fit (R2=0.9999, DF 2) see FIG. 2B. FIG. 2C shows the specificity of the assay. The probe detects demethylated DNA at ˜180 folds in islet (where β cells reside) compared with liver and kidney which do not express insulin. In contrast, primers detect the demethylated DNA at ˜80 fold. In other words probes used according to an embodiment of the present invention are 2.25 times more specific than primers the primers used in accordance with Akirav et al. (2011).

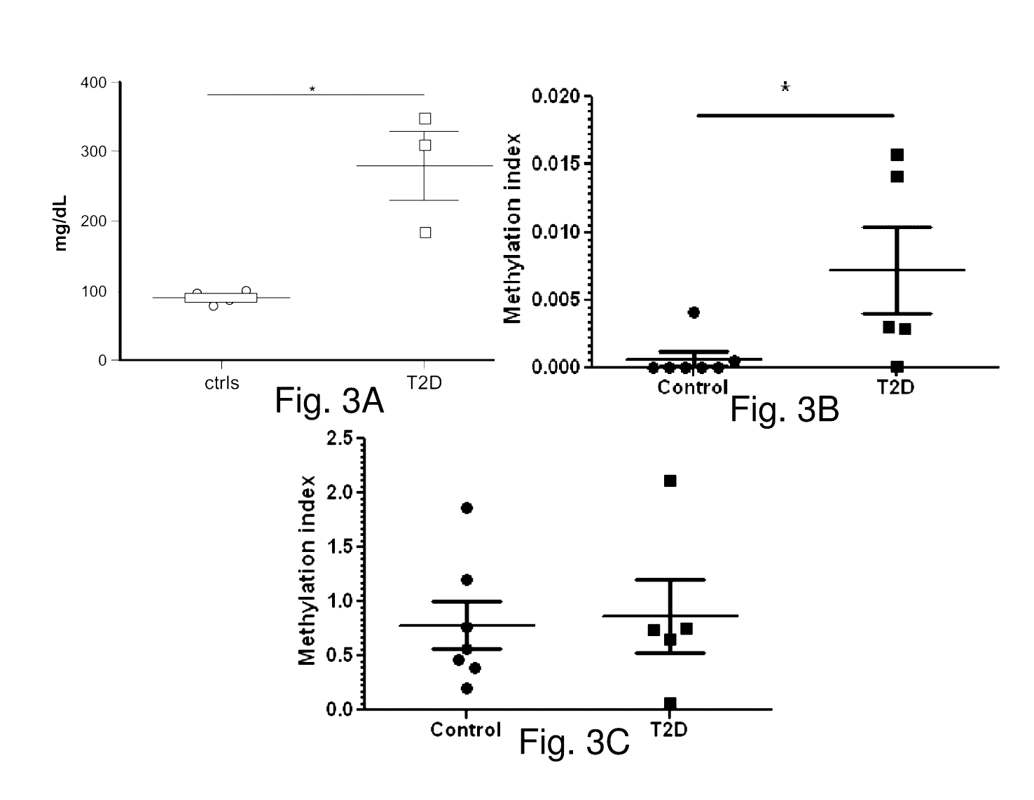

The present method extends the use of demethylated β cell derived DNA as a biomarker of Type 2 diabetes. The ability of the present assay to detect β cell loss in Type 2 diabetes is clearly shown by the experimental results obtained with the use of the present method. FIG. 3A shows impaired glucose levels in patients with long-standing Type 2 diabetes. FIG. 3B shows the increase in demethylated β cell DNA (i.e., increase in methylation index) in the serum of these patients, revealed as a significant difference (p=0.0286) from control by the use of the present probe technology. Similar results are also observed in animal models of Type 2 diabetes. FIG. 3C shows the use of primers from Akirav et al. (2011) to analyze the same sample set, and failed to detect any significant difference (p=0.87) in methylation index between control and T2D patients.

For PCR according to Akirav et al., (2011), shown in FIG. 3C was conducted for 40 cycles, with a melting temperature of 54° C., using primers as follows:

Forward primer:
SEQ ID NO: 007
TTAGGGGTTTTAAGGTAGGGTATTTGGT
Reverse primer:
SEQ ID NO: 008
ACCAAAAACAACAATAAACAATTAACTCACCCTACAA

The second step real-time methylation-specific nested PCR according to Akirav et al. (2011) was conducted with 50 cycles of amplification, and a melting temperature of 64° C., with the following primers:

Methylated forward primer:
SEQ ID NO: 009
GTGGATGCGTTTTTTGTTTTTGTTGGC
Methylated reverse primer:
SEQ ID NO: 010
CACCCTACAAATCCTCTACCTCCCG
Demethylated forward primer:
SEQ ID NO. 011
TTGTGGATGTGTTTTTTGTTTTTGTTGGT
Demethylated reverse primer:
SEQ ID NO: 012
CACCCTACAAATCCTCTACCTCCCA

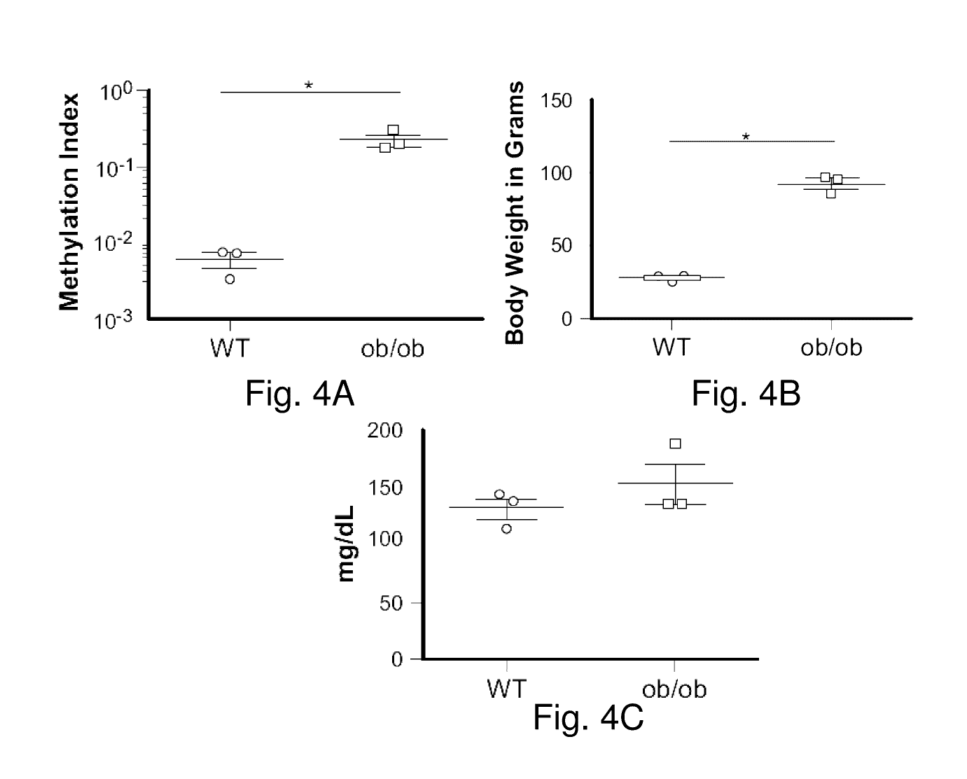

FIG. 4A shows the ability of to detect elevated demethylated DNA levels in the ob/ob leptin deficient mouse model of type 2 diabetes. These levels were correlated with elevated body weight, shown in FIG. 4B, and increased glucose levels, shown in FIG. 4C.

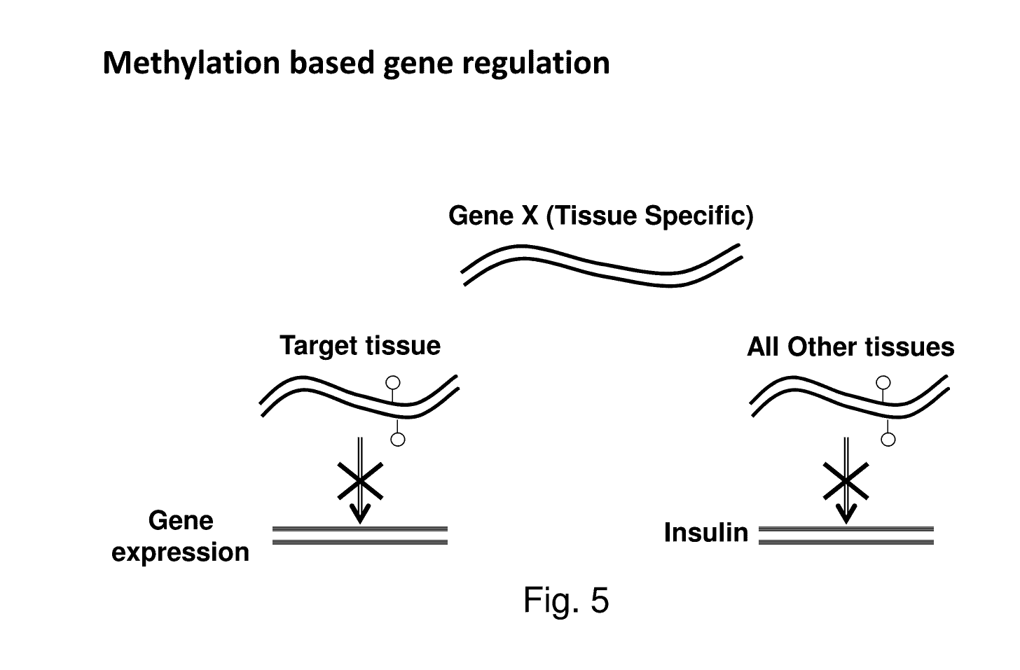

FIG. 5 shows a schematic drawing demonstrating epigenetic gene regulation by methylation; methylated genes have suppressed gene expression while demethylated genes can be expressed.

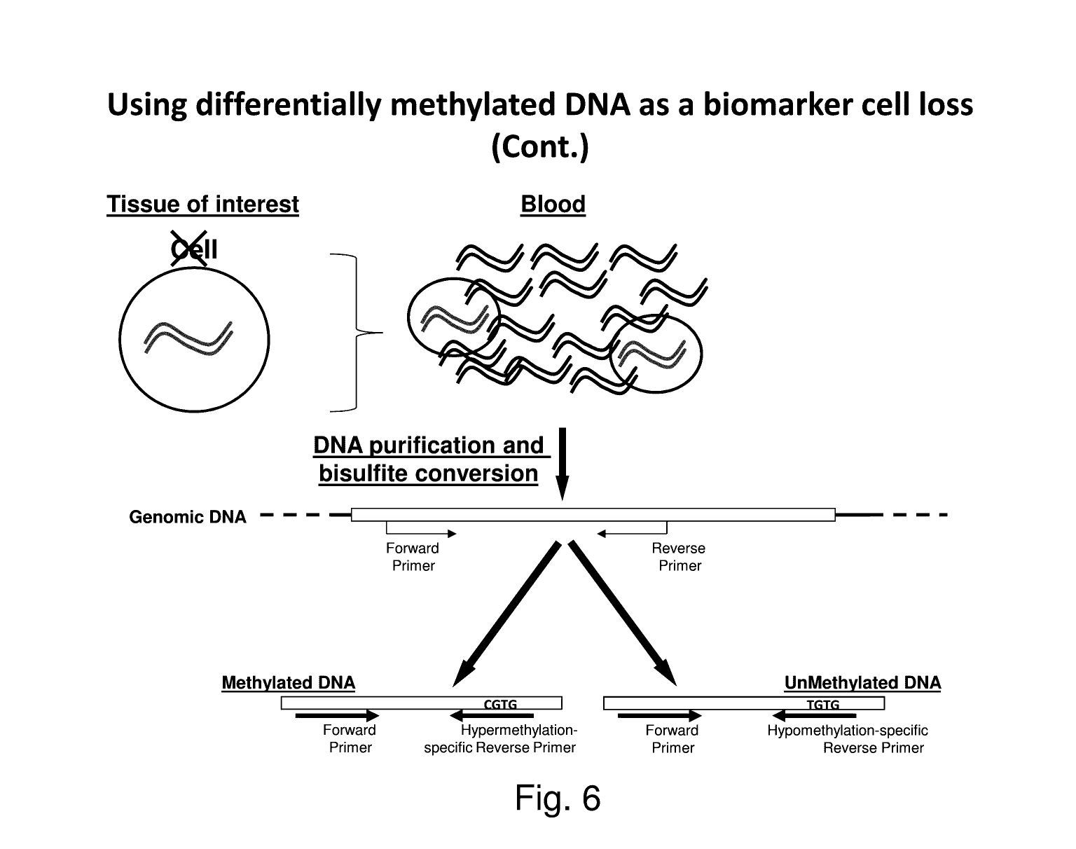

FIG. 6 shows the schematic for the use of differentially methylated DNA as a biomarker for cell loss.

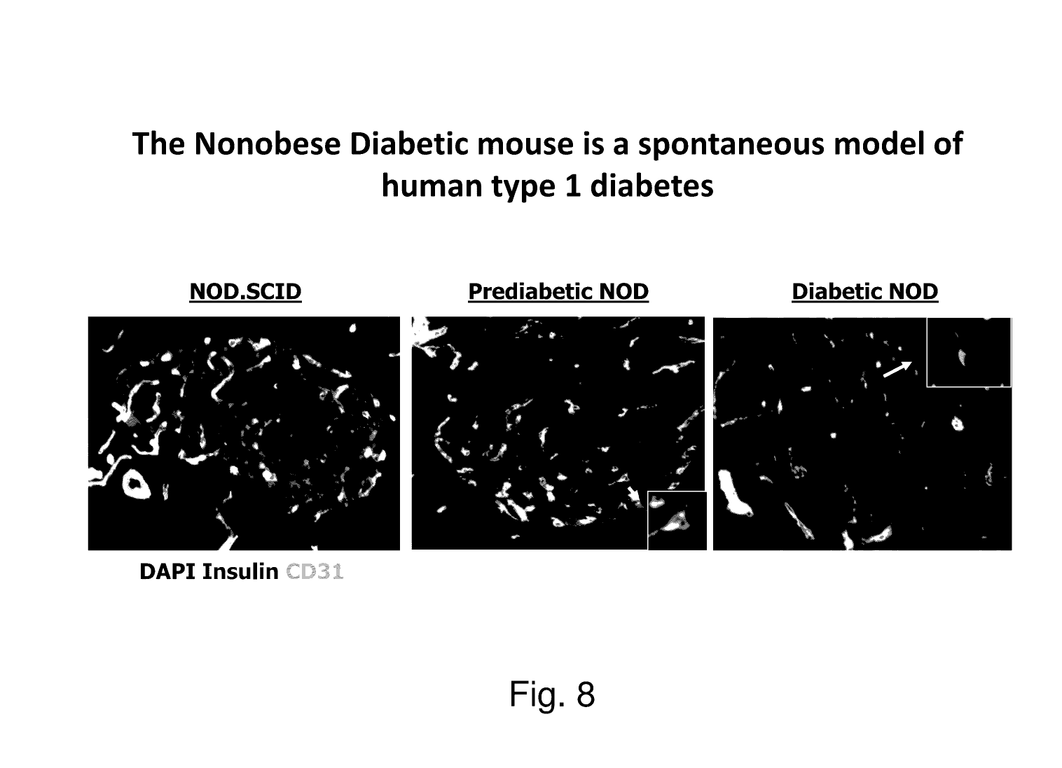

FIG. 8 shows fluorescent micrographs of a spontaneous model of human type 1 diabetes in Non-Obese Diabetic (NOD) mouse, stating for DAPI, Insulin and CD31.

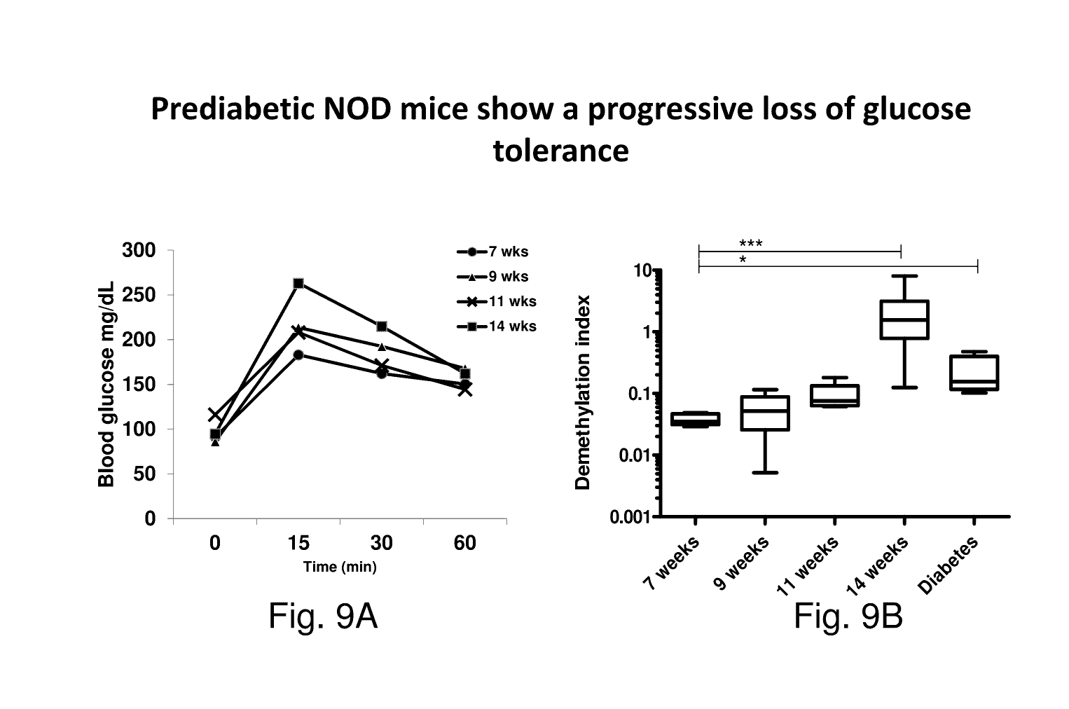

FIGS. 9A and 9B show graphs of blood glucose vs. time and demethylation index (DMI) vs. time for prediabetic NOD mice, showing a progressive loss of glucose tolerance.

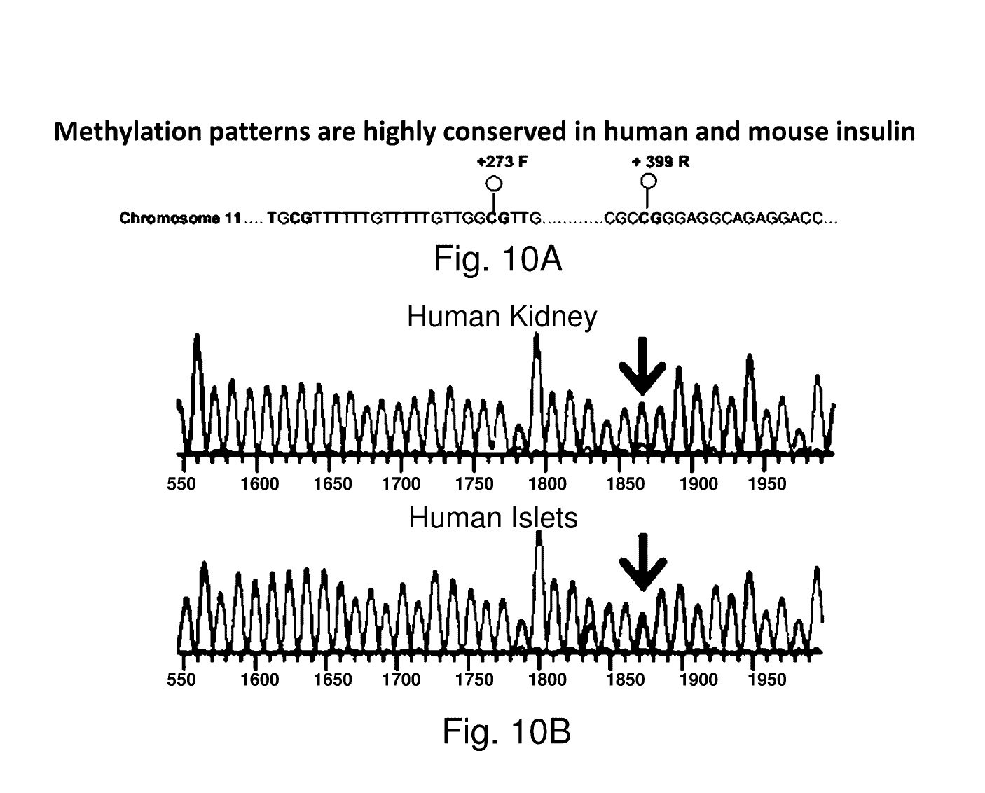

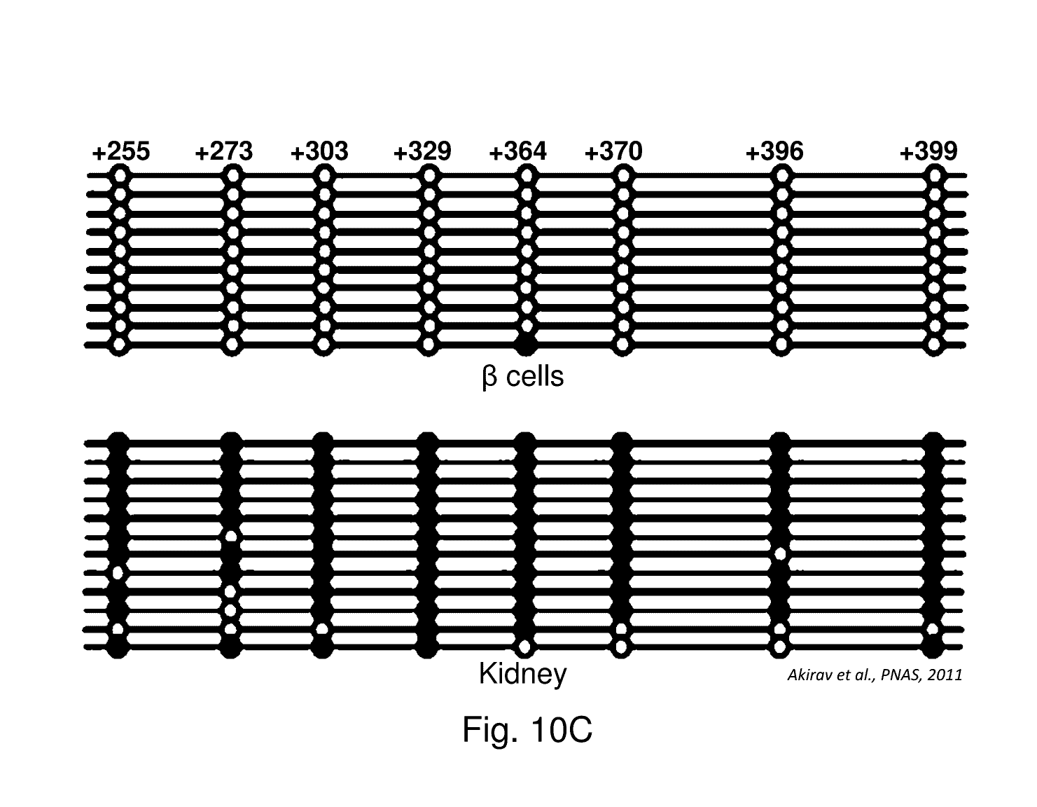

FIG. 10A shows that methylation patterns are highly conserved in human and mouse insulin. FIGS. 10B and 10C show that human kidney and human islet beta cells show differential methylation patterns.

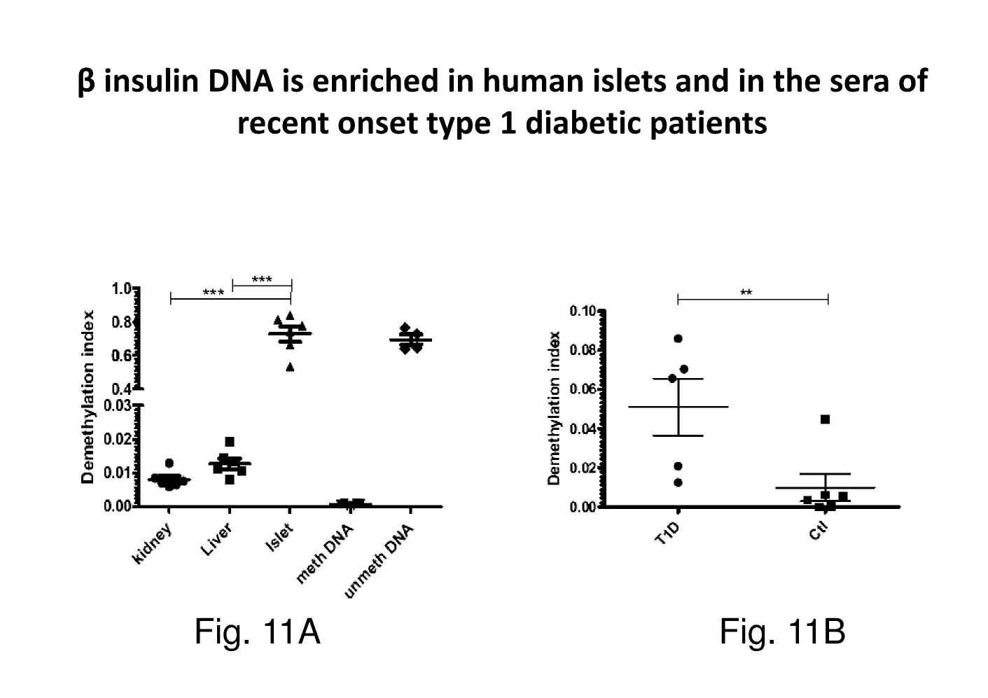

FIG. 11A shows that the demethylation index of insulin DNA from islet cells is high, and corresponds to authentic unmethylated insulin DNA. FIG. 11B shows that insulin DNA is enriched in human islets and in the sera of recent onset type 1 diabetic patients vs. controls.

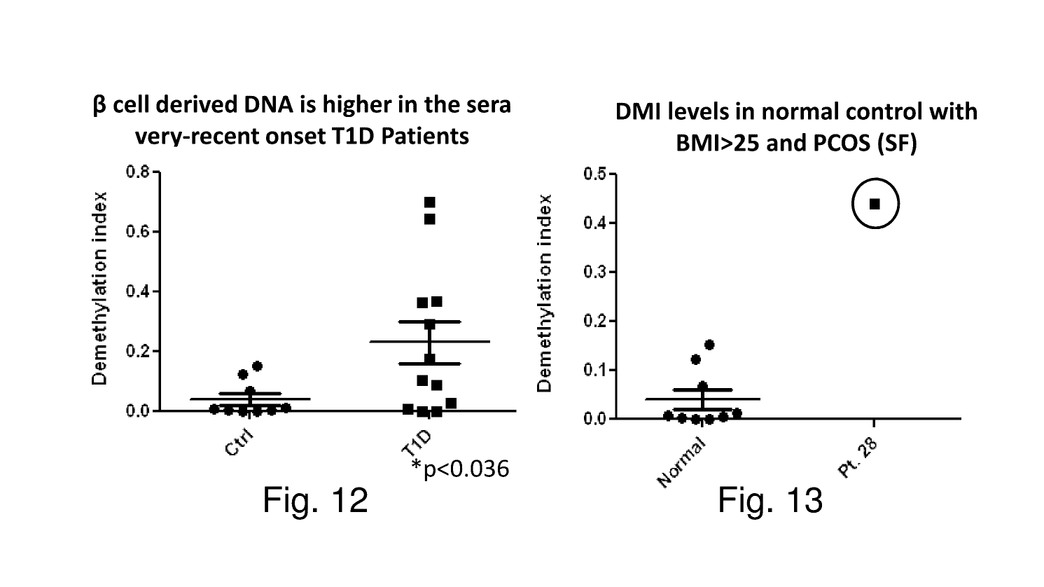

FIG. 12 shows a graph demonstrating that β cell derived DNA is significantly higher in the sera very-recent onset T1D Patients.

FIG. 13 shows a graph of DMI levels in normal controls with BMI>25 and a patient, demonstrating use of the technology as a diagnostic or prognostic tool.

A pilot study was designed to investigate very-recent onset type 1 diabetes (T1D) patients, as follows:

Study design:

Human Subjects—

Sample size: 15×T1D and 15× age/sex/race matched controls.

10-19 year old T1D patients are enrolled within 90 days of diagnosis.

Selection Criteria—

10 to 19 years old male and female subjects with and without type 1 diabetes.

Type 1 diabetes will be defined as random glucose levels higher than >200 mg/dL.

AutoAb levels determination

Exclusion Criteria—

Chronic treatment; other autoimmune conditions, such as rheumatoid arthritis, multiple sclerosis, or Hashimoto's thyroiditis; type 2 diabetes, secondary diabetes or Maturity onset diabetes of youth.

Primary Analysis—

Examination of β cell derived DNA levels in T1D and HC subjects.

Secondary Analysis—

Mix meal tolerance test (MMTT). C-peptide levels and glucose responses measured 0, 15, 30, 60, 90, and 120 minutes after ingestion.

HA1c (at diagnosis and at sampling)

AutoAb measures

Sample type—includes serum and plasma to evaluate assay performance.

Table 1 shows Subject/control characteristics of the T1D Biomarker study.

TABLE 1

Group

| | Ctrl | T1D |
|---|---|---|
| N | 15 | 15 |
| Age | 13.73 ± 0.58 | 13.53 ± 0.65 |
| F/(M) | 10/(5) | 10/(5) |
| HA1c (Dx) | | 12.17 ± 0.78% |
| Duration of T1D (wks) | — | 5.7 ± 0.80 |
| HA1c (Visit) | −5.26 ± 0.07% | 9.39 ± 0.59% |
| # AutoAb | 0 | 2.26 ± 0.18 |
| DKA (Dx) Y/(N) | — | 9/(9) |

### Example 2

### Oligodendrocyte Loss in Multiple Sclerosis

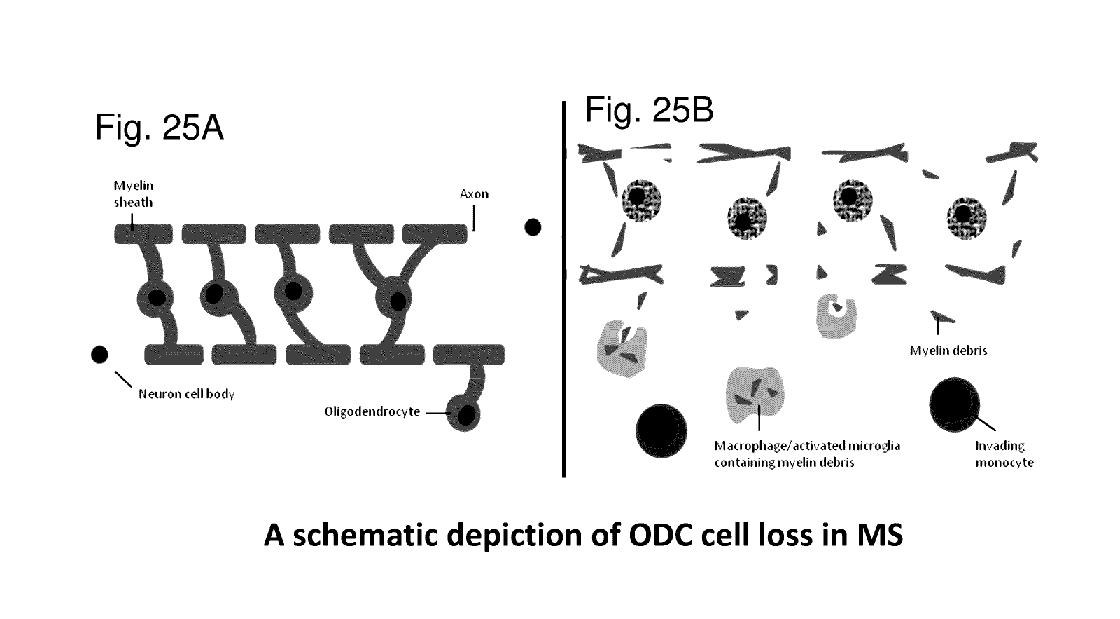

DNA methylation is a basic mechanism by which cells regulate gene expression, and while all cells share an identical DNA sequence, DNA methylation varies considerably according to cell function. Earlier studies by the inventor developed a minimally invasive method for detecting beta cell loss in the autoimmune disease, type 1 diabetes (Akirav et al. PNAS 2011). This assay detects differentially methylated DNA that is released from dying beta cells into the blood of patients with diabetes. Similarly, oligodendrocyte (ODC) DNA is released upon cell loss, and that this DNA can be detected in the blood of patients. The ability to detect ODC loss provides a biomarker for MS development, progression, and clinical response to therapy. FIGS. 25A and 25B show a schematic depiction of ODC cell loss in MS, with healthy tissue shown in FIG. 25A, and pathological tissue in FIG. 25B.

ODCs serve as a primary target of the immune system in the CNS. Myelin oligodendrocyte glycoprotein (MOG), a key component of the myelin sheath, is produced by ODCs and has long been studied as a primary antigen in MS. Preliminary data show low MOG-DNA methylation levels in human and mouse brains and purified ODC. These differences in DNA sequence can be detected by methylation sensitive primers, thereby determining the origin of the DNA. The following experimental approach may be used to determine whether ODC DNA levels increase in mouse and human MS:

1) Examining ODC MOG-DNA in the blood of mice with MOG-induced experimental autoimmune encephalomyelitis (EAE). EAE is often used to induce ODC loss and paralysis in mice. The assay may be used to detect ODC loss prior to and following disease presentation by analyzing blood from MOG35-55 immunized mice. Levels of ODC MOG DNA are tested against clinical and histological markers in the CNS.

2) Examining ODC MOG-DNA In the blood of MS patients—The levels of MOG-DNA in the blood of MS patients are examined. Patient and control samples are obtained from both commercial sources, as well as, a clinical MS patient population.

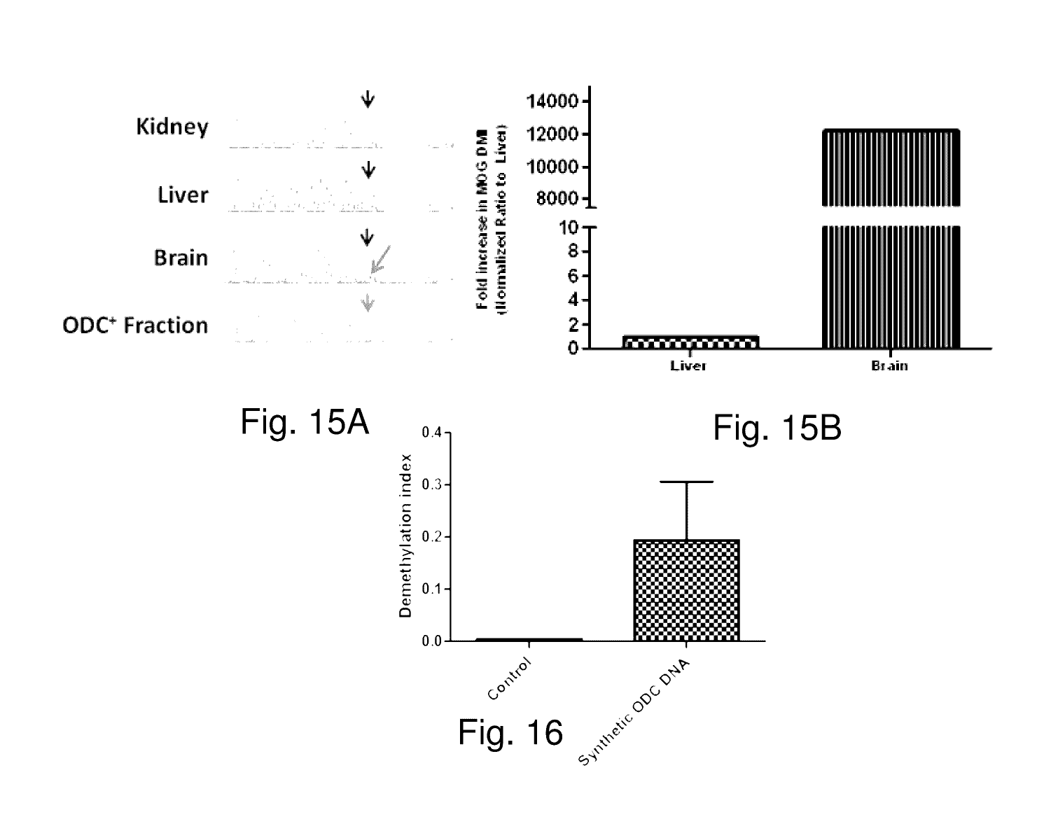

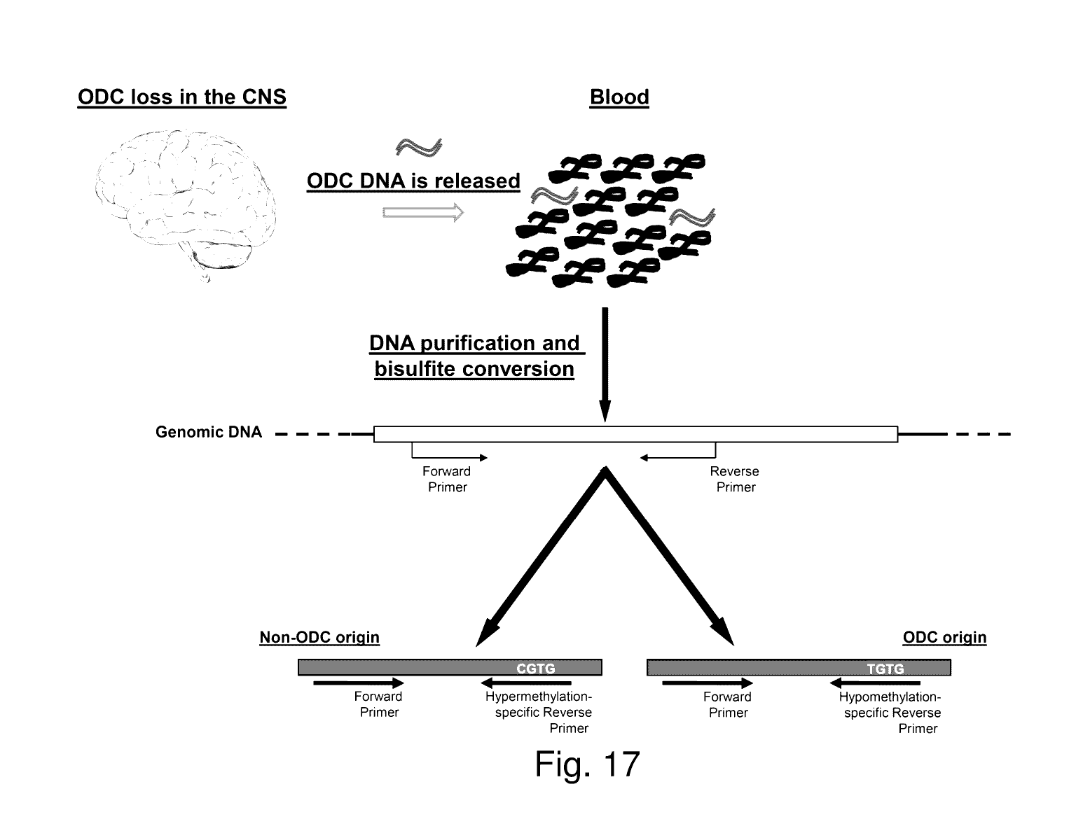

Preliminary data show that MOG-DNA methylation is reduced in mouse brain and purified ODCs, while liver and kidney show hypermethylation of the DNA (FIG. 15B). Similar results were also obtained when human liver and brain tissues were compared; demonstrating that demethylation of the MOG gene was in the brain (data not shown).

Primary tissues of mice were collected to determine the methylation state of the MOG gene. An enriched fraction of ODCs was isolated by magnetic sorting. For EAE induction, mice received one injection of MOG in CFA and PT toxin. EAE was monitored daily and blood collected every 7 days.

Serum samples from healthy human controls and patients with untreated active RRMS were purchased from Applied Biological Services (ABS).

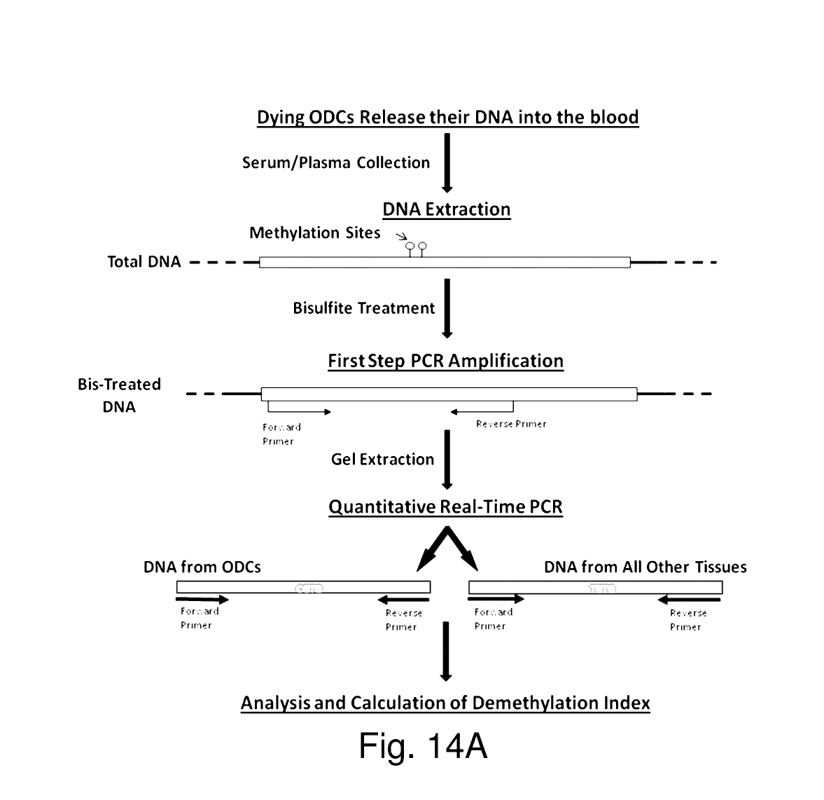

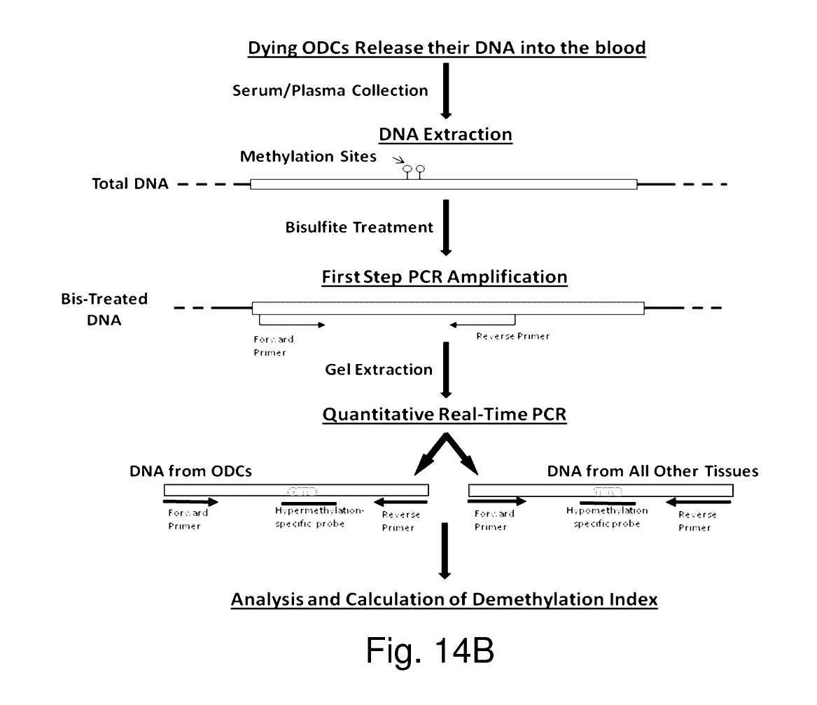

The assay is based on the bisulfite DNA conversion reaction. During this reaction, methylated cytosines (C) are protected from bisulfite conversion while demethylated C's are converted to uracils. Therefore, this reaction generates a distinct DNA sequence based on the methylation state of the DNA (FIGS. 14B and 14C).

These changes in DNA sequence can be detected using methylation sensitive primers that are specific for hypermethylated and demethylated MOG-DNA. In the context of MS, demethylated MOG-DNA can serve as a biomarker of ODCs DNA, as it is released into circulation by dying ODCs. A ratio representing the relative abundance of demethylated DNA is described as the demethylation index (DMI) (Akirav et. al. (12)) shown in FIG. 14B. Alternately, DNA probes which are specific for methylated and demethylated MOG gene may be used to quantitatively determine the DMI, as shown in FIG. 14C.

A test was conducted to determine whether demethylated sensitive primers can detect ODC DNA in brain samples. DNA was purified from murine liver, kidney and brain tissues. In addition purified ODCs from mouse brains were enriched using magnetic bead directed against the cell surface marker, 04, and DNA was collected. FIG. 15A shows differences in Sanger sequencing based on tissue of origin. FIG. 15B shows a 10 fold increase in demethylated MOG-DNA in the brain when compared with liver and kidney tissues. MOG DMI values were further increased when purified O4+ ODCs were examined (>250 folds).

Similar results were also observed using primary human tissues, with human brain DNA showing a 12,000 fold increase in demethylated MOG-DNA levels compared with liver (FIG. 22).

The addition of ODCs DNA to mouse serum (spiking) showed the ability of the assay to detect DNA amounts equal to those of five ODCs in 300 μL of serum (data not shown).

Next, the ability of the assay to detect synthetic ODC DNA fragments was determined. Mice receiving plasmids containing ODC DNA fragments were euthanized and DNA extracted from the serum. Methylation sensitive primers showed elevated levels of MOG DNA when compared with untreated control mice. FIG. 16 shows that blood from mice receiving artificially demethylated MOG DNA show a high deMeth MOG signature. Mice were injected with synthetic MOG DNA and serum collected 2-4 minutes post injection. Data shows the ability of the assay to detect ODC MOG-DNA in the blood of mice which received synthetic DNA injection. There were 3 mice per group, and a statistical analysis shows that p<0.06.

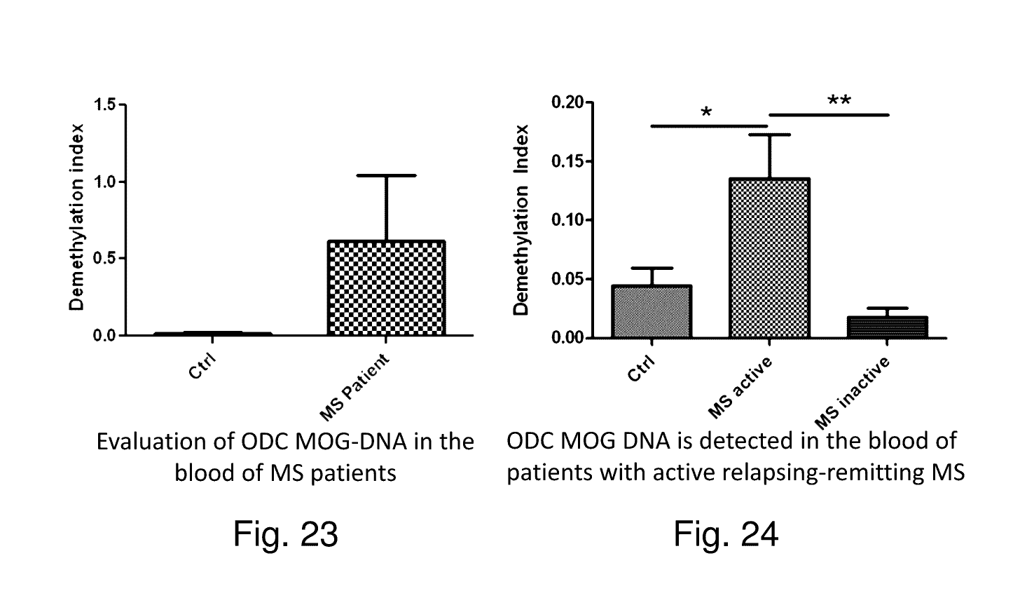

Finally, the levels of ODC MOG-DNA in 5 human patients with early MS and 5 controls was examined. Methylated sensitive PCR analysis showed increased levels of ODC DNA in 3 out of the 5 patients with MS, demonstrating the utility of this assay in detect active ODC loss in the brain (FIG. 23).

Bisulfite Treatment

DNA from serum samples can be purified using the Qiagen QIAamp DNA Blood Kit following the manufacturer-recommended protocol. The DNA can then be subjected to bisulfite treatment and purified on a DNA binding column to remove excessive bisulfite reagent using the Zymo EZ DNA Methylation Kit.

First-Step PCR and Gel Extraction.

A methylation-independent reaction can be carried out to increase the DNA template for PCR analysis.

For the reaction, bisulfite-treated DNA template can be added to Zymo Taq Premix. The amplification is conducted for, e.g., 50 cycles. The PCR products can be excised from a 3% agarose gel. Negative controls without DNA should not yield products in the first-step reaction.

PCR products obtained using methylation-independent primers can be purified using a Qiagen PCR Purification Kit.

The following primers and probes are available to implement an embodiment of the method:

Human MOG
Probe Set 1:
Hypermethylated strand (non oligodendrocyte)-
SEQ ID NO: 013
Forward primer- GGGTAGTTTAGAGTGATAGGATTAAGATAT
SEQ ID NO: 014
Reverse Primer- TAAAAATAAACCACCCTAAAAAAAA
SEQ ID NO: 015
Probe- AACGTTCTTCCCAAAAAATATACGA
Hypomethylated strand (non oligodendrocyte)-
SEQ ID NO: 016
Forward primer- GGGTAGTTTAGAGTGATAGGATTAAGATAT
SEQ ID NO: 017
Reverse Primer- TAAAAATAAACCACCCTAAAAAAAA
SEQ ID NO: 018
Probe- AACATTCTTCCCAAAAAATATACAA
Probe Set 2:
Hypermethylated strand (non oligodendrocyte)-
SEQ ID NO: 019
Forward primer- GGGTAGTTTAGAGTGATAGGATTAAGATAT
SEQ ID NO: 020
Reverse Primer- TAAAAATAAACCACCCTAAAAAAAA
SEQ ID NO: 021
Probe- TACGACATAACAATTCCACTTCATCCCCGA
Hypomethylated strand (non oligodendrocyte)-
SEQ ID NO: 022
Forward primer- GGGTAGTTTAGAGTGATAGGATTAAGATAT
SEQ ID NO: 023
Reverse Primer- TAAAAATAAACCACCCTAAAAAAAA
SEQ ID NO: 024
Probe- TACAACATAACAATTCCACTTCATCCCCAA
Mouse MOG
Probe Set 1:
Hypermethylated strand (non oligodendrocyte)-
SEQ ID NO: 025
Forward primer- GAGTGATAGGATTAGGGTATTTTATT
SEQ ID NO: 026
Reverse Primer- TCTACATCTTAATCCTTACCATTTC
SEQ ID NO: 027
Probe- ACCCGTAACATTTTTCCCAAAAAAAATACGA
Hypomethylated strand (non oligodendrocyte)-
SEQ ID NO: 028
Forward primer- GAGTGATAGGATTAGGGTATTTTATT
SEQ ID NO: 029
Reverse Primer- TCTACATCTTAATCCTTACCATTTC
SEQ ID NO: 030
Probe- ACCCATAACATTTTTCCCAAAAAAAATACAA
Probe Set 2:
Hypermethylated strand (non oligodendrocyte)-
SEQ ID NO: 031
Forward primer- GAGTGATAGGATTAGGGTATTTTATT
SEQ ID NO: 032
Reverse Primer- TCTACATCTTAATCCTTACCATTTC
SEQ ID NO: 033
Probe- TACGACACGACAACTCTACTTCAT
Hypomethylated strand (non oligodendrocyte)-
SEQ ID NO: 034
Forward primer- GAGTGATAGGATTAGGGTATTTTATT
SEQ ID NO: 035
Reverse Primer- TCTACATCTTAATCCTTACCATTTC
SEQ ID NO: 036
Probe- TACAACACAACAACTCTACTTCAT

Methylation-Specific Analysis

Methylation-specific DNA probes can be used for the detection of oligodendrocyte derived DNA. These probes are able to quantitatively and sensitively detect circulation demethylated and methylated DNA from oligodendrocyte and non-oligodendrocyte origin, respectively. Alternately, methylation specific primers may be employed (see, e.g., Akirav E M, Lebastchi J, Galvan E M, Henegariu O, Akirav M, Ablamunits V, Lizardi P M, and Herold K C. Detection of beta cell death in diabetes using differentially methylated circulating DNA. PNAS, 2011, Proceedings of the National Academy of Sciences, 2011, November:108(19018-23), expressly incorporated herein by reference, hereinafter Akirav et al. (2011). See also Husseiny M. I., Kuroda A., Kaye A. N., Nair I., Kandeel F., et al. (2012) Development of a Quantitative Methylation-Specific Polymerase Chain Reaction Method for Monitoring Beta Cell Death in Type 1 Diabetes. PLoS ONE 7(10): e47942. doi:10.1371/journal.pone.0047942, expressly incorporated herein by reference), which presented with a relatively low specificity (i.e. demethylated primers detected methylated DNA and vice versa). Low specificity negatively impacts assay sensitivity by decrease detection limits of demethylated DNA. Low DNA levels are presumably present during early cell loss. See, U.S. Pat. No. 6,150,097, expressly incorporated herein by reference.

The overall procedure for the detection of circulating oligodendrocyte DNA is depicted in FIGS. 1, 14B and 14C. The steps leading to the use of probes are similar to those described in Akirav et al. (2011), which discloses the use of methylation-specific primers (and not probes) to detect beta cell derived DNA. The primers were able to detect demethylated and methylated DNA from a beta cell and a non-beta cell origin, respectively. While useful, these primers had a relatively low specificity whereby demethylated primers detected methylated DNA and vice versa. Low specificity reduced assay sensitivity as it impaired the ability to detect very low levels of beta cell-derived DNA, such as in the condition of early beta cell loss and pre-diabetes.

DNA from serum samples may be purified using the Qiagen QIAamp DNA Blood Kit following the manufacturer-recommended protocol. Synthetic unmethylated and methylated DNA is available from Millipore. Purified DNA may be quantitated using a NanoDrop 2000 spectrophotometer. DNA may then be subjected to bisulfite treatment and purified on a DNA binding column to remove excessive bisulfite reagent using the Zymo EZ DNA Methylation Kit.

Probe DNA may offer a significant improvement in sensitivity over primers used for quantitative PCR. That is, probe DNA allows for a highly specific recognition of demethylated MOG gene DNA. This would tend to eliminate false positive readings and thus provide increased assay specificity and sensitivity.

A methylation-independent reaction may be carried out to increase the DNA template for PCR analysis. For the reaction, bisulfite-treated DNA template was added to ZymoTaq™ Premix (see, www.zymoresearch.com/protein/enzymes/zymotaq-dna-polymerase, expressly incorporated herein by reference.) Using the forward and reverse primers, PCR may be conducted, and the PCR products excised from a 3% agarose gel.

The PCR product (or amplicon) may be detect by methylation status specific probes as follows:

a) Probes for the detection of methylated MOG DNA (i.e., DNA not derived from a oligodendrocyte):

SEQ ID NO: 015
Human Probe 1- AACGTTCTTCCCAAAAAATATACGA
SEQ ID NO: 021
Human Probe 2- TACGACATAACAATTCCACTTCATCCCCGA
SEQ ID NO: 027
Mouse Probe 1- ACCCGTAACATTTTTCCCAAAAAAAATACGA
SEQ ID NO: 033
Mouse Probe 2- TACGACACGACAACTCTACTTCAT

b) Probes for the detection of demethylated MOG DNA (i.e., DNA derived from an oligodendrocyte):

SEQ ID NO: 018
Human Probe 1- AACATTCTTCCCAAAAAATATACAA
SEQ ID NO: 024
Human Probe 2- TACAACATAACAATTCCACTTCATCCCCAA
SEQ ID NO: 030
Mouse Probe 1- ACCCATAACATTTTTCCCAAAAAAAATACAA
SEQ ID NO: 033
Mouse Probe2- TACAACACAACAACTCTACTTCAT

c) PCR is done with an annealing temperature of 60° C. for 50 cycles and quantified using a Real Time PCR machine. A range of 52-65° C. for the PCR would be acceptable.

d) Values generated by demethylated probes are subtracted from values of methylated probes and a delta (difference) calculated.

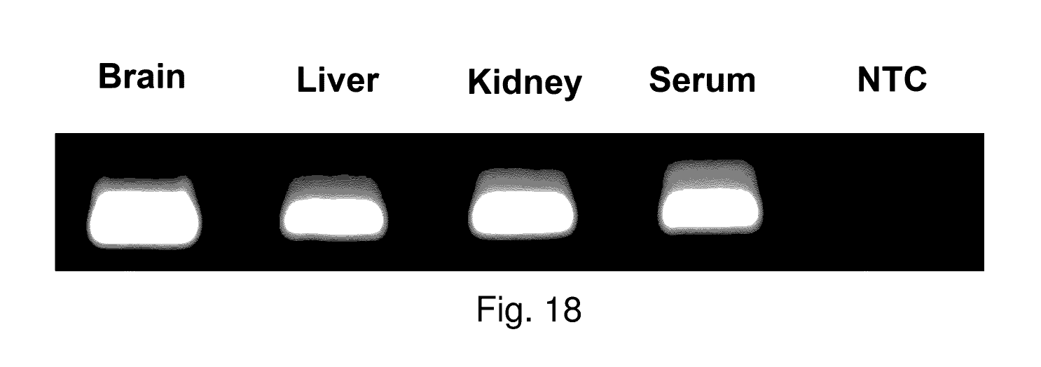

FIG. 18 shows the extent to which the first step of PCR in mouse samples detects MOG DNA in different mouse tissues.

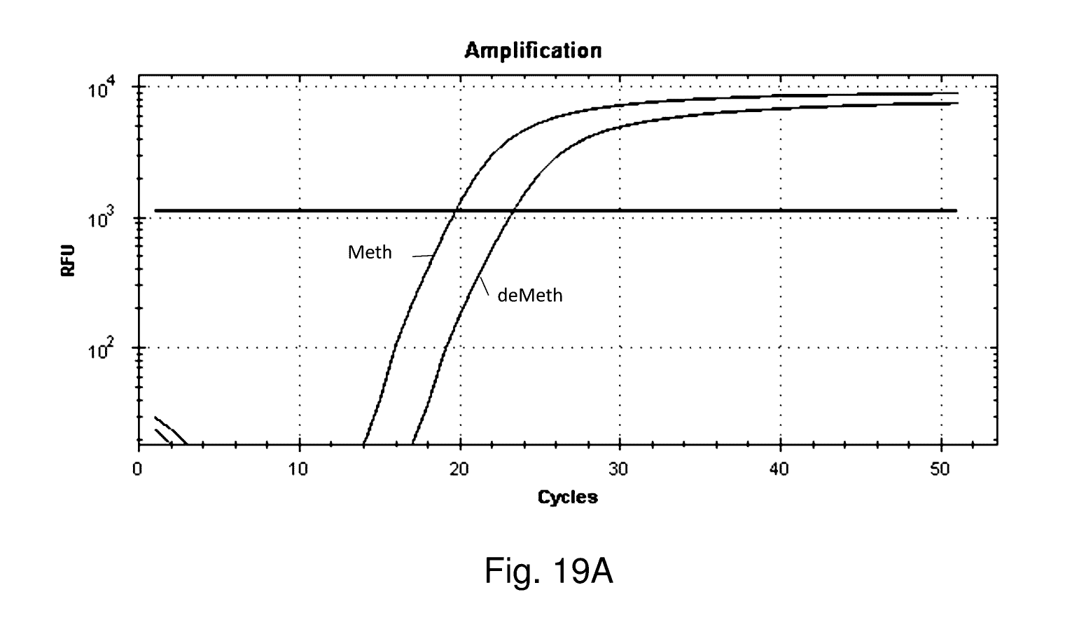

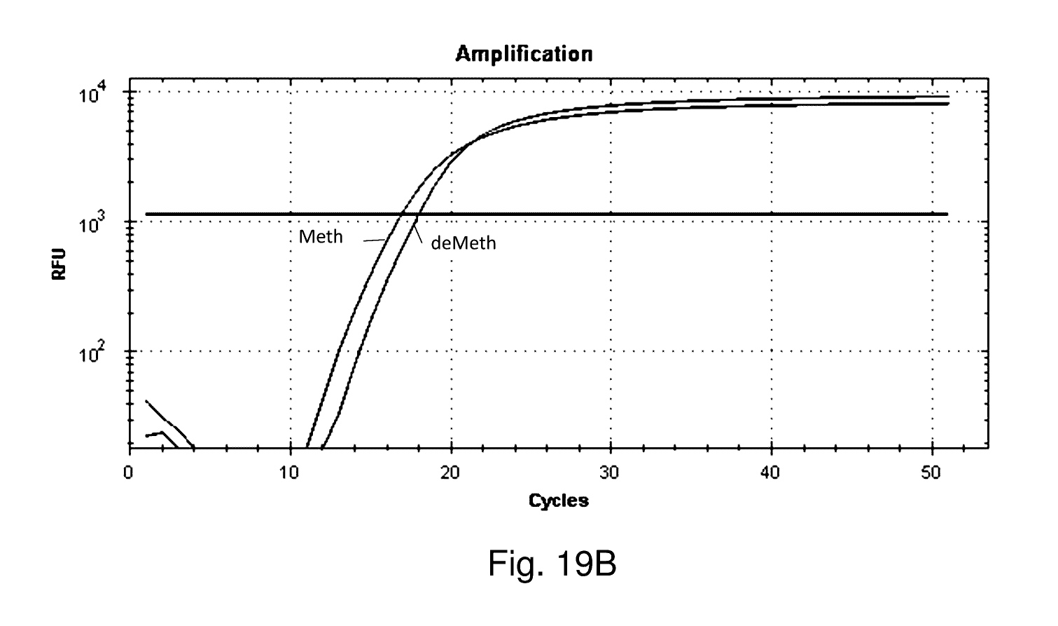

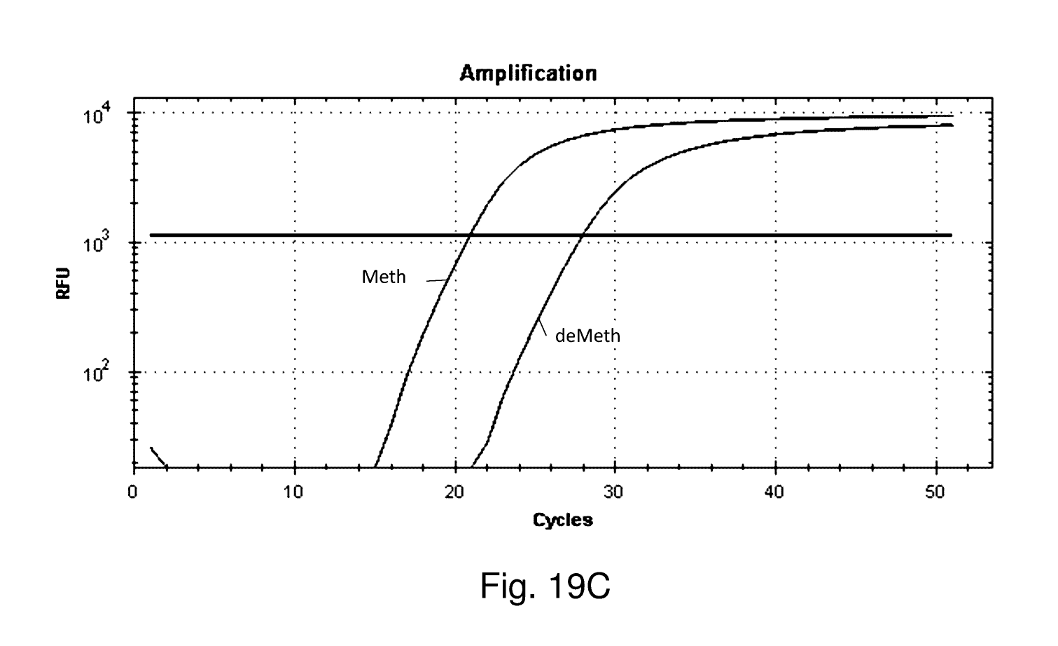

FIGS. 19A-19C show graphs or real time PCT results of different murine tissues, which demonstrate that Methylation specific primers detect high levels of deMeth MOG DNA in the CNS of mice (FIGS. 19A and 19B), but not in liver (FIG. 19C). The O4⁺ ODC fraction was obtained by magnetic bead isolation.

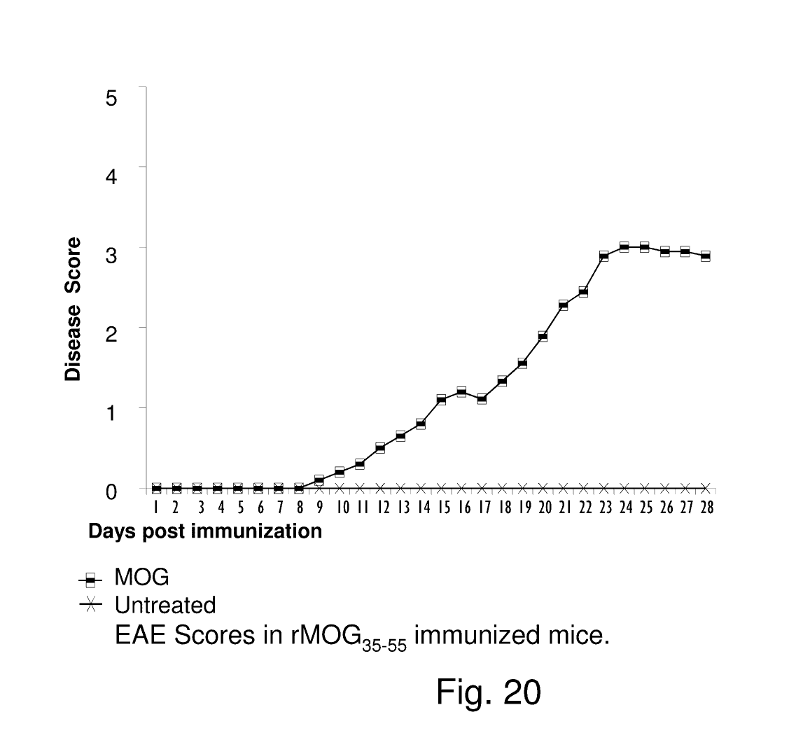

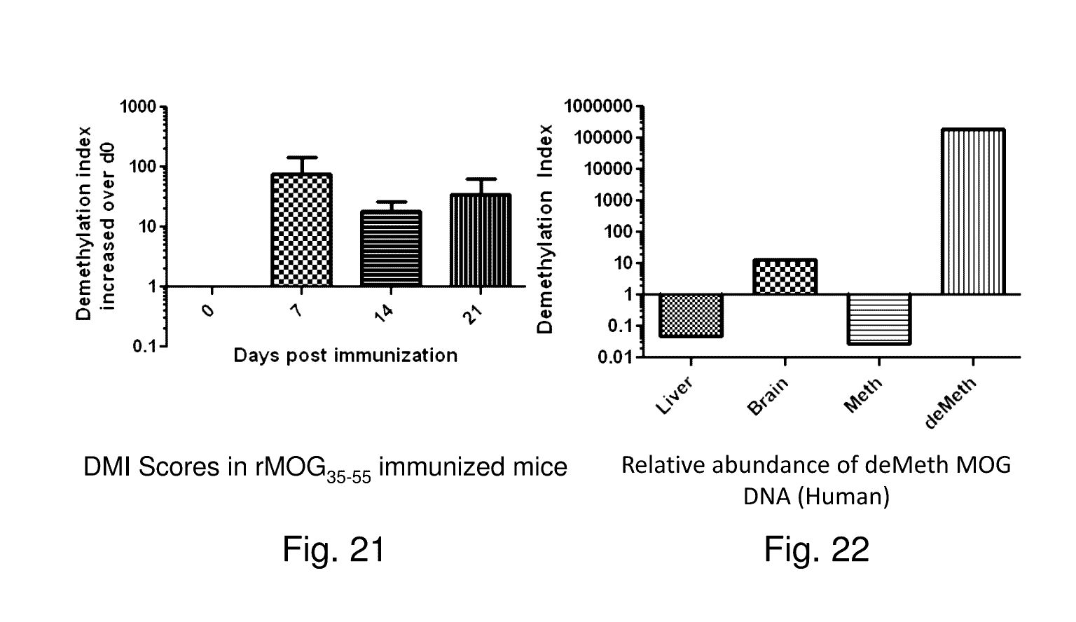

FIG. 20 shows EAE scores in rMOG35-55 immunized mice as a function of days post immunization. FIG. 21 shows DMI scores in rMOG35-55 immunized mice.

FIG. 22 shows a relative abundance of deMeth MOG DNA in human tissues.

FIG. 23 shows that human patients with RMMS show higher levels of ODC MOG DNA than human controls.

FIG. 24 shows a bar graph showing that ODC MOG DNA is detected in the blood of patients with active relapsing-remitting MS.

In various embodiments, the methylation status-specific probes are conjugated with 6-carboxyfluorescein, abbreviated as FAM, thus permitting quantitative detection. See, en.wikipedia.org/wiki/TaqMan, expressly incorporated herein by reference.

### Example 3

### Murine MOG

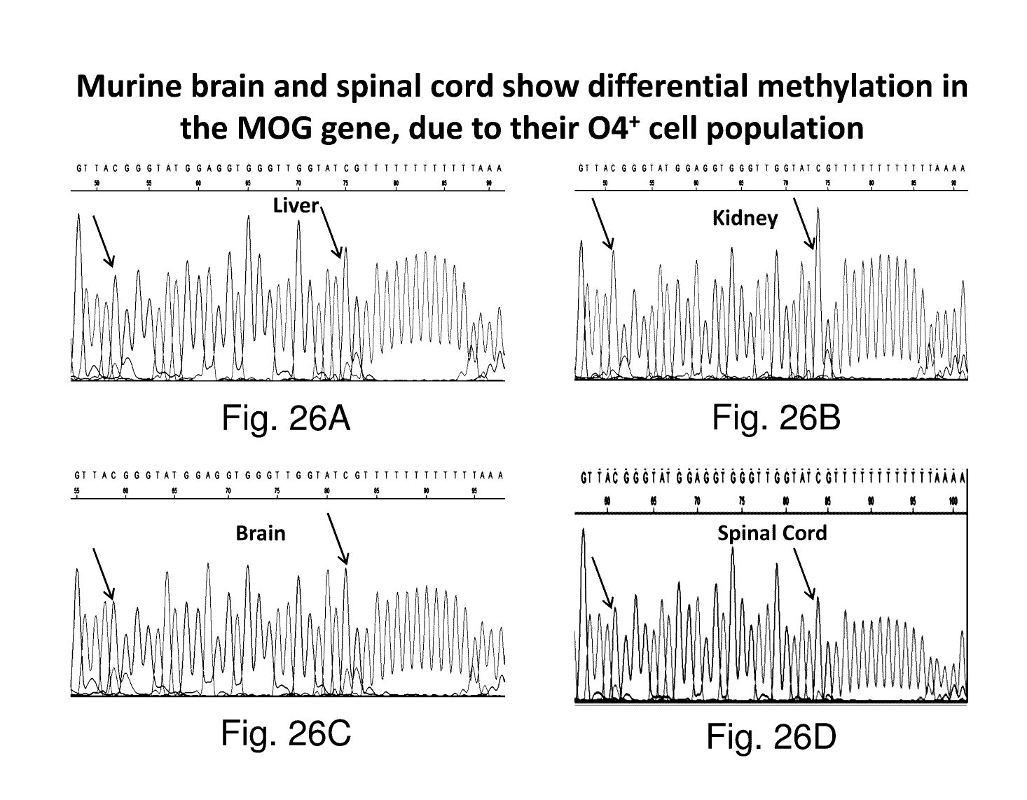

Murine brain and spinal cord show differential methylation in the MOG gene, due to their O4⁺ cell population. FIGS. 26A-26C show Sanger sequencing results of bisulfite treated DNA from murine tissues. The arrows point toward CpG sites where cytosines (C) are preserved in methylated samples (Liver, Kidney), or converted to thymines (T) in samples containing demethylated CpGs, leading to a mixed population of C's and T's (Brain, Spinal Cord).

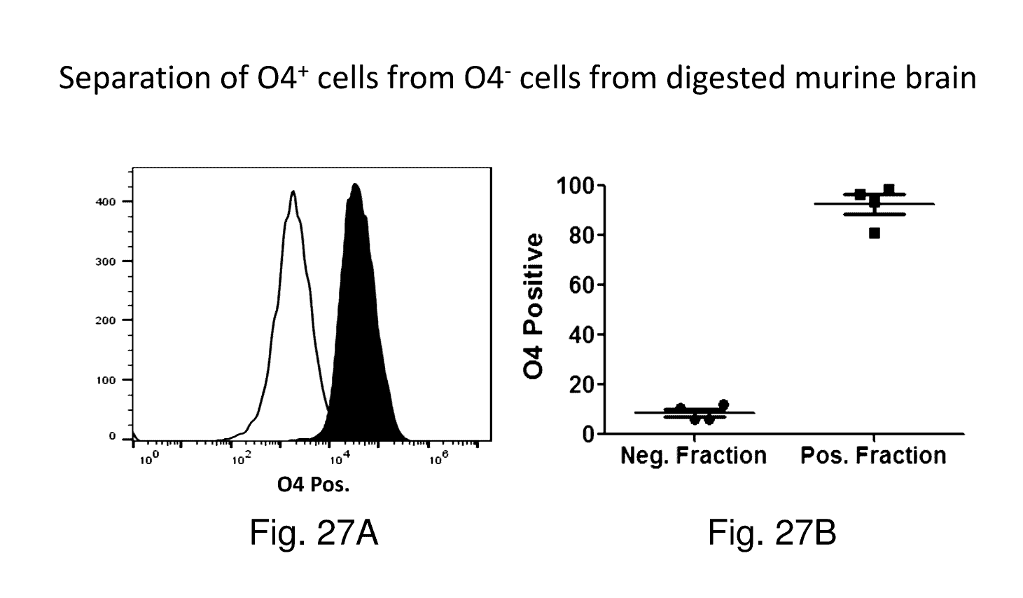

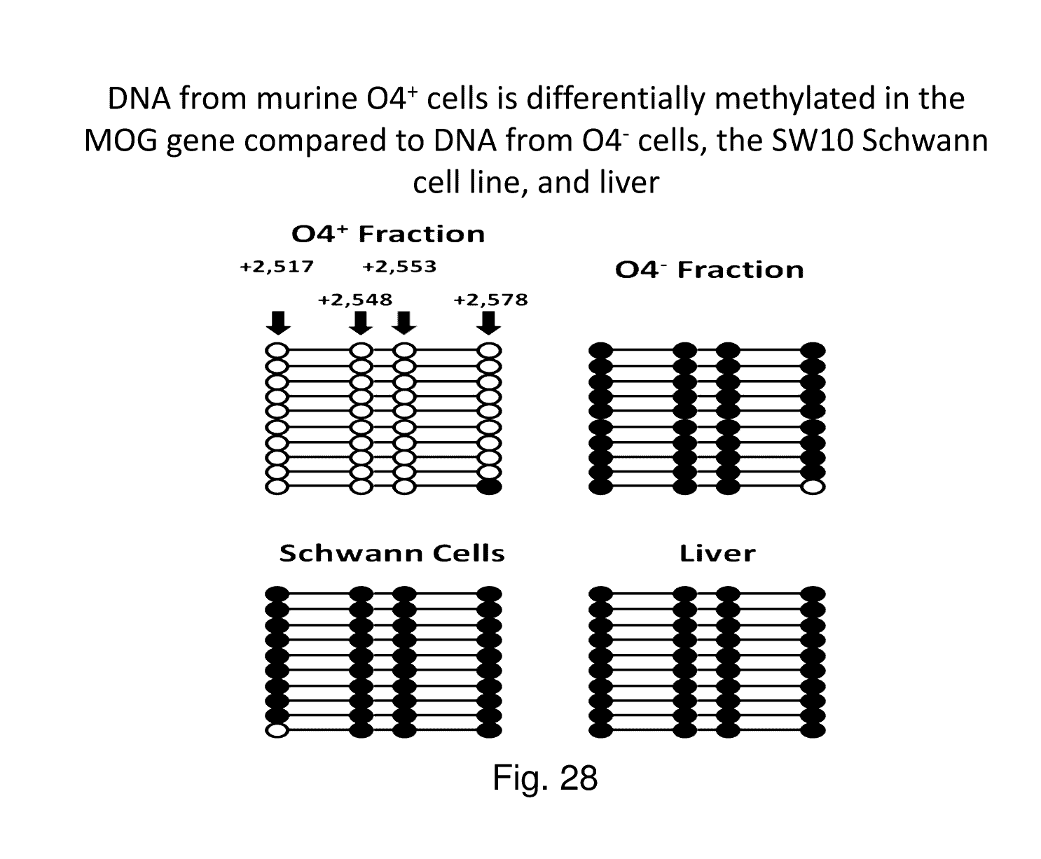

FIGS. 27A and 27B show separation of O4⁺ and O4⁻ cells from digested murine brain tissue by magnetic beads. O4⁺ and O4⁻ cells were separated from digested murine brain tissue by magnetic beads. FACS analysis showed >92.6±3.9% enrichment of O4⁺ cells among four independent preparations when compared to O4⁻ fractions. FIG. 28 shows DNA from murine O4⁺ cells is differentially methylated in the MOG gene compared to DNA from O4⁻ cells, the SW10 Schwann cell line, and liver. Sequence analysis was performed on first-step PCR product of each sample, 10 clones from each are shown (◯ represent demethylated cytosines; •, methylated cytosines). Locations in relation to the MOG transcription start site are listed, methylation-specific murine primers incorporate the CpG site at by +2,553.

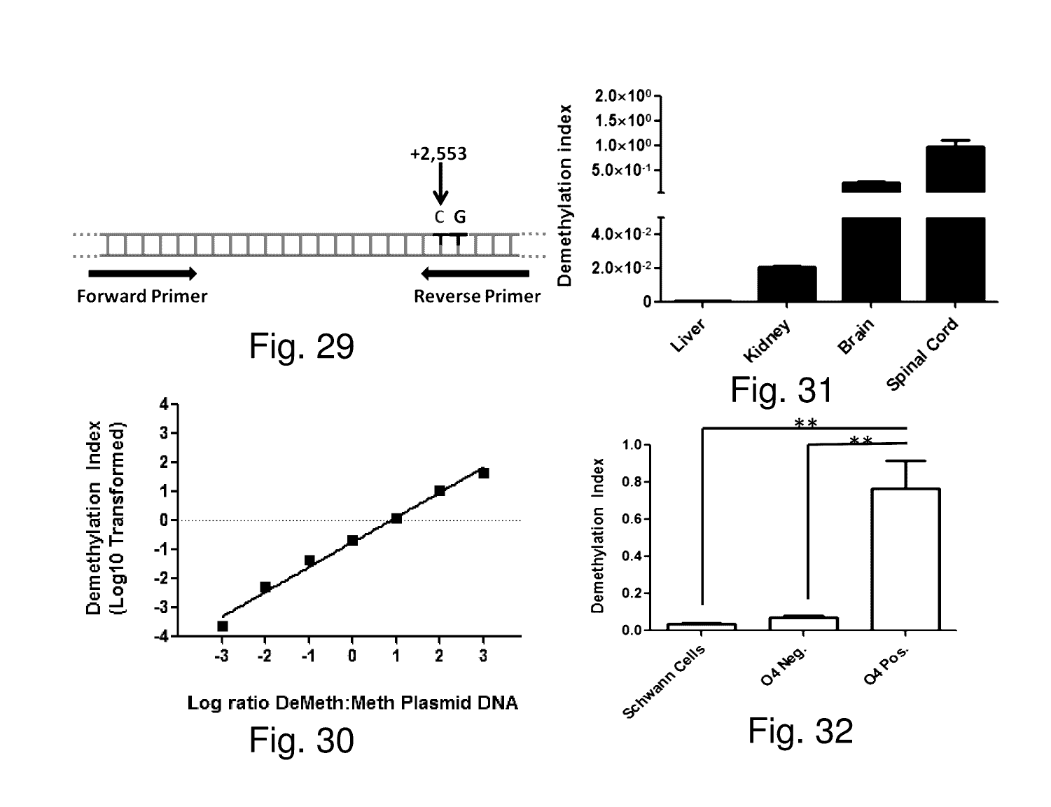

Methylation-specific primers display high specificity and sensitivity and can detect demethylated MOG DNA in murine brain, spinal cord, and O4⁺ cells. FIG. 29 shows a depiction of MOG gene region utilized for mouse methylation-specific qPCR analysis; the cytosine at by +2,553 from MOG transcription start site incorporated into reverse primer sequence. FIG. 30 shows the demethylation index determined using methylation-specific primers tested using plasmids containing methylated and demethylated murine MOG DNA inserts over a wide range of serial dilutions (R²=0.987, p<0.0001). FIG. 31 shows that methylation-specific primers were used in qPCR with bislufite treated DNA from murine liver, kidney, brain, and spinal cord. Three independent analyses used to compute DMI averages; Liver vs. Brain/Spinal Cord p<0.001, Kidney vs. Brain/Spinal Cord p<0.001. FIG. 32 shows the demethylation index determined using methylation-specific primers in qPCR with murine O4⁺ cells, O4⁻ cells, and SW10 Schwann cells. Three independent analyses used to compute DMI averages; ANOVA p=0.0008, O4⁺ vs. O4⁻ p<0.01, O4⁺ vs. SW10 Schwann cells p<0.01.

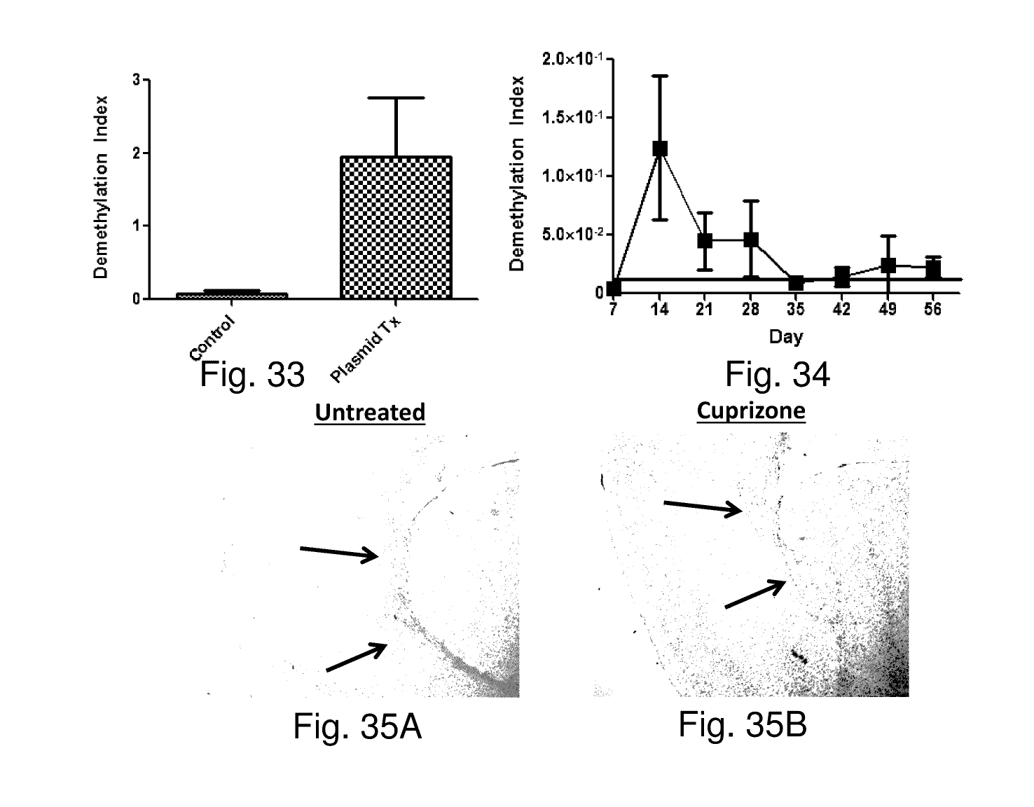

Methylation-specific primers can detect DeMeth MOG DNA in serum of plasmid injected mice and in cuprizone treated mice which can be correlated with demyelination in neural tissue. FIG. 33 shows a bar graph comparing results of methylation-specific primers to detect DeMeth MOG DNA in bisulfite-treated DNA from serum of mice injected with DeMeth MOG plasmid and cuprizone-treated non-injected mice, run on qPCR with methylation-specific primers; Tx vs. Ctrl p<0.012. FIG. 34 shows a graph of bisulfite-treated DNA from serum of cuprizone-fed mice (n=12), run on qPCR with methylation-specific primers; MOG DMIs peak at Day 14 and remain elevated over baseline until Day 35. FIGS. 35A and 35B show brain sections from cuprizone-fed and control mice stained for myelin using Luxol fast-blue; arrows indicate region of native myelination.

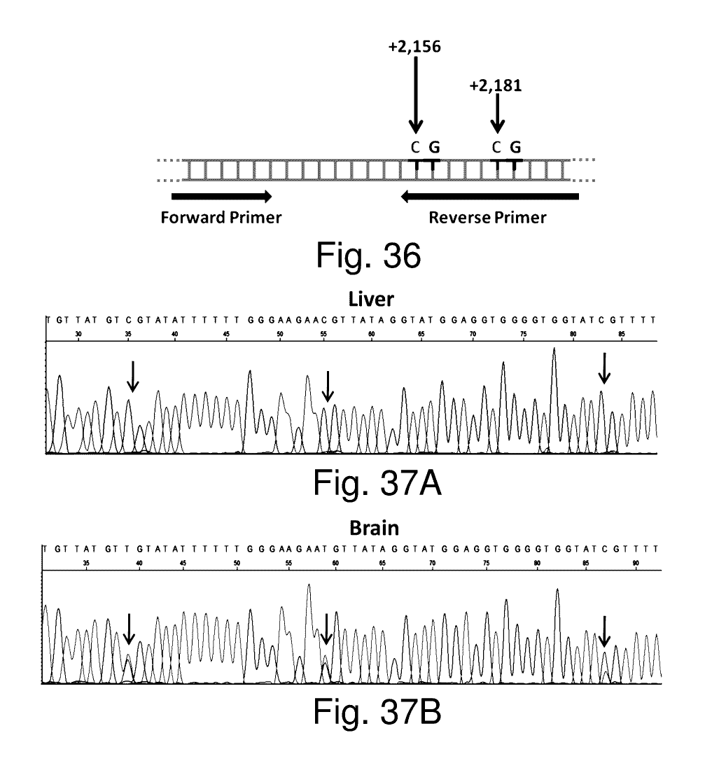

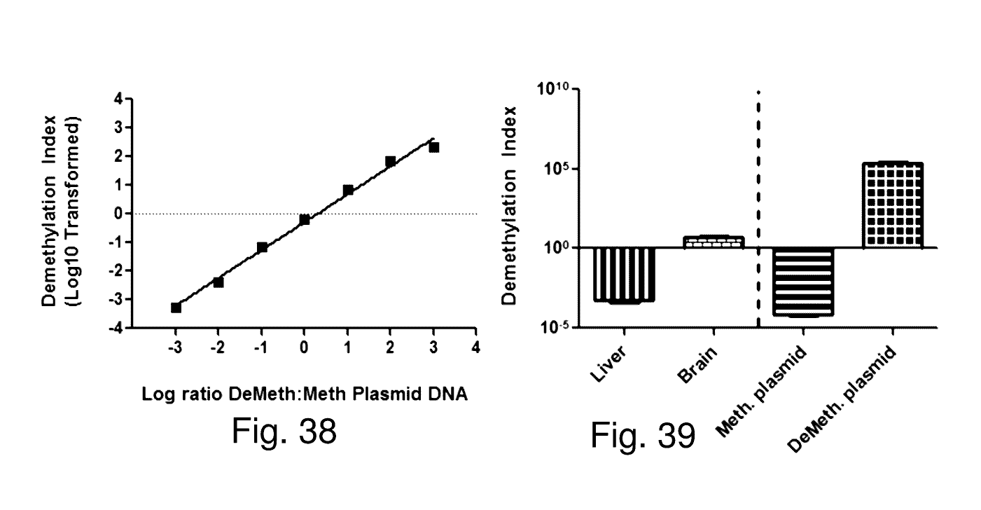

Methylation-specific primers display high specificity and sensitivity and can detect demethylated MOG DNA in human brain and liver. FIG. 36 depicts the MOG gene region utilized for human methylation-specific qPCR analysis; cytosines at bps +2,156 and +2,181 from MOG transcription start site incorporated into reverse primer sequence. FIGS. 37A and 37B show Sanger sequencing results of bisulfite treated DNA from liver and brain human tissues, respectively, in which the -most arrows point toward CpG sites where cytosines (C) are preserved in methylated sample (Liver), or converted to thymines (T) in sample containing demethylated CpGs, leading to a mixed population of C's and T's (Brain); red arrows (left and middle) indicate CpGs incorporated into reverse primers. FIG. 38 shows a logarithmic graph of methylation- specific primers tested using plasmids containing methylated and demethylated human MOG DNA inserts over a wide range of serial dilutions (R²=0.992, p<0.0001). FIG. 39 shows a bar graph of methylation-specific primers used in qPCR with bisulfite treated DNA from human liver, brain, and Meth and Demeth MOG plasmids. Three independent analyses used to compute DMI averages; Liver vs. Brain p<0.003.

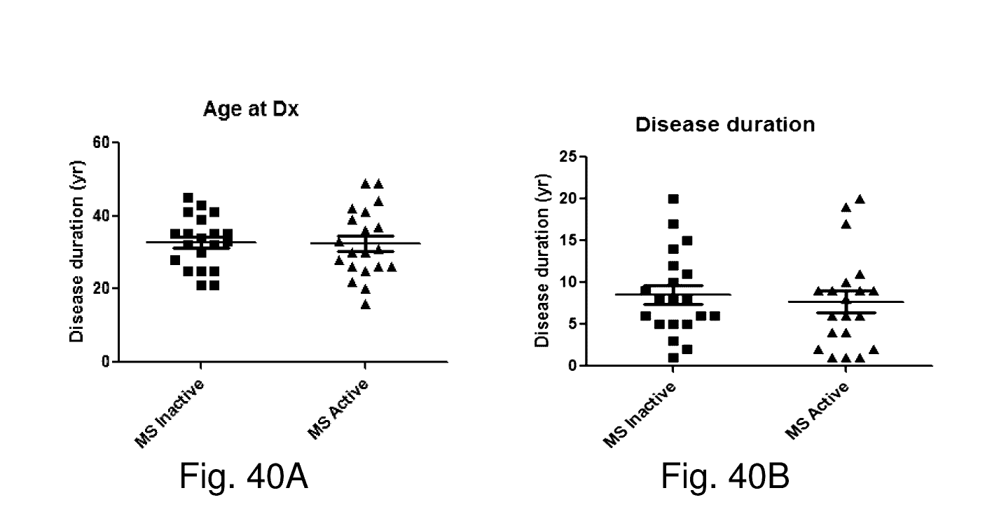

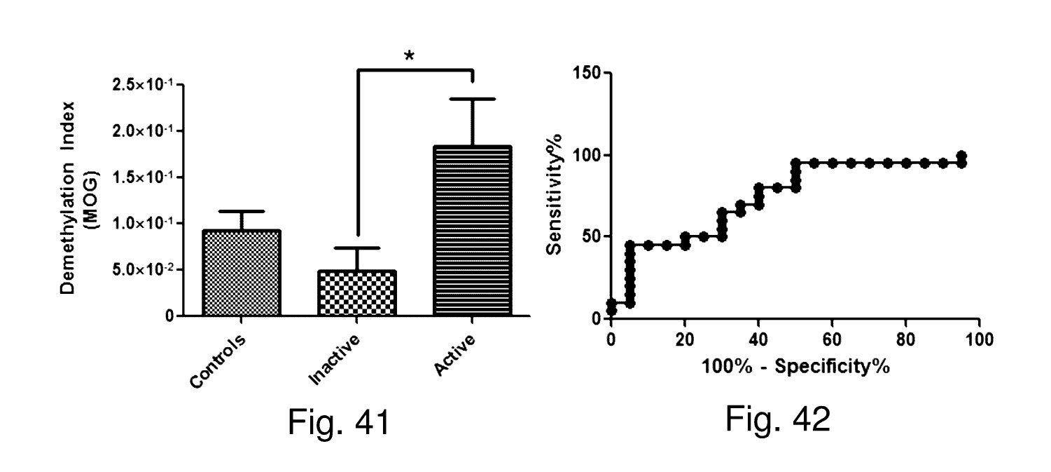

Methylation-specific primers can detect elevated levels of demethylated MOG cfDNA in patients with RRMS. FIGS. 40A and 40B provide data plots showing that methylation-specific primers can detect elevated levels of demethylated MOG cfDNA in patients with RRMS. FIG. 40A shows Age at diagnosis of relapsing-remitting multiple sclerosis for both disease active and inactive groups. FIG. 40B shows duration of disease of relapsing-remitting multiple sclerosis for both active and inactive groups. FIG. 41 shows a bargraph comparing demethylation index from methylation-specific primers were used in qPCR with bisulfite treated DNA extracted from sera from Healthy Controls, Inactive and Active RRMS patients; ANOVA p<0.029, Inactive vs. Active p<0.05. FIG. 42 shows an ROC analysis of samples showed an AUC of 0.7475 with 95% confidence interval of 0.59-0.9. These analysis reached statistical significance (p<0.007).

### Example 4

### Digital Droplet PCR

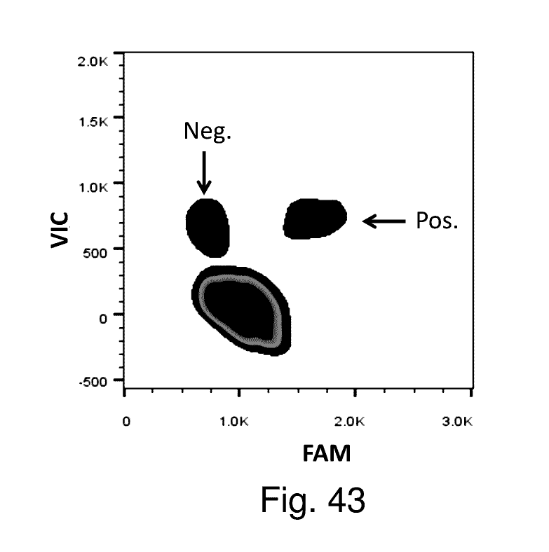

FIG. 43 shows a graph, generated using a total insulin probe and hypomethylated specific insulin probes in a ddPCR reaction using RainDance Technologies. Neg: Non-demethylated DNA originated from non-beta cells. Pos: DeMethylated DNA from beta cells. Template used are primary islet preparations from humans.

The preferred probes are ZEN™ double quenched probes. See, e.g., www.idtdna.com/pages/decoded/decoded-articles/pipet-tips/decoded/2015/04/07/qpcr-probes-selecting-the-best-reporter-dye-and-quencher; www.idtdna.com/pages/decoded/decoded-articles/competitive-edge/decoded/2013/09/16/two-quenchers-are-better-than-one!; Wilson P, Labonte M, et al. (2011) A novel fluorescence-based assay for the rapid detection and quantification of cellular deoxyribonucleoside triphosphates. Nucleic Acids Res, 1(39):e112; www.idtdna.com/pages/docs/technical-reports/fluorescence-and-fluorescence-applications.pdf?sfvrsn=US 20010129832; 20150276743; 20150184232; 20150086577; 20140248612; 20140162263; 20140080209; 20140038185; 20140005196; 20150184232; 20150086577; each of which is expressly incorporated herein by reference in its entirety.

Probes for the detection of demethylated human insulin DNA (i.e., DNA derived from pancreatic (β-cells):

Total human Insulin probe (Tins_HEX) 5′-HEX-[034]-ZEN-[035]- 3IABkFQ:

SEQ ID NO. 037
5′-CTTAAATAT
SEQ ID NO: 038
5′-ATAAAAAAAACCTC

Demethylated human Insulin probe (HYPOhINS) 5′-6-FAM-[039]-ZEN-[040]-3IABkFQ:

SEQ ID NO. 039
5′-ACCTCCCAA
SEQ ID NO 040
5′-CAAATCT

Common primers for total and demethylated human insulin probes:

SEQ ID NO. 041
hIns_meth_F: GTGCGGTTTATATTTGGTGGAAGTT
SEQ ID NO. 042
hIns_meth_R: ACAACAATAAACAATTAACTCACCCTACAA

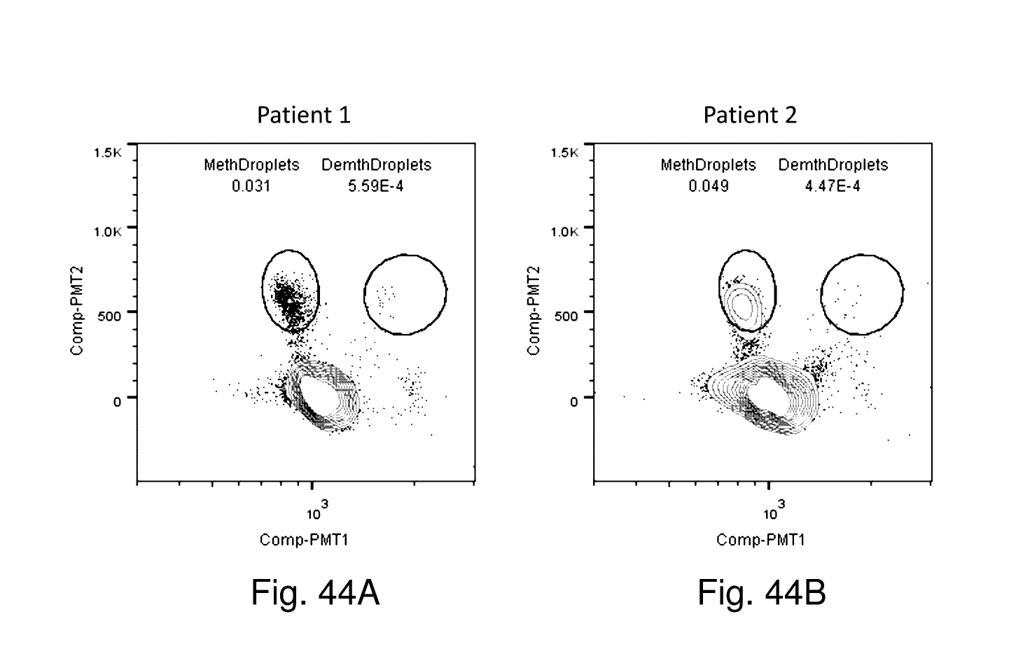

FIGS. 44A and 44B show ddPCR analysis of two patients with recent onset T1D; Both patients were positive for C-peptide indicated the presence of residual beta cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

### REFERENCES

(Each of Which is Expressly Incorporated Herein by Reference)

1. Akirav, E. M., C. M. Bergman, M. Hill, and N. H. Ruddle. 2009. Depletion of CD4(+)CD25(+) T cells exacerbates experimental autoimmune encephalomyelitis induced by mouse, but not rat, antigens. J Neurosci Res 87:3511-3519.

2. Goldenberg, M. M. 2012. Multiple sclerosis review. P T 37:175-184.

3. Bruck, W., C. Lucchinetti, and H. Lassmann. 2002. The pathology of primary progressive multiple sclerosis. Mult Scler 8:93-97.

4. Polman, C. H., S. C. Reingold, B. Banwell, M. Clanet, J. A. Cohen, M. Filippi, K. Fujihara, E. Havrdova, M. Hutchinson, L. Kappos, F. D. Lublin, X. Montalban, P. O'Connor, M. Sandberg-Wollheim, A. J. Thompson, E. Waubant, B. Weinshenker, and J. S. Wolinsky. 2011. Diagnostic criteria for multiple sclerosis: 2010 revisions to the McDonald criteria. Ann Neurol 69:292-302.

5. Rashid, W., and D. H. Miller. 2008. Recent advances in neuroimaging of multiple sclerosis. Semin Neurol 28:46-55.

6. Ceccarelli, A., R. Bakshi, and M. Neema. 2012. MRI in multiple sclerosis: a review of the current literature. Curr Opin Neurol 25:402-409.

7. Rudick, R. A., and A. E. Miller. 2012. Multiple sclerosis or multiple possibilities: the continuing problem of misdiagnosis. Neurology 78:1904-1906.

8. Boissy, A. R., and P. J. Ford. 2012. A touch of MS: therapeutic mislabeling. Neurology 78:1981-1985.

9. Solomon, A. J., E. P. Klein, and D. Bourdette. 2012. “Undiagnosing” multiple sclerosis: the challenge of misdiagnosis in MS. Neurology 78:1986-1991.

10. Wegner, C., M. M. Esiri, S. A. Chance, J. Palace, and P. M. Matthews. 2006. Neocortical neuronal, synaptic, and glial loss in multiple sclerosis. Neurology 67:960-967.

11. Prineas, J. W., and J. D. Parratt. 2012. Oligodendrocytes and the early multiple sclerosis lesion. Ann Neurol 72:18-31.

12. Akirav, E. M., J. Lebastchi, E. M. Galvan, O. Henegariu, M. Akirav, V. Ablamunits, P. M. Lizardi, and K. C. Herold. 2011. Detection of beta cell death in diabetes using differentially methylated circulating DNA. Proc Natl Acad Sci U S A 108:19018-19023.

13. Suschek, C., K. Fehsel, K. D. Kroncke, A. Sommer, and V. Kolb-Bachofen. 1994. Primary cultures of rat islet capillary endothelial cells. Constitutive and cytokine-inducible macrophagelike nitric oxide synthases are expressed and activities regulated by glucose concentration. Am J Pathol 145:685-695.

14. Akirav E, Kushner J A, Herold K C (2008) β-cell mass and type 1 diabetes: Going, going, gone? Diabetes 57:2883-2888.

15. Bluestone J A, Herold K, Eisenbarth G (2010) Genetics, pathogenesis and clinical interventions in type 1 diabetes. Nature 464:1293-1300.

16. Waldron-Lynch F, Herold KC (2009) Advances in type 1 diabetes therapeutics: Immunomodulation and β-cell salvage. Endocrinol Metab Clin North Am 38:303-317.

17. Bougneres P F, et al. (1988) Factors associated with early remission of type I diabetes in children treated with cyclosporine. N Engl J Med 318:663-670.

18. Keymeulen B, et al. (2005) Insulin needs after CD3-antibody therapy in new-onset type 1 diabetes. N Engl J Med 352:2598-2608.

19. Ludvigsson J, Heding L (1982) Abnormal proinsulin/C-peptide ratio in juvenile diabetes. Acta Diabetol Lat 19:351-358.

20. Snorgaard O, Lassen L H, Binder C (1992) Homogeneity in pattern of decline of β-cell function in IDDM: Prospective study of 204 consecutive cases followed for 7.4 yr. Diabetes Care 15:1009-1013.

21. Greenbaum C J, et al.; Type 1 Diabetes Trial Net Research Group; European C-Peptide Trial Study Group (2008) Mixed-meal tolerance test versus glucagon stimulation test for the assessment of β-cell function in therapeutic trials in type 1 diabetes. Diabetes Care 31:1966-1971.

22. Steele C, et al. (2004) Insulin secretion in type 1 diabetes. Diabetes 53:426-433.

23. Erlich H, et al.; Type 1 Diabetes Genetics Consortium (2008) HLA DR-DQ haplotypes and genotypes and type 1 diabetes risk: Analysis of the Type 1 Diabetes Genetics Consortium families. Diabetes 57:1084-1092.

24. Hagopian W A, et al. (1995) Glutamate decarboxylase, insulin, and islet cell antibodies and HLA typing to detect diabetes in a general population-based study of Swedish children. J Clin Invest 95:1505-1511.

25. Verge C F, et al. (1996) Prediction of type I diabetes in first-degree relatives using a combination of insulin, GAD, and ICA512bdc/IA-2 autoantibodies. Diabetes 45: 926-933.

26. Sherr J, Sosenko J, Skyler J S, Herold K C (2008) Prevention of type 1 diabetes: The time has come. Nat Clin Pract Endocrinol Metab 4:334-343.

27. Sosenko J M, et al.; Diabetes Prevention Trial Type 1 Study Group (2007) Increasing the accuracy of oral glucose tolerance testing and extending its application to individuals with normal glucose tolerance for the prediction of type 1 diabetes: The Diabetes Prevention Trial Type 1. Diabetes Care 30:38-42.

28. Medarova Z, Tsai S, Evgenov N, Santamaria P, Moore A (2008) In vivo imaging of a diabetogenic CD8+ T cell response during type 1 diabetes progression. Magn Reson Med 59:712-720.

29. Klose R J, Bird A P (2006) Genomic DNA methylation: The mark and its mediators. Trends Biochem Sci 31:89-97.

30. Bartke T, et al. (2010) Nucleosome-interacting proteins regulated by DNA and histone methylation. Cell 143:470-484.

31. Wang G G, Allis C D, Chi P (2007) Chromatin remodeling and cancer, part II: ATP-dependent chromatin remodeling. Trends Mol Med 13:373-380.

32. Miranda T B, Jones P A (2007) DNA methylation: The nuts and bolts of repression. J Cell Physiol 213:384-390.

33. Grady W M, Rajput A, Lutterbaugh J D, Markowitz S D (2001) Detection of aberrantly methylated hMLH1 promoter DNA in the serum of patients with microsatellite un-stable colon cancer. Cancer Res 61:900-902.

34. Wallner M, et al. (2006) Methylation of serum DNA is an independent prognostic marker in colorectal cancer. Clin Cancer Res 12:7347-7352.

35. Müller H M, et al. (2003) DNA methylation in serum of breast cancer patients: An independent prognostic marker. Cancer Res 63:7641-7645.

36. Poitout V, et al. (1995) Morphological and functional characterization of β TC-6 cells: An insulin-secreting cell line derived from transgenic mice. Diabetes 44:306-313.

37. Sherry N A, et al. (2006) Effects of autoimmunity and immune therapy on β-cell turnover in type 1 diabetes. Diabetes 55:3238-3245.

38. Jayaraman S (2011) Assessment of beta cell viability. Curr Protoc Cytom 55:6.27.1-6.27.16.

39. Kuroda A, et al. (2009) Insulin gene expression is regulated by DNA methylation. PLoS ONE 4:e6953.

40. Giddings S J, King C D, Harman K W, Flood J F, Carnaghi L R (1994) Allele-specific inactivation of insulin 1 and 2 in the mouse yolk sac indicates imprinting. Nat Genet 6:310-313.

41. Leiter E H, et al. (2007) Unexpected functional consequences of xenogeneic transgene expression in β-cells of NOD mice. Diabetes Obes Metab 9(Suppl 2):14-22.

42. Trudeau J D, et al. (2000) Neonatal β-cell apoptosis: A trigger for autoimmune diabetesβ Diabetes 49:1-7.

43. Fujimoto K, et al. (2010) Loss of Nix in Pdx1-deficient mice prevents apoptotic and necrotic β cell death and diabetes. J Clin Invest 120:4031-4039.

44. Basadonna G, Montorsi F, Kakizaki K, Merrell R C (1988) Cyclosporin A and islet function. Am J Surg 156:191-193.

45. Berney T, et al. (2006) Detection of insulin mRNA in the peripheral blood after human islet transplantion predicts deterioration of metabolic control. Am J Transplant 6:1704-1711.

46. Best C H, Haist R E, Ridout J H (1939) Diet and the insulin content of pancreas. J Physiol 97:107-119.

47. Collins T J (2007) ImageJ for microscopy. Biotechniques 43(Suppl 1):25-30.

48. Irina Botezatu, Ol'ga Serdyuk, Galina Potapova, Valery Shelepov, Raisa Alechina, Yuriy Molyaka, Vitaliy Anan'ev, Igor Bazin, August Garin, Mehti Narimanov, Vasiliy Knysh, Hovsep Melkonyan, Samuil Umansky, and Anatoly Lichtenstein (2000) “Genetic Analysis of DNA Excreted in Urine: A New Approach for Detecting Specific Genomic DNA Sequences from Cells Dying in an Organism”, Clinical Chemistry 46:8 1078-1084.

49. Umansky, S. R., et al. (1982) “In vivo DNA degradation of thymocytes of gamma-irradiated or hydrocortisone-treated rats”; Biochim. Biophys. Acta 655:9-17.

50. Lichtenstein, A. V., et al. (2001), “Circulating nucleic acids and apoptosis”; Ann NY Acad Sci, 945:239-249.

What is claimed is:

1. A method for monitoring a cell-type specific apoptosis, comprising:
extracting and purifying DNA from a body fluid of a human or animal;
treating the extracted purified DNA with bisulfite to convert demethylated cytosine to uracil while sparing the methylated cytosines;
amplifying the bisulfite-treated DNA using polymerase chain reaction;
purifying the amplified bisulfite-treated DNA;
performing a methylation sensitive probe hybridization reaction on the purified bisulfite-treated DNA using at least two different methylation specific probes which quantitatively distinguish between demethylated DNA of a gene corresponding to a selectively expressed gene product of the type of cell and methylated DNA corresponding to the same gene from other cells; and
computing a quantitative relationship between methylated DNA and demethylated DNA.

2. The method according to claim 1, wherein the polymerase chain reaction is conducting using a forward primer selected from the group consisting of:
SEQ ID NO: 001, SEQ ID NO: 004, SEQ ID NO: 007, and SEQ ID NO: 010 and a reverse primers
SEQ ID NO: 002, SEQ ID NO: 005, SEQ ID NO: 008, and SEQ ID NO: 011.

3. The method according to claim 1, wherein the at least two methylation specific probes comprise:
a) probe for detection of methylated DNA selected from the group consisting of at least one of:
SEQ ID NO: 003 and SEQ ID NO: 009. And SEQ ID NO: 004; and
b) probe for detection of demethylated DNA selected from the group consisting of at least one of:
SEQ ID NO: 005, SEQ ID NO: 006 and SEQ ID NO: 012.

4. The method according to claim 1, wherein the at least two probes are conjugated to a fluorophore.

5. The method according to claim 4, wherein the respective fluorophore is at least one of 6-carboxy fluorescein and tetrachlorofluorescein.

6. The method according to claim 4, wherein the probe comprises a quencher.

7. The method according to claim 6, wherein the quencher is tetramethylrhodamine.

8. The method according to claim 4, wherein the methylation sensitive reaction comprises quantitatively determining a release of a fluorophore from a probe bound to the purified bisulfite-treated DNA.

9. The method according to claim 1, wherein the body fluid is derived from at least one of blood, saliva, sweat, mucous, urine, and cerebrospinal fluid.

10. A method for monitoring cell death of a cell type having at least one DNA portion that has a unique DNA CpG methylation pattern as compared to other cells, which is released into body fluids upon cell death of cells of the cell type, comprising:
extracting and purifying DNA that comprises the DNA portion;
treating the extracted purified DNA with bisulfite to convert cytosine to uracil while sparing the CpG methylated cytosines;
amplifying a region of the bisulfite-treated DNA that comprises the DNA portion by polymerase chain reaction using DNA CpG methylation pattern independent primers;
determining a quantitative relationship between the DNA portion having the unique DNA CpG methylation pattern to the DNA portion lacking the unique DNA CpG methylation pattern, by employing the DNA CpG methylation pattern-specific probes;
computing a difference between the DNA portion having the unique DNA CpG methylation pattern and the DNA portion lacking the unique DNA CpG methylation pattern.

11. The method according to claim 10, wherein the DNA portion having the unique DNA CpG methylation pattern comprises at least one of an insulin and MOG gene from a pancreatic beta cell or an oligodendrocyte.

12. The method according to claim 10, wherein the polymerase chain reaction is conducting using a forward primer: SEQ ID NO: 001 or SEQ ID NO: 007 and a reverse primer SEQ ID NO: 002 or SEQ ID NO: 008.

13. The method according to claim 10, wherein the at least two methylation specific probes comprise:
a) probe for detection of methylated MOG DNA of non-oligodendrocyte origin selected from the group consisting of at least one of:
SEQ ID NO: 003 and SEQ ID NO: 009
b) probe for detection of demethylated MOG DNA of oligodendrocyte origin selected from the group consisting of at least one of:
SEQ ID NO: 005 and SEQ ID NO: 006 and SEQ ID NO: 012.

14. The method according to claim 10, wherein the at least two methylation specific probes comprise:
a) probe for detection of methylated insulin DNA of non-βcell origin selected from the group consisting of at least one of: SEQ ID NO: 003 and SEQ ID NO: 004
b) probe for detection of demethylated insulin DNA of β cell origin selected from the group consisting of at least one of: SEQ ID NO: 006.

15. The method according to claim 10, wherein the probes are conjugated to a fluorophore and a quencher.

16. The method according to claim 10, wherein the determining a quantitative relationship comprises determining a release of a fluorophore from a probe having a quencher.

17. The method according to claim 10, wherein the determining a quantitative relationship comprises determining a conformational change of the probes dependent on binding to a respective target sequence, to alter an interaction of a fluorophore and a quencher conjugated to the probes.

18. A method for monitoring cell death as a result of an autoimmune process selectively targeting a particular cell type, comprising:
extracting and purifying genomic DNA from a body fluid of a human or animal, wherein the genomic DNA comprises at least a portion of a gene that is predominantly expressed by the particular cell type and that contains a CpG methylation site;
treating the genomic DNA with bisulfite;
performing a polymerase chain reaction (PCR) with primers that flank a region of the genomic DNA that comprises the CpG methylation site;
purifying the PCR products;
melting the PCR products into single strands;
hybridizing the single-stranded PCR products with a first oligonucleotide probe capable of hybridizing with a target sequence that comprises a site corresponding to a bisulfite-converted CpG site and a second oligonucleotide probe capable of hybridizing with a target sequence that comprises a site corresponding to a bisulfite-nonconverted CpG site, and
wherein the probes each comprise a non-FRET label pair consisting of a fluorophore and a quencher, and
wherein interaction of the first oligopeptide probe or second oligopeptide probe with a respective target causes the first oligopeptide probe or second oligopeptide probe to change from a first conformation to a second conformation, thereby changing the distance between the fluorophore and quencher of said label pair, and
wherein in only one conformation do the fluorophore and quencher interact sufficiently to quench the fluorescence of the fluorophore by a predetermined amount;
quantitatively measuring fluorescent signals emitted by the first oligopeptide probe and the second oligopeptide probe; and
reporting a quantitative relationship of the fluorescent signal emitted by the first oligopeptide probe and the second oligopeptide probe, indicative of the relative amount of DNA derived from the particular cell type versus DNA derived from other cell types.

19. The method according to claim 18, wherein the primers used for the PCR comprise at least one of SEQ ID NOS: (001, 002; 004, 005; 013, 014; 019, 020) and SEQ ID NOS: (007, 008; 010, 011; 016, 017; 022, 023).

20. The method according to claim 18, wherein the first probe comprises at least one of SEQ ID NO: (003, 004, 015, 021) and the second probe comprises at least one of SEQ ID NO: (005, 006, 018, 019).

21. The method according to claim 18, wherein the fluorophore is at least one of 6-carboxy fluorescein and tetrachlorofluorescein.

22. The method according to claim 18, wherein the quencher is tetramethylrhodamine.


SEQUENCE LISTING (42 sequences)

SEQ ID NO: 1 - DNA, 25 bp, Artificial Sequence
  Forward PCR Primer for insulin gene
gtgcggtttatatttggtggaagtt

SEQ ID NO: 2 - DNA, 30 bp, Artificial Sequence
  Reverse PCR primer for insulin gene
acaacaataaacaattaactcaccctacaa

SEQ ID NO: 3 - DNA, 16 bp, Artificial Sequence
  Probe for the detection of methylated non-beta cell insulin DNA
acctcccgacgaatct

SEQ ID NO: 4 - DNA, 17 bp, Artificial Sequence
  Probe for the detection of methylated non-beta cell insulin DNA
tacctctcgtcgaatct

SEQ ID NO: 5 - DNA, 16 bp, Artificial Sequence
  Probe for the detection of demethylated beta cell insulin DNA
acctcccaacaaatct

SEQ ID NO: 6 - DNA, 17 bp, Artificial Sequence
  probe for the detection of demethylated beta cell insulin dna
tacctcccatcaaatct

SEQ ID NO: 7 - DNA, 28 bp, Artificial Sequence
  Outer Forward PCR primer for nested PCR according to Akirav et al. (2011)
ttaggggttttaaggtagggtatttggt

SEQ ID NO: 8 - DNA, 37 bp, Artificial Sequence
  Outer Reverse PCR primer for nested PCR according to Akirav et al. (2011)
accaaaaacaacaataaacaattaactcaccctacaa

SEQ ID NO: 9 - DNA, 27 bp, Artificial Sequence
  Inner Methylated Forward PCR primer for nested PCR according to Akirav et al. (2011)
gtggatgcgttttttgtttttgttggc

SEQ ID NO: 10 - DNA, 25 bp, Artificial Sequence
  Inner Methylated Reverse PCR primer for nested PCR according to Akirav et al. (2011)
caccctacaaatcctctacctcccg

SEQ ID NO: 11 - DNA, 29 bp, Artificial Sequence
  Inner Demethylated Forward PCR primer for nested PCR according to Akirav et al. (2011)
ttgtggatgtgttttttgtttttgttggt

SEQ ID NO: 12 - DNA, 25 bp, Artificial Sequence
  Inner Demethylated Reverse PCR primer for nested PCR according to Akirav et al. (2011)
caccctacaaatcctctacctccca

SEQ ID NO: 13 - DNA, 30 bp, Artificial Sequence
  Human MOG Probe set 1, hypermethylated strand forward primer
gggtagtttagagtgataggattaagatat

SEQ ID NO: 14 - DNA, 25 bp, Artificial Sequence
  Human MOG Probe set 1, hypermethylated strand reverse primer
taaaaataaaccaccctaaaaaaaa

SEQ ID NO: 15 - DNA, 25 bp, Artificial Sequence
  Human MOG Probe set 1, hypermethylated strand probe
aacgttcttcccaaaaaatatacga

SEQ ID NO: 16 - DNA, 30 bp, Artificial Sequence; identical to SEQ ID NO: 13
  Human MOG Probe set 1, hypomethylated strand forward primer

SEQ ID NO: 17 - DNA, 25 bp, Artificial Sequence
  Human MOG Probe set 1, hypomethylated strand reverse primer
taaaaataaaccaccctaaaaaaaa

SEQ ID NO: 18 - DNA, 25 bp, Artificial Sequence
  Human MOG Probe set 1, hypomethylated strand probe
aacattcttcccaaaaaatatacaa

SEQ ID NO: 19 - DNA, 30 bp, Artificial Sequence; identical to SEQ ID NO: 13
  Human MOG Probe set 2, hypermethylated strand forward primer

SEQ ID NO: 20 - DNA, 25 bp, Artificial Sequence
  Human MOG Probe set 2, hypermethylated strand reverse primer
taaaaataaaccaccctaaaaaaaa

SEQ ID NO: 21 - DNA, 30 bp, Artificial Sequence
  Human MOG Probe set 2, hypermethylated strand probe
tacgacataacaattccacttcatccccga

SEQ ID NO: 22 - DNA, 30 bp, Artificial Sequence; identical to SEQ ID NO: 13
  Human MOG Probe set 2, hypomethylated strand forward primer

SEQ ID NO: 23 - DNA, 25 bp, Artificial Sequence
  Human MOG Probe set 2, hypomethylated strand reverse primer
taaaaataaaccaccctaaaaaaaa

SEQ ID NO: 24 - DNA, 30 bp, Artificial Sequence
  Human MOG Probe set 2, hypomethylated strand probe
tacaacataacaattccacttcatccccaa

SEQ ID NO: 25 - DNA, 26 bp, Artificial Sequence
  Mouse MOG Probe set 1, hypermethylated strand forward primer
gagtgataggattagggtattttatt

SEQ ID NO: 26 - DNA, 25 bp, Artificial Sequence
  Mouse MOG Probe set 1, hypermethylated strand reverse primer
tctacatcttaatccttaccatttc

SEQ ID NO: 27 - DNA, 31 bp, Artificial Sequence
  Mouse MOG Probe set 1, hypermethylated strand probe
acccgtaacatttttcccaaaaaaaatacga

SEQ ID NO: 28 - DNA, 26 bp, Artificial Sequence
  Mouse MOG Probe set 1, hypomethylated strand forward primer
gagtgataggattagggtattttatt

SEQ ID NO: 29 - DNA, 25 bp, Artificial Sequence
  Mouse MOG Probe set 1, hypomethylated strand reverse primer
tctacatcttaatccttaccatttc

SEQ ID NO: 30 - DNA, 31 bp, Artificial Sequence
  Mouse MOG Probe set 1, hypomethylated strand probe
acccataacatttttcccaaaaaaaatacaa

SEQ ID NO: 31 - DNA, 26 bp, Artificial Sequence
  Mouse MOG Probe set 2, hypermethylated strand forward primer
gagtgataggattagggtattttatt

SEQ ID NO: 32 - DNA, 25 bp, Artificial Sequence
  Mouse MOG Probe set 2, hypermethylated strand reverse primer
tctacatcttaatccttaccatttc

SEQ ID NO: 33 - DNA, 24 bp, Artificial Sequence
  Mouse MOG Probe set 2, hypermethylated strand probe
tacgacacgacaactctacttcat

SEQ ID NO: 34 - DNA, 26 bp, Artificial Sequence
  Mouse MOG Probe set 2, hypomethylated strand forward primer
gagtgataggattagggtattttatt

SEQ ID NO: 35 - DNA, 25 bp, Artificial Sequence
  Mouse MOG Probe set 2, hypomethylated strand reverse primer
tctacatcttaatccttaccatttc

SEQ ID NO: 36 - DNA, 24 bp, Artificial Sequence
  Mouse MOG Probe set 2, hypomethylated strand probe
tacaacacaacaactctacttcat

SEQ ID NO: 37 - DNA, 9 bp, Artificial Sequence
  Total human Insulin probe (Tins_HEX) 5′-HEX-[037]-ZEN-[038]-3IABkFQ
cttaaatat

SEQ ID NO: 38 - DNA, 14 bp, Artificial Sequence
  Total human Insulin probe (Tins_HEX) 5′-HEX-[037]-ZEN-[038]- 3IABkFQ
ataaaaaaaacctc

SEQ ID NO: 39 - DNA, 9 bp, Artificial Sequence
  Demethylated human Insulin probe (HYPOhINS) 5′-6-FAM-[039]-ZEN-[040]- 3IABkFQ
acctcccaa

SEQ ID NO: 40 - DNA, 7 bp, Artificial Sequence
  Demethylated human Insulin probe (HYPOhINS) 5′-6-FAM-[039]-ZEN-[040]- 3IABkFQ
caaatct

SEQ ID NO: 41 - DNA, 25 bp, Artificial Sequence
  human Insulin forward PCR primer hIns_meth_F
gtgcggtttatatttggtggaagtt

SEQ ID NO: 42 - DNA, 30 bp, Artificial Sequence; identical to SEQ ID NO: 2
  human insulin PCR reverse primer hIns_meth_R